(19) United States
(12) Patent Application Publication
(10) Pub. No.: US 2024/0217930 A1
(43) Pub. Date: Jul. 4, 2024

(54) SARS-COV-2 MPRO INHIBITOR COMPOUNDS

(71) Applicant: HEPTARES THERAPEUTICS LIMITED, Cambridge Cambridgeshire (GB)

(72) Inventors: Miles Stuart CONGREVE, Cambridge Cambrideshire (GB); John Andrew CHRISTOPHER, Cambridge Cambrideshire (GB); Mark PICKWORTH, Cambridge Cambrideshire (GB); Chris DE GRAAF, Cambridge Cambrideshire (GB); Alicia Perez HIGUERUELO, Cambridge Cambrideshire (GB); Jonathan Stephen MASON, Cambridge Cambrideshire (GB)

(73) Assignee: HEPTARES THERAPEUTICS LIMITED, Cambridge Cambridgeshire (GB)

(21) Appl. No.: 18/553,596

(22) PCT Filed: Apr. 1, 2022
(86) PCT No.: PCT/GB2022/050836

§ 371 Date: Oct. 2, 2023

(30) Foreign Application Priority Data
Apr. 1, 2021 (GB) 2104742.8
May 17, 2021 (GB) 2107026.3

(51) Int. Cl. C07D 209/52; A61K 31/403; C07D 403/12; C07D 405/12

(52) U.S. Cl. CPC C07D 209/52; A61K 31/403; C07D 403/12; C07D 405/12

(57) ABSTRACT

The invention described herein relates to compounds of Formula (1b):

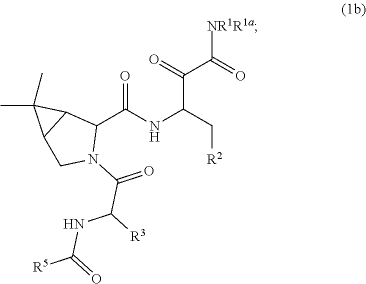

or a salt thereof, wherein R¹, R^{1a}, R², R³ and R⁵ are defined herein, and their use in the treatment of SARS-CoV-2 and related viruses and disorders associated with SARS-CoV-2.

This application relates to novel compounds and their use as SARS-CoV-2 Main Protease (Mpro) inhibitors. Compounds described herein may be useful in the treatment of SARS-CoV-2 and related viruses and disorders associated with SARS-CoV-2: Mpro. The application is also directed to pharmaceutical compositions comprising these compounds and the manufacture and use of these compounds and compositions in the treatment of SARS-CoV-2 and related viruses and disorders associated with SARS-CoV-2: Mpro. The compounds and compositions may be useful in preventing death or complications arising due to chronic underlying conditions or comorbidities in patients infected with SARS-CoV-2 and related viruses.

### BACKGROUND OF THE INVENTION

Coronaviruses have long existed in nature and have made zoonotic transmission to humans, generally causing mild respiratory illnesses such as the common cold upon infection. However, in the last two decades outbreaks of novel human coronavirus infections that cause severe respiratory illness have presented a major global health concern. This includes the severe acute respiratory syndrome coronavirus (SARS-CoV) outbreak in 2002-2004, the Middle East respiratory syndrome coronavirus (MERS-CoV) outbreak in 2012-2015 and severe acute respiratory syndrome coronavirus 2 (SARS-CoV-2), the most recently emerged strain of coronavirus, that was identified in Wuhan, China, in 2019 and is the aetiological agent responsible for the 2019-2020 viral pneumonia outbreak of coronavirus disease 2019 (COVID-19). Despite the tragic and widespread effects of these sudden occurrences and the periodic emergence of novel human coronaviruses increasing the potential for future outbreaks, we do not yet have validated antiviral treatments targeting coronavirus infections.

SARS-CoV-2 packages a large RNA genome of ˜30 kb, two-thirds of which encodes for the two polyproteins pp1a and pp1 b (Hegyi et al. Journal of General Virology 83 (3): 595-99). These polyproteins are processed into 16 non-structural proteins (nsps) that are liberated from the long polypeptide chains by two viral cysteine proteases, the papain-like protease (nsp3) and the 3C-like protease (nsp5). The latter species, also referred to as the main protease (Mpro), cleaves the viral polyproteins at eleven sites to generate twelve non-structural proteins (nsp5-16). Included in these nsps are those involved in the replication and transcription machinery such as the RNA-dependent RNA polymerase (nsp12) and helicase (nsp13). The essential role Mpro plays in viral replication has been demonstrated in mutagenesis experiments (Kim et al. Virology 208 (1): 1-8; Stobart et al. Journal of Virology 86 (9): 4801-10), which makes it an attractive target for the design of inhibitors to treat coronavirus infection. Furthermore, there are no human proteases with similar cleavage specificity and therefore selective inhibitors of Mpro are highly likely to be non-toxic (Anand et al. 2003. Science 300 (5626): 1763-67).

The use of protease inhibitors for the treatment of viral diseases is well precedented (Bacon et al. The New England Journal of Medicine 364 (13): 1207-17) and the similarity of the SARS-CoV-2 Mpro active site to other viral proteases has driven efforts to identify clinically approved drugs that could be repurposed for the treatment of COVID-19 (Riva et al. Nature, 586: 113-119). Screening of a selection of 18 viral protease inhibitors designed for the treatment of human immunodeficiency virus (HIV) and Hepatitis C virus (HCV) identified the anti-HCV drug boceprevir and the pre-clinical inhibitor against feline infectious peritonitis virus (FIPV) GC376 as inhibitors of SARS-CoV-2 Mpro (Fu et al. Nature Communications 11 (1): 4417). While GC376 showed a more potent inhibition efficacy of recombinant protease activity (IC₅₀=0.15 μM) than boceprevir (IC₅₀=8 μM), GC376 has shown side effects in trials performed in cats raising potential safety concerns (Pedersen et al. Journal of Feline Medicine and Surgery 20 (4): 378-92). Boceprevir was also identified as an inhibitor of SARS-CoV-2 Mpro alongside telaprevir in a different study, albeit both drugs inhibited SARS-CoV-2 Mpro with IC₅₀ values of >1 μM (Anson et al. 2020. doi:10.21203/rs.3.rs-26344/v1). In addition to SARS-CoV-2 Mpro, the inhibitory efficacy of boceprevir and telaprevir was also assessed at Mpro proteases from eight other coronaviruses including SARS, MERS, HKU1, HKU4, HKU5, NL63, FIPV and IBV. Within this selection boceprevir was able to inhibit all coronavirus proteases tested except NL63 and a similarly broad spectrum of activity was shown for telaprevir with inhibitory activity shown at SARS, HKU4, HKU5, NL63 and IBV. While the antiviral activity of these drugs at SARS-CoV-2 Mpro is not sufficient for clinical development, their ability to inhibit a broad range of proteases highlights the potential for the design of broad-spectrum antiviral drugs able to treat not only SARS-CoV-2 infection but also other human coronaviruses and potentially novel coronaviruses that could emerge in the future.

The sequence similarly between SARS-CoV and SARS-CoV-2 Mpro active sites was also exploited in the identification of the SARS-CoV-2 Mpro inhibitor PF-07304814, a phosphate prodrug of PF-00835231 which was originally designed for the treatment of SARS-CoV (Boras et al. BioRxiv, 2020.09.12.293498). PF-00835231 inhibited SARS-CoV-2 Mpro with a Kᵢ of 0.27 nM and displayed broad inhibitory activity against ten further coronavirus strains with Kᵢ values of 0.03-4 nM. This translated into ˜1 μM activity in cell-based live virus assays. The activity of PF-00835231 in combination with remdesivir, a nucleoside RNA-dependent RNA polymerase inhibitor, was also evaluated as antiviral agents that target different aspects of the viral replication process can yield synergistic effects in combination. Indeed, PF-00835231 and remdesivir displayed either synergistic or additive effects in a cell-based antiviral assay, which suggests that the combination of Mpro inhibitors with antivirals with other modes of actions could show clinical benefit.

In 2020 the crystal structure of SARS-CoV-2 Mpro in complex with N3 (a Michael acceptor inhibitor) was published (Jin et al. Nature 582 (7811): 289-93), thereby enabling virtual screening and structure-based drug design (SBDD) for inhibitors of SARS-CoV-2 Mpro. Such SBDD efforts included the design of peptidomimetic α-ketoamides as broad-spectrum inhibitors of coronaviruses and enteroviruses with the two most promising inhibitors showing 0.71-12.27 μM IC₅₀ values in recombinant inhibition assays for proteases from enteroviruses EV-A71 and CVB3 as well as coronaviruses SARS-CoV and NL63 (Zhang et al. 2020. Journal of Medicinal Chemistry 63 (9): 4562-4578). The activity observed in the recombinant protease assays broadly matched antiviral activity in cell-based live virus assays with IC₅₀ values within 10-fold in both systems, suggesting that good activity in the protease inhibition assay is a good indicator of antiviral activity.

Currently, there are no targeted therapeutic agents for the treatment of COVID-19, and effective treatment options remain very limited. Despite much ongoing research activity and numerous clinical trials in progress, only remdesivir and favipiravir have been approved in selected countries for limited use to treat SARS-CoV-2 infection but show only modest effects (Zhou et al. ACS Pharmacology & Translational Science 3 (5): 813-834). There exists a need for targeted therapeutic agents for the treatment of SARS-CoV-2 infection and for the reasons outlined above SARS-CoV-2 Mpro represents an attractive drug target for SARS-CoV-2. The compounds disclosed herein are shown to be inhibitors of SARS-CoV-2 Mpro and therefore represent potential candidates for the treatment of coronavirus infection and associated disorders including but not limited to COVID-19.

### The Invention

The present invention provides compounds having activity as SARS-CoV-2: Mpro inhibitors.

The invention provides a compound of Formula (1 b):

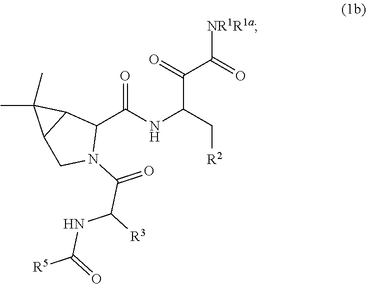

or a salt thereof, wherein:
R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Compounds of the present invention may be used as SARS-CoV-2: Mpro inhibitors. Compounds of the present invention may be used in the treatment of SARS-CoV-2 and related viruses or a disease or disorder associated with SARS-CoV-2. Compounds of the present invention may be useful in preventing death or complications arising due to chronic underlying conditions or comorbidities in patients infected with SARS-CoV-2 and related viruses. Such chronic underlying conditions or comorbidities may include for example hypertension, obesity, chronic lung conditions (TB, asthma and cystic fibrosis), diabetes and cardiovascular conditions (coronary heart disease, congenital heart disease and heart failure). Compounds of the present invention may be used in the manufacture of medicaments. The compounds or medicaments may be for use in treating, preventing, ameliorating, controlling or reducing the risk of SARS-CoV-2 and related viruses and diseases or disorders in which SARS-CoV-2: Mpro is involved. The compounds or medicaments may be for use in treating, preventing, ameliorating, controlling or reducing the risk of chronic underlying conditions or comorbidities in patients infected with SARS-CoV-2 and related viruses.

Compounds of the present invention may be for use as a single agent or in combination with one or more additional pharmaceutical agents. Compounds of the present invention may be useful in the treatment of SARS-CoV-2 and related viruses or conditions or symptoms related thereto.

### DETAILED DESCRIPTION OF THE INVENTION

The invention relates to novel compounds. The invention also relates to the use of novel compounds as inhibitors of SARS-CoV-2: Mpro. The invention further relates to the use of novel compounds in the manufacture of medicaments for use as SARS-CoV-2: Mpro inhibitors. The invention further relates to compounds, compositions and medicaments that may be useful in the treatment of SARS-CoV-2 and related viruses or conditions or symptoms related thereto.

The invention provides a compound of Formula (1b):

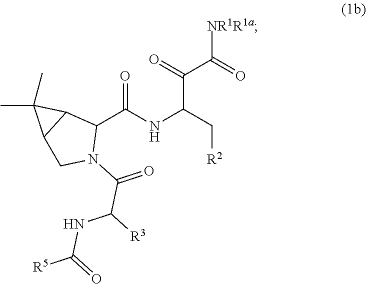

or a salt thereof, wherein:
R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Also provided is a compound of Formula (1a):

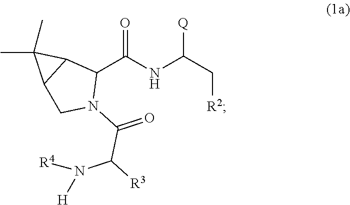

or a salt thereof, wherein;
Q is CN or a group of formula:

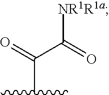

R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl, or R² is a saturated ring containing an oxygen heteroatom optionally substituted with one or more substituents chosen from fluorine, methyl or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁴ is H or CO—R⁵ wherein R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Also provided is a compound of Formula (1):

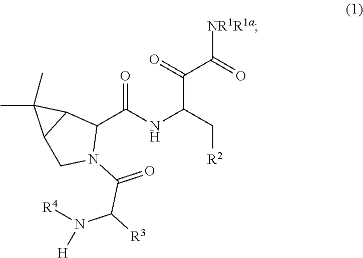

or a salt thereof, wherein;
R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl, or R² is a saturated ring containing an oxygen heteroatom optionally substituted with one or more substituents chosen from fluorine, methyl or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁴ is H or CO—R⁵ wherein R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Provided is a compound of Formula (2):

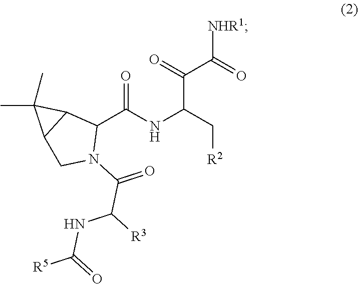

or a salt thereof, wherein;
R¹ is H, a C₁₋₆ saturated hydrocarbon group optionally substituted with 1 to 6 fluorine or chlorine atoms or a benzyl group optionally substituted with 1 to 6 fluorine or chlorine atoms;
R² is a C₃₋₅ saturated hydrocarbon group containing a cycloalkyl group optionally substituted with one or more substituents chosen from fluorine or hydroxyl, or R² is a saturated ring containing an oxygen heteroatom optionally substituted with one or more substituents chosen from fluorine, methyl or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Provided is a compound of Formula (2):

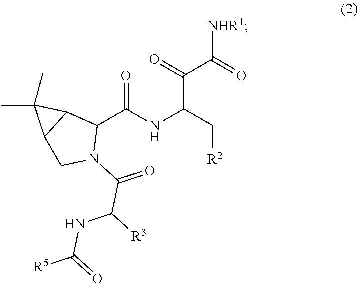

or a salt thereof, wherein;
R¹ is H, a C₁₋₆ saturated hydrocarbon group optionally substituted with 1 to 6 fluorine or chlorine atoms or a benzyl group optionally substituted with 1 to 6 fluorine or chlorine atoms;
R² is a C₃₋₅ saturated hydrocarbon group containing a cycloalkyl group;
R³ is a saturated group containing 3-5 carbon atoms and optionally containing a cycloalkyl group or optionally containing a saturated ring containing an oxygen heteroatom; and
R⁴ is a C₂₋₈ hydrocarbon group, optionally substituted with 1 to 6 fluorine or chlorine atoms and optionally containing one or more rings or a double bond.

Provided is a compound of Formula (3):

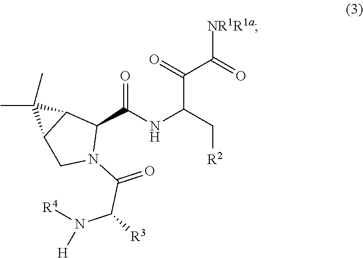

or a salt thereof, wherein;
R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl, or R² is a saturated ring containing an oxygen heteroatom optionally substituted with one or more substituents chosen from fluorine, methyl or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁴ is H or CO—R⁵ wherein R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Provided is a compound of Formula (2b):

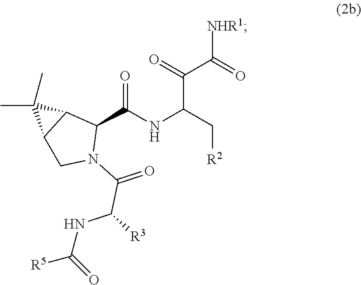

or a salt thereof, wherein;
R¹ is H, a C₁₋₆ saturated hydrocarbon group optionally substituted with 1 to 6 fluorine or chlorine atoms or a benzyl group optionally substituted with 1 to 6 fluorine or chlorine atoms;
R² is a C₃₋₅ saturated hydrocarbon group containing a cycloalkyl group optionally substituted with one or more substituents chosen from fluorine or hydroxyl, or R² is a saturated ring containing an oxygen heteroatom optionally substituted with one or more substituents chosen from fluorine, methyl or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁵ is a C₂₋₈ hydrocarbon group, optionally substituted with 1 to 6 fluorine or chlorine atoms and optionally containing one or more rings or a double bond optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

Provided is a compound of Formula (2b):

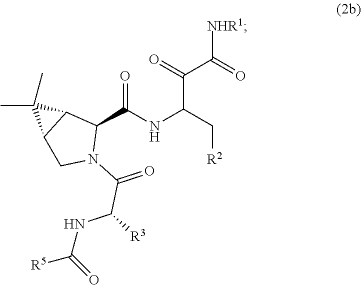

or a salt thereof, wherein;
R¹ is H, a C₁₋₆ saturated hydrocarbon group optionally substituted with 1 to 6 fluorine or chlorine atoms or a benzyl group optionally substituted with 1 to 6 fluorine or chlorine atoms;
R² is a C₃₋₅ saturated hydrocarbon group containing a cycloalkyl group;
R³ is a saturated group containing 3-5 carbon atoms and optionally containing a cycloalkyl group or optionally containing a saturated ring containing an oxygen heteroatom; and
R⁵ is a C₂₋₈ hydrocarbon group, optionally substituted with 1 to 6 fluorine or chlorine atoms and optionally containing one or more rings or a double bond.

In the compounds herein, Q can be CN. Q can be a group of formula:

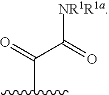

Q can be selected from the group consisting of:

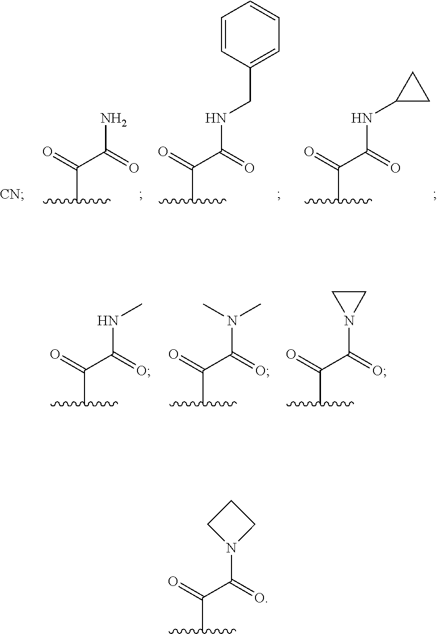

In the compounds herein, R^{1a} can be H. R^{1a} can be a C₁₋₆ saturated hydrocarbon group optionally substituted with 1 to 6 fluorine or chlorine atoms. R^{1a} can be methyl. R^{1a} can be cyclopropyl. R^{1a} can be a benzyl group optionally substituted with 1 to 6 fluorine or chlorine atoms. R^{1a} can be selected from the group consisting of —CH₂-cyclopropyl, —CH₂-chlorophenyl, —CH₂-phenyl, methylcyclopropyl, 1,1-dimethylcyclopropyl, 1,2-dimethylcyclopropyl, difluorocyclopropyl.

In the compounds herein, R¹ can be H. R¹ can be a C₁₋₆ saturated hydrocarbon group optionally substituted with 1 to 6 fluorine or chlorine atoms. R¹ can be methyl. R¹ can be cyclopropyl. R¹ can be a benzyl group optionally substituted with 1 to 6 fluorine or chlorine atoms. R¹ can be selected from the group consisting of —CH₂-cyclopropyl, —CH₂-chlorophenyl, —CH₂-phenyl, methylcyclopropyl, 1,1-dimethylcyclopropyl, 1,2-dimethylcyclopropyl, difluorocyclopropyl.

R¹ can be selected from the group consisting of:

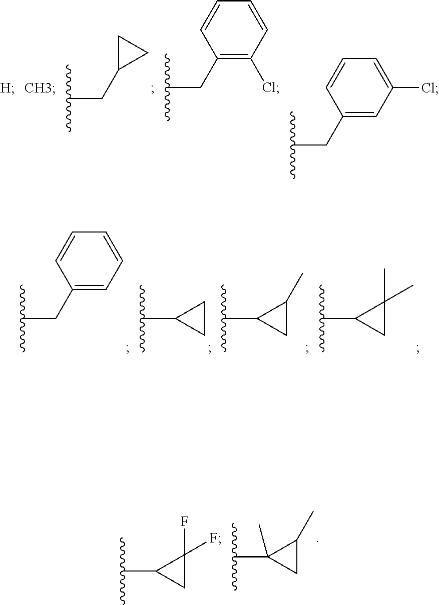

R¹ can be H, CH₃, benzyl, cyclopropyl or

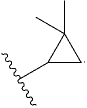 [SMILES: *C1CC1(C)C]

R^{1a} can be selected from the group consisting of H and methyl. R^{1a} can be H. R¹ and R^{1a} can both be H. R¹ and R^{1a} can both be —CH₃.

R¹ and R^{1a} may be linked together to form a saturated ring optionally containing an additional heteroatom. The ring may contain 3 to 6 atoms. The heteroatom may be O or N. The heteroatom may be O. The ring may be an alkyl chain (CH₂)n where n is 2 to 5. n may be 2, 3, 4 or 5. R¹ and R^{1a} may be joined to form a 3 to 6-membered ring. R¹ and R^{1a} may be joined to form an aziridine ring, an azetidine ring, a pyrollidine ring, a piperidine ring or a morpholine ring. R¹ and R^{1a} may be joined to form an azetidine ring. R¹ and R^{1a} can be linked together to form an azetidine or aziridine ring.

In the compounds herein, R² can be a C₃₋₅ saturated hydrocarbon group containing a cycloalkyl group optionally substituted with one or more substituents chosen from fluorine or hydroxyl, or R² can be a saturated ring containing an oxygen heteroatom optionally substituted with one or more substituents chosen from fluorine, methyl or hydroxyl. R² can be a C₃₋₅ saturated hydrocarbon group containing a cycloalkyl group optionally substituted with one or more substituents chosen from fluorine or hydroxyl. R² can be a C₃₋₅ saturated hydrocarbon group containing a cycloalkyl group. R² can be a saturated ring containing an oxygen heteroatom. The ring can contain 3 to 6 atoms, one of which is O. The ring can be optionally substituted with one or more substituents chosen from fluorine, methyl or a hydroxyl group. R² can be selected from the group consisting of cyclobutyl, cyclopropyl, methylcyclopropyl.

R² can be selected from the group consisting of:

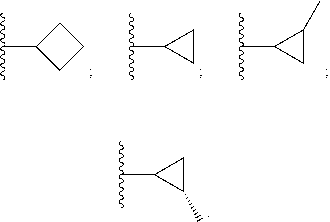

R² can be selected from the group consisting of:

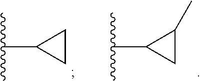

R² can be:

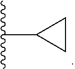 [SMILES: *C1CC1]

In the compounds herein, R³ can be a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ can be CH₂aryl, CH(CH₃)aryl or C(CH₃)₂aryl. R³ can be a saturated group containing 3-5 carbon atoms and optionally containing a cycloalkyl group or optionally containing a saturated ring containing an oxygen heteroatom and optionally substituted with one or more substituents chosen from fluorine, or hydroxyl. R³ can be a saturated group containing 3-5 carbon atoms and containing a cycloalkyl group. R³ can be a saturated group containing a saturated ring containing an oxygen heteroatom. The ring can contain 3 to 6 atoms, one of which is O. The ring may contain one or more substituents chosen from fluorine, methyl or a hydroxyl group. R³ can be CH₂aryl, CH(CH₃)aryl or C(CH₃)₂aryl. The aryl group may be phenyl. R³ can be selected from the group consisting of —CH₂-cyclopropyl, —CH(CH₃)₂, —C(CH₃)₃, cyclopropyl, oxolane, oxetane, —CH(CH₃)CH₂CH₃, cyclobutyl, C(CH₃)₂Ph, CH₂Ph.

R³ can be selected from the group consisting of:

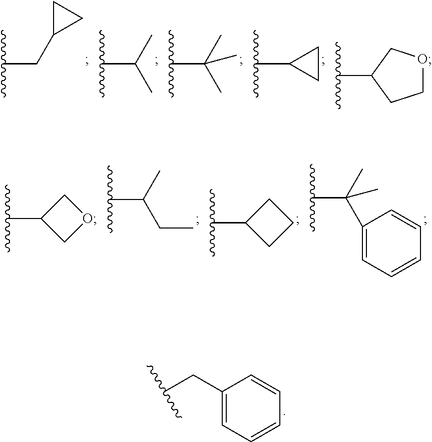

In the compounds herein, R⁴ can be H or CO—R⁵ wherein R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

In the compounds herein, R⁵ can be a C₂₋₈ hydrocarbon group, optionally substituted with 1 to 6 fluorine or chlorine atoms and optionally containing one or more rings or a double bond optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

R⁵ can be a C₂₋₈ hydrocarbon group, optionally containing one or more rings or a double bond. R⁵ can be selected from the group consisting of cyclopropyl, cyclobutyl, cyclopentyl, ethyl, —CH(CH₃)₂, —CH₂(CH₃)₂—CH(CH₃)CH₂CH₃, —CH₂CF₃, CH(CF₃)₂, methylcyclopropyl, vinyl (—CH═CH₂), bicyclo(1,1,1)pentane and cubane.

R⁵ can be selected from the group consisting of:

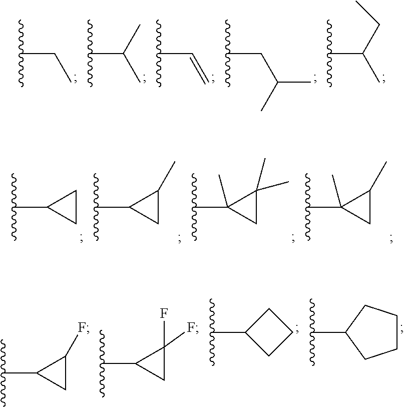

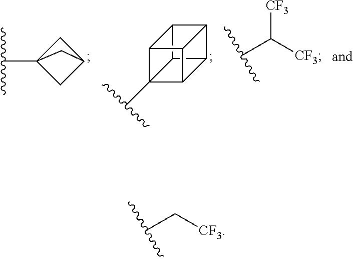

R⁵ can be selected from the group consisting of:

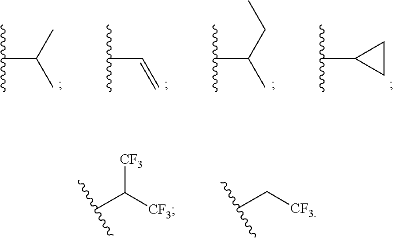

Provided are compounds of Formula (1):

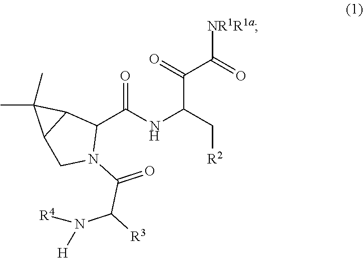

or a salt thereof, wherein;
R^{1a} is H or methyl;
R¹ is H, methyl, benzyl or cyclopropyl;
or R¹ and R^{1a} are linked together to form a saturated ring of 3 to 6 atoms;
R² is selected from the group consisting of:

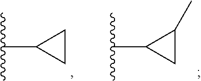

R³ is selected from the group consisting of:

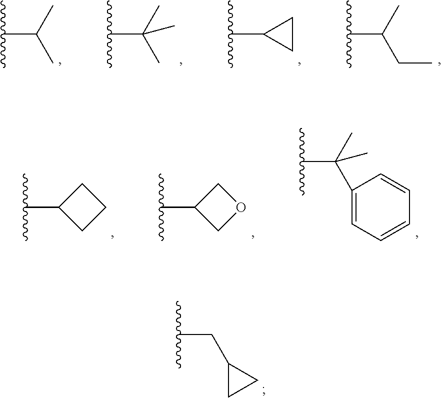

and
R⁴ is CO—R⁵ wherein R⁵ is selected from the group consisting of:

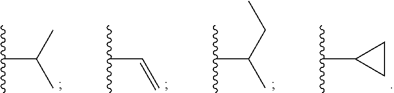

The compounds can be compounds of Formula (1a), (1b), (1) or (1i):

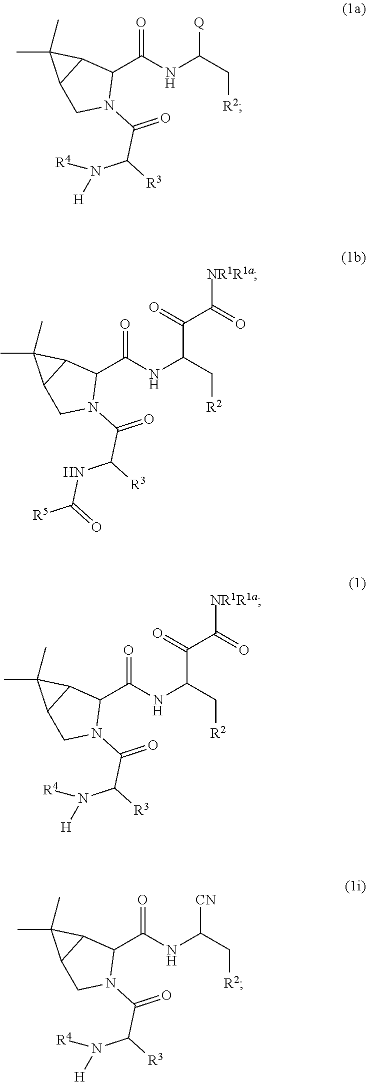

or a salt thereof, wherein Q, R¹, R^{1a}, R², R³, R⁴ and R⁵ are as defined herein.

The compounds can be compounds of Formula (2), (2a), (2i), (2b), (2ba) or (2bi):

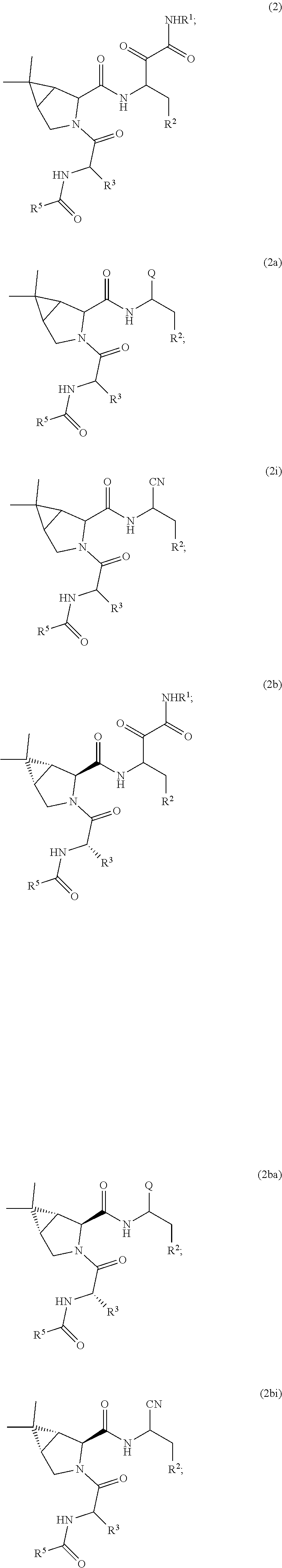

or a salt thereof, wherein Q, R¹, R², R³ and R⁵ are as defined herein.

The compounds can be compounds of Formula (3), (3a), (3b) or (3i):

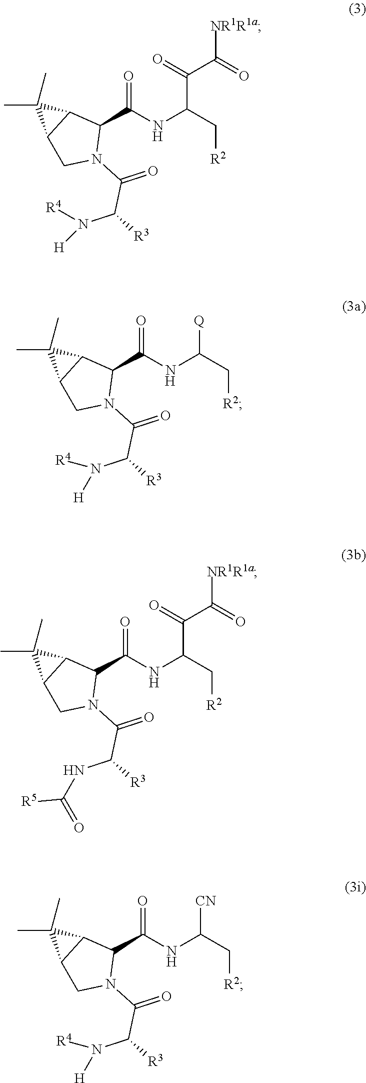

or a salt thereof, wherein Q, R¹, R^{1a}, R², R³, R⁴ and R⁵ are as defined herein.

The compound can be selected from any one of Examples 1 to 35 as shown in Table 1 or an isomer or salt thereof.

The compound can be selected from the group consisting of:
(1 R,2S,5S)—N-(4-Amino-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-[3-Amino-1-(cyclopropylmethyl)-2,3-dioxo-propyl]-3-[(2S)-2-(cyclopropanecarbonylamino)-3-methylbutanoyl]-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-alloisoleucyl)-6,6-dimethyl-3-azabicyclo[3.1.0]hexane-2-carboxamide;
(1 R,2S,5S)-3-(Acryloyl-L-valyl)-N-(4-amino-1-cyclopropyl-3,4-dioxobutan-2-yl)-6,6-dimethyl-3-azabicyclo[3.1.0]hexane-2-carboxamide;
(1 R,2S,5S)—N-(4-Amino-1-((1 R,2S)-2-methylcyclopropyl)-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3-cyclopropyl-2-isobutyramidopropanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-6,6-dimethyl-3-(((S)-2-methylbutanoyl)-L-valyl)-3-azabicyclo[3.1.0]hexane-2-carboxamide;
(1 R,2S,5S)—N-(4-Amino-1-cyclopropyl-3,4-dioxobutan-2-yl)-3-((S)-2-(cyclopropanecarboxamido)-2-cyclopropylacetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-cyclopropyl-2-isobutyramidoacetyl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-(isobutyryl-L-valyl)-6,6-dimethyl-3-azabicyclo[3.1.0]hexane-2-carboxamide;
(1 R,2S,5S)—N-(1-Cyclopropyl-4-(cyclopropylamino)-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(2-isobutyramido-2-(oxetan-3-yl)acetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-(cyclopropanecarboxamido)-3,3-dimethylbutanoyl)-6,6-dimethyl-3-azabicyclo[3.1.0]hexane-2-carboxamide;
(1 R,2S,5S)-3-((S)-2-Acrylamido-3,3-dimethylbutanoyl)-N-(4-amino-1-cyclopropyl-3,4-dioxobutan-2-yl)-6,6-dimethyl-3-azabicyclo[3.1.0]hexane-2-carboxamide;
(1 R,2S,5S)-3-((S)-2-Acrylamido-3,3-dimethylbutanoyl)-N-(4-(benzylamino)-1-cyclopropyl-3,4-dioxobutan-2-yl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-((S)-2-(cyclopropanecarboxamido)-3,3-dimethylbutanoyl)-6,6-dimethyl-3-azabicyclo[3.1.0]hexane-2-carboxamide;
(1 R,2S,5S)-3-((S)-2-(Cyclopropanecarboxamido)-3,3-dimethylbutanoyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)-3-((S)-2-Acrylamido-3,3-dimethylbutanoyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)—N-(1-Cyclopropyl-4-(cyclopropylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(1-Cyclopropyl-4-(methylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(1-Cyclopropyl-4-(dimethylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-(Aziridin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-cyclobutyl-2-isobutyramidoacetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-isobutyramido-3-methyl-3-phenylbutanoyl)-6,6-dimethyl-3-azabicyclo[3.1.0]hexane-2-carboxamide;
(1 R,2S,5S)—N-(1-Cyano-2-cyclopropylethyl)-3-((S)-2-isobutyramido-3,3-dimethylbutanoyl)-6,6-dimethyl-3-azabicyclo[3.1.0]hexane-2-carboxamide;
(1 R,2S,5S)—N-(4-(Azetidin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)-3-((Cyclopropanecarbonyl)-L-valyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(2-isobutyramido-2-(tetrahydrofuran-3-yl)acetyl)-6,6-dimethyl-3-azabicyclo[3.1.0]hexane-2-carboxamide;
(1 R,2S,5S)—N-(1-Cyclopropyl-4-(((S)-2,2-dimethylcyclopropyl)amino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-phenylalanyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3,3-dimethyl-2-(3,3,3-trifluoro-2-(trifluoromethyl)propanamido)butanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide;
(1 R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide;
   or a salt thereof.

Further embodiments include the use of a compound of the invention or a salt thereof or a pharmaceutical composition comprising a compound of the invention as a SARS-CoV-2: Mpro inhibitor. Compounds of the present invention may be used as SARS-CoV-2: Mpro inhibitors. Compounds of the present invention may be used in the treatment of SARS-CoV-2 or a disease or disorder associated with SARS-CoV-2. Compounds of the present invention may be useful in preventing death or complications arising due to chronic underlying conditions or comorbidities in patients infected with SARS-CoV-2. Such chronic underlying conditions or comorbidities may include for example hypertension, obesity, chronic lung conditions (TB, asthma and cystic fibrosis), diabetes and cardiovascular conditions (coronary heart disease, congenital heart disease and heart failure). Compounds of the present invention may be used in the manufacture of medicaments. The compounds or medicaments may be for use in treating, preventing, ameliorating, controlling or reducing the risk of SARS-CoV-2 and diseases or disorders in which SARS-CoV-2: Mpro is involved. The compounds or medicaments may be for use in treating, preventing, ameliorating, controlling or reducing the risk of chronic underlying conditions or comorbidities in patients infected with SARS-CoV-2.

Compounds of the present invention may be for use as a single agent or in combination with one or more additional pharmaceutical agents. Compounds of the present invention may be useful in the treatment of SARS-CoV-2 or conditions or symptoms related thereto.

As provided herein, compounds or salts thereof described herein and compositions described herein may be administered with an agent to treat any of the diseases and disorders disclosed herein.

The compounds of the invention may be presented in the form of a prodrug. By “prodrug” is meant for example any compound that is converted in vivo into a biologically active compound of the invention. For example, some prodrugs are esters or phosphate esters of the active compound (e.g., a physiologically acceptable metabolically labile ester). During metabolism, the ester group (—C(═O)OR) or phosphate ester group (P(═O)(OH)₂—OR) is cleaved to yield the active drug. Such esters may be formed by esterification, for example, of an hydroxyl group present in the parent compound with, where appropriate, prior protection of any other reactive groups present in the parent compound, followed by deprotection if required. Other functionality present in the active compound, for example an amide group or amino group, can be used to form a prodrug. Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Accordingly, provided is a prodrug of a compound as defined herein wherein the compound contains a functional group which is convertible under physiological conditions to form a hydroxyl group, amide group or amino group.

### Definitions

In this application, the following definitions apply, unless indicated otherwise.

The term “SARS-CoV-2: Mpro inhibitor” as used herein refers to any compound which binds to and modulates the function of SARS-CoV-2: Mpro.

The term “treatment”, in relation to the uses of any of the compounds described herein, including those of Formula (1 b) is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

Terms such as “benzyl” “bicyclic”, “hydrocarbon”, “heterocyclic”, “carbocyclic”, “alkyl”, “aryl”, “amino” “heteroaryl”, “cycloalkyl” and “halo” are all used in their conventional sense (e.g. as defined in the IUPAC Gold Book), unless indicated otherwise. “optionally substituted” as applied to any group means that the said group may if desired be substituted with one or more substituents, which may be the same or different.

The term “saturated hydrocarbon group” as in “C₁₋₄ saturated hydrocarbon group” refers to a hydrocarbon group containing no carbon-carbon double bonds or triple bonds. The saturated hydrocarbon group can therefore be an alkyl group, a cycloalkyl group, a cycloalkylalkyl group, an alkylcycloalkyl group or an alkylcycloalkylalkyl group. Examples of C₁₋₄ saturated hydrocarbon groups include C₁₋₄ alkyl groups, cyclopropyl, cyclobutyl and cyclopropylmethyl. The term “cycloalkyl” as used herein, where the specified number of carbon atoms permits, includes both monocyclic cycloalkyl groups such as cyclopropyl, cyclobutyl, cyclopentyl, cyclohexyl and cycloheptyl, and bicyclic and tricyclic groups. Bicyclic cycloalkyl groups include bridged ring systems such as bicycloheptane, bicyclooctane and adamantane.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g.(+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³ and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in some embodiments of the invention, there is provided a pharmaceutical composition comprising at least one compound of the invention together with at least one pharmaceutically acceptable excipient.

The pharmaceutically acceptable excipient(s) can be selected from, for example, carriers (e.g. a solid, liquid or semi-solid carrier), adjuvants, diluents (e.g. solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the invention can be formulated in accordance with known techniques, see for example, Remington's Pharmaceutical Sciences, Mack Publishing Company, Easton, PA, USA. The pharmaceutical compositions can be in any form suitable for oral, parenteral, intravenous, intramuscular, intrathecal, subcutaneous, topical, intranasal, intrabronchial, sublingual, buccal, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The composition may be a tablet composition or a capsule composition. Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments.

Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

The composition may be a parenteral composition. Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds of the invention will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

The compounds may be administered alongside other agents, for example other agents used in treating subjects with SARS-CoV-2. The compounds may be co-administered with HIV drugs which are known to block cypP450 mediated metabolism, such as ritonavir or a combination of lopinavir/ritonavir.

### EXAMPLES

The invention will now be illustrated, but not limited, by reference to the following examples shown in Table 1.

TABLE 1

| | |
|---|---|
| 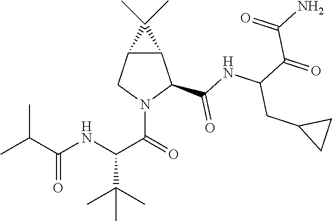 [SMILES: CC(C)C(=O)N[C@H](C(=O)N1C[C@H]2[C@@H]([C@H]1C(=O)NC(CC1CC1)C(=O)C(N)=O)C2(C)C)C(C)(C)C] | Example 1 |
| 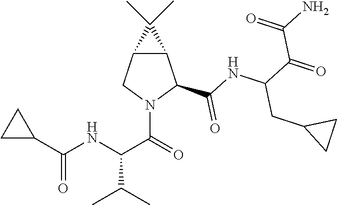 [SMILES: CC(C)[C@H](NC(=O)C1CC1)C(=O)N1C[C@H]2[C@@H]([C@H]1C(=O)NC(CC1CC1)C(=O)C(N)=O)C2(C)C] | Example 2 |
| 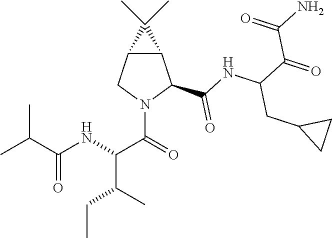 | Example 3 |
| 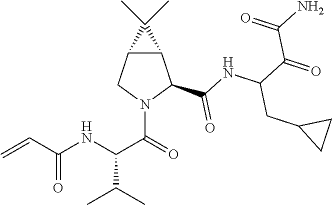 [SMILES: C=CC(=O)N[C@H](C(=O)N1C[C@H]2[C@@H]([C@H]1C(=O)NC(CC1CC1)C(=O)C(N)=O)C2(C)C)C(C)C] | Example 4 |
| 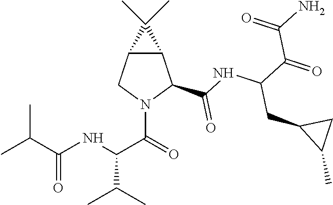 [SMILES: CC(C)C(=O)N[C@H](C(=O)N1C[C@H]2[C@@H]([C@H]1C(=O)NC(C[C@H]1C[C@@H]1C)C(=O)C(N)=O)C2(C)C)C(C)C] | Example 5 |
| 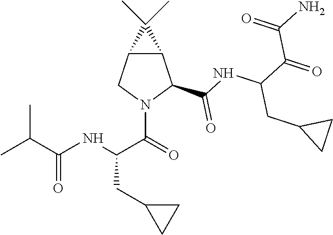 [SMILES: CC(C)C(=O)N[C@@H](CC1CC1)C(=O)N1C[C@H]2[C@@H]([C@H]1C(=O)NC(CC1CC1)C(=O)C(N)=O)C2(C)C] | Example 6 |
| 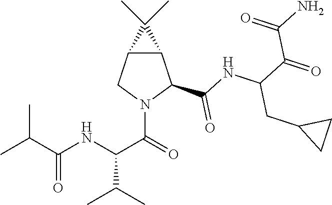 [SMILES: CC(C)C(=O)N[C@H](C(=O)N1C[C@H]2[C@@H]([C@H]1C(=O)NC(CC1CC1)C(=O)C(N)=O)C2(C)C)C(C)C] | Example 7 |
| 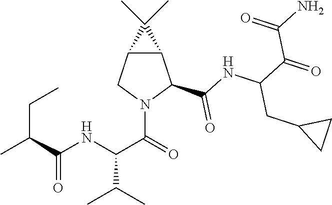 [SMILES: CC[C@H](C)C(=O)N[C@H](C(=O)N1C[C@H]2[C@@H]([C@H]1C(=O)NC(CC1CC1)C(=O)C(N)=O)C2(C)C)C(C)C] | Example 8 |
| 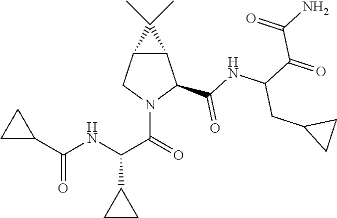 [SMILES: CC1(C)[C@@H]2[C@@H](C(=O)NC(CC3CC3)C(=O)C(N)=O)N(C(=O)[C@@H](NC(=O)C3CC3)C3CC3)C[C@@H]21] | Example 9 |
| 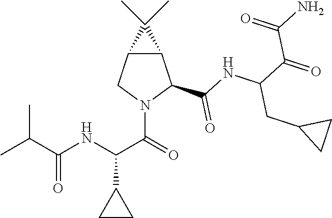 [SMILES: CC(C)C(=O)N[C@H](C(=O)N1C[C@H]2[C@@H]([C@H]1C(=O)NC(CC1CC1)C(=O)C(N)=O)C2(C)C)C1CC1] | Example 10 |
| 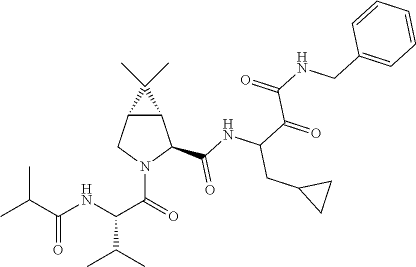 [SMILES: CC(C)C(=O)N[C@H](C(=O)N1C[C@H]2[C@@H]([C@H]1C(=O)NC(CC1CC1)C(=O)C(=O)NCc1ccccc1)C2(C)C)C(C)C] | Example 11 |
| 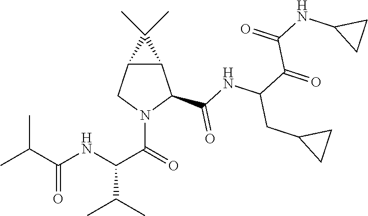 [SMILES: CC(C)C(=O)N[C@H](C(=O)N1C[C@H]2[C@@H]([C@H]1C(=O)NC(CC1CC1)C(=O)C(=O)NC1CC1)C2(C)C)C(C)C] | Example 12 |
| 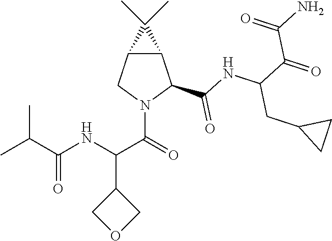 [SMILES: CC(C)C(=O)NC(C(=O)N1C[C@H]2[C@@H]([C@H]1C(=O)NC(CC1CC1)C(=O)C(N)=O)C2(C)C)C1COC1] | Example 13 |
| 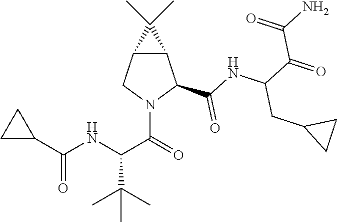 [SMILES: CC(C)(C)[C@H](NC(=O)C1CC1)C(=O)N1C[C@H]2[C@@H]([C@H]1C(=O)NC(CC1CC1)C(=O)C(N)=O)C2(C)C] | Example 14 |
| 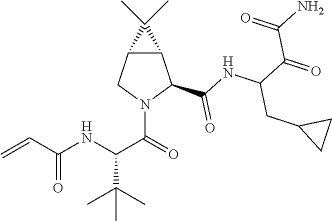 [SMILES: C=CC(=O)N[C@H](C(=O)N1C[C@H]2[C@@H]([C@H]1C(=O)NC(CC1CC1)C(=O)C(N)=O)C2(C)C)C(C)(C)C] | Example 15 |
| 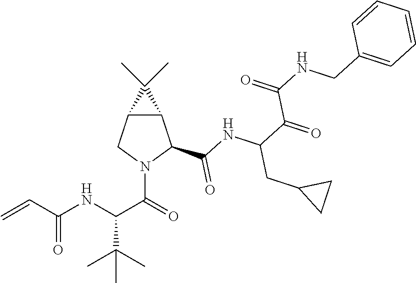 [SMILES: C=CC(=O)N[C@H](C(=O)N1C[C@H]2[C@@H]([C@H]1C(=O)NC(CC1CC1)C(=O)C(=O)NCc1ccccc1)C2(C)C)C(C)(C)C] | Example 16 |
| 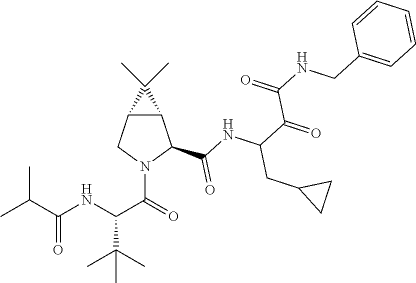 [SMILES: CC(C)C(=O)N[C@H](C(=O)N1C[C@H]2[C@@H]([C@H]1C(=O)NC(CC1CC1)C(=O)C(=O)NCc1ccccc1)C2(C)C)C(C)(C)C] | Example 17 |
| 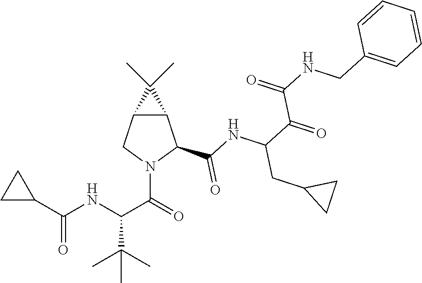 [SMILES: CC(C)(C)[C@H](NC(=O)C1CC1)C(=O)N1C[C@H]2[C@@H]([C@H]1C(=O)NC(CC1CC1)C(=O)C(=O)NCc1ccccc1)C2(C)C] | Example 18 |
| 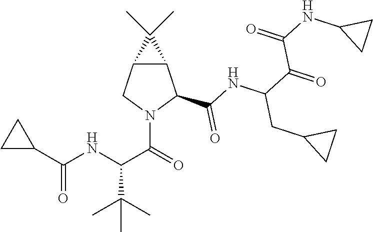 [SMILES: CC(C)(C)[C@H](NC(=O)C1CC1)C(=O)N1C[C@H]2[C@@H]([C@H]1C(=O)NC(CC1CC1)C(=O)C(=O)NC1CC1)C2(C)C] | Example 19 |
| 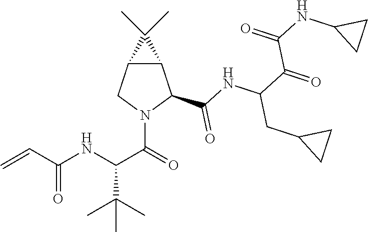 [SMILES: C=CC(=O)N[C@H](C(=O)N1C[C@H]2[C@@H]([C@H]1C(=O)NC(CC1CC1)C(=O)C(=O)NC1CC1)C2(C)C)C(C)(C)C] | Example 20 |
| 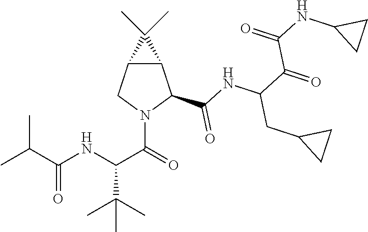 [SMILES: CC(C)C(=O)N[C@H](C(=O)N1C[C@H]2[C@@H]([C@H]1C(=O)NC(CC1CC1)C(=O)C(=O)NC1CC1)C2(C)C)C(C)(C)C] | Example 21 |
| 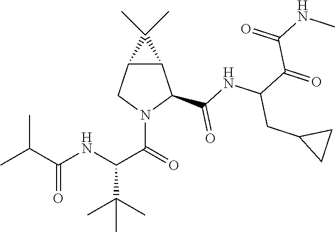 [SMILES: CNC(=O)C(=O)C(CC1CC1)NC(=O)[C@@H]1[C@@H]2[C@H](CN1C(=O)[C@@H](NC(=O)C(C)C)C(C)(C)C)C2(C)C] | Example 22 |
| 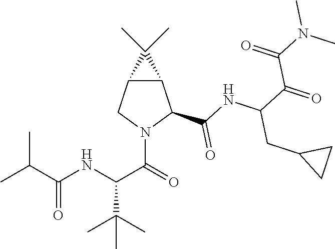 [SMILES: CC(C)C(=O)N[C@H](C(=O)N1C[C@H]2[C@@H]([C@H]1C(=O)NC(CC1CC1)C(=O)C(=O)N(C)C)C2(C)C)C(C)(C)C] | Example 23 |
| 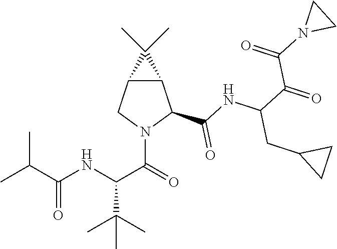 [SMILES: CC(C)C(=O)N[C@H](C(=O)N1C[C@H]2[C@@H]([C@H]1C(=O)NC(CC1CC1)C(=O)C(=O)N1CC1)C2(C)C)C(C)(C)C] | Example 24 |
| 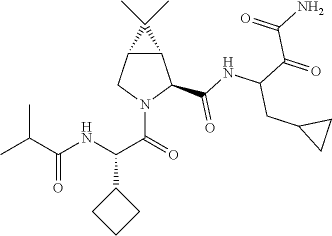 [SMILES: CC(C)C(=O)N[C@H](C(=O)N1C[C@H]2[C@@H]([C@H]1C(=O)NC(CC1CC1)C(=O)C(N)=O)C2(C)C)C1CCC1] | Example 25 |
| 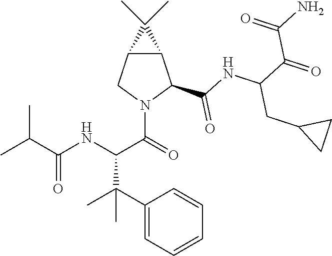 [SMILES: CC(C)C(=O)N[C@H](C(=O)N1C[C@H]2[C@@H]([C@H]1C(=O)NC(CC1CC1)C(=O)C(N)=O)C2(C)C)C(C)(C)c1ccccc1] | Example 26 |
| 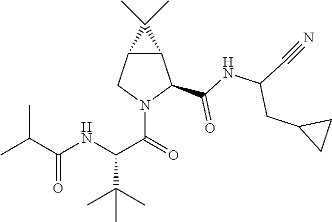 [SMILES: CC(C)C(=O)N[C@H](C(=O)N1C[C@H]2[C@@H]([C@H]1C(=O)NC(C#N)CC1CC1)C2(C)C)C(C)(C)C] | Example 27 |
| 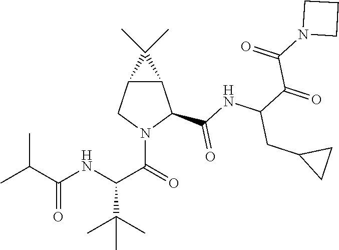 [SMILES: CC(C)C(=O)N[C@H](C(=O)N1C[C@H]2[C@@H]([C@H]1C(=O)NC(CC1CC1)C(=O)C(=O)N1CCC1)C2(C)C)C(C)(C)C] | Example 28 |
| 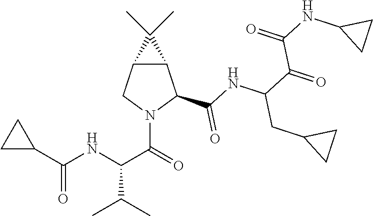 [SMILES: CC(C)[C@H](NC(=O)C1CC1)C(=O)N1C[C@H]2[C@@H]([C@H]1C(=O)NC(CC1CC1)C(=O)C(=O)NC1CC1)C2(C)C] | Example 29 |
| 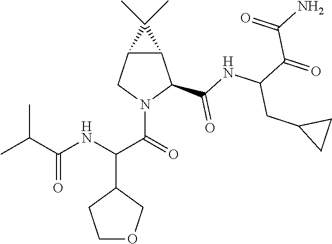 [SMILES: CC(C)C(=O)NC(C(=O)N1C[C@H]2[C@@H]([C@H]1C(=O)NC(CC1CC1)C(=O)C(N)=O)C2(C)C)C1CCOC1] | Example 30 |
| 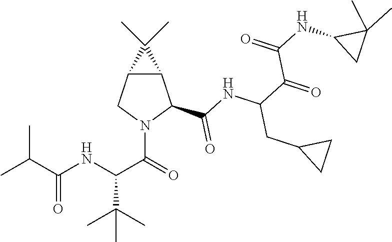 [SMILES: CC(C)C(=O)N[C@H](C(=O)N1C[C@H]2[C@@H]([C@H]1C(=O)NC(CC1CC1)C(=O)C(=O)N[C@H]1CC1(C)C)C2(C)C)C(C)(C)C] | Example 31 |
| 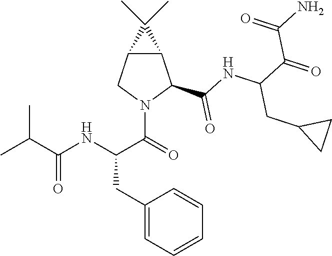 [SMILES: CC(C)C(=O)N[C@@H](Cc1ccccc1)C(=O)N1C[C@H]2[C@@H]([C@H]1C(=O)NC(CC1CC1)C(=O)C(N)=O)C2(C)C] | Example 32 |
| 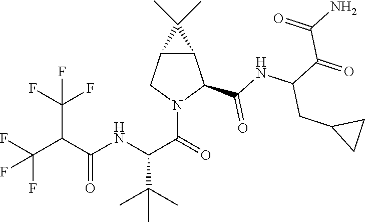 [SMILES: CC(C)(C)[C@H](NC(=O)C(C(F)(F)F)C(F)(F)F)C(=O)N1C[C@H]2[C@@H]([C@H]1C(=O)NC(CC1CC1)C(=O)C(N)=O)C2(C)C] | Example 33 |
| 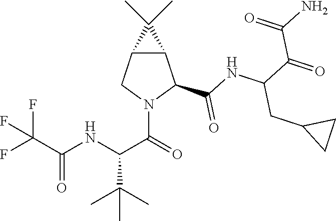 [SMILES: CC(C)(C)[C@H](NC(=O)C(F)(F)F)C(=O)N1C[C@H]2[C@@H]([C@H]1C(=O)NC(CC1CC1)C(=O)C(N)=O)C2(C)C] | Example 34 |
| 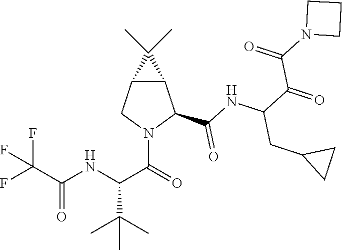 [SMILES: CC(C)(C)[C@H](NC(=O)C(F)(F)F)C(=O)N1C[C@H]2[C@@H]([C@H]1C(=O)NC(CC1CC1)C(=O)C(=O)N1CCC1)C2(C)C] | Example 35 |

### Preparation of the Compounds of the Invention

Compounds of the invention may be prepared by routes including, but not limited to, those detailed in Scheme 1. Details of many of the standard transformations such as those in the routes below and others which could be used to perform the same transformations can be found in standard reference textbooks such as “Organic Synthesis”, M. B. Smith, McGraw-Hill (1994), “Advanced Organic Chemistry”, 4^{th} edition, J. March, John Wiley & Sons (1992) or “Protective Groups in Organic Synthesis”, 3^{rd} edition, T. W. Greene, John Wiley & Sons (1999).

Nitrogen protected derivatives of α-amino acids, for example Boc or Fmoc derivatives, are commercially available, or can be prepared by standard transformations which will be known to those skilled in the art, including transformations that are detailed in the following Synthesis of Intermediates and Synthesis of Examples sections. Similarly, ester derivatives of α-amino acids, for example methyl or ethyl esters, are commercially available, or can be prepared by standard transformations which will be known to those skilled in the art, including transformations that are detailed in the following Synthesis of Intermediates and Synthesis of Examples sections.

The acid functionality in nitrogen protected derivatives of α-amino acids can be coupled with amines to give the corresponding amide derivative (for example Route 1, step vi; Route 2 and 3, step ix; Route 4, step iv), as can other carboxylic acids (for example Route 3, step xi; Route 4, step viii). Amide functionality can also be introduced by other means, for example through the reaction of an amine with ethyl 2,2,2-trifluoroacetate in the presence of a base such as Et₃N, in a solvent such as MeOH, typically at room temperature (for example Route 4, step vii). Derivatives of α-amino acids can be coupled with carboxylic acids to give the corresponding amide derivative (for example Route 1, steps viii, x; Route 2, steps v, vii; Route 3, steps v, vii; Route 4, step ii). The ester functionality present in α-amino acid derivatives, or for example in the product of amide couplings with α-amino acid derivatives, can be hydrolysed under acidic or basic conditions, for example using lithium hydroxide monohydrate in a solvent such as THF, MeOH or H₂O, or a mixture of these solvents, typically at 0° C. or rt (for example Route 2, step viii; Route 4, steps iii, vi). The hydrolysis generates a carboxylic acid that can then be reacted with an amine or a derivative of an α-amino acid, under amide coupling conditions such as those detailed above (for example Route 1, step vi; Route 2 or 3, step ix; Route 3, step xi; Route 4, step viii).

The nitrogen protection present in α-amino acid derivatives, or in the product of amide couplings with α-amino acid derivatives, can be removed under conditions that will be known to those skilled in the art. A Boc group can be removed under acidic conditions, for example using a solution of HCl in EtOAc at rt, TFA in a solvent such as DCM at rt, HCl in a solvent mixture such as 1,4-dioxane/DCM at rt, or concomitantly with another transformation, such as the hydrolysis of a nitrile group to a methyl ester in the presence of TMSCI in MeOH at elevated temperature, such as 60° C. An Fmoc group can be removed under basic conditions, for example 20% piperidine in DMF, at rt. The deprotection reaction yields an amine functionality that can be used in a subsequent amide coupling reaction.

The amide coupling reaction conditions will typically use a coupling agent or agents, for example T3P, HATU, or a combination of agents such as EDCI (often as the hydrochloride salt) and HOBt, with, or without, a suitable base such as DIPEA, NMM or Et₃N, in a solvent such as DCM or DMF, typically at room temperature. Alternatively, derivatives of α-amino acids can be coupled with acid chlorides, for example cyclopropanecarbonyl chloride, with a suitable base such as DIPEA, in a solvent such as DCM, typically at room temperature, to form an amide derivative.

The acid functionality present in α-amino acid derivatives, may be transformed to an aldehyde by methods including the formation of an N-methoxy-N-methylamide derivative (commonly known as a Weinreb amide), and subsequent reduction to the aldehyde using conditions such as the use of lithium aluminium hydride in a solvent such as THF, typically at 0° C. (for example Routes 1, 2 and 3, steps i and ii). The aldehyde can then be used in a sequence of steps to form a substituted, or unsubstituted, ketoamide functionality. For example, in one such reaction, reaction with acetone cyanohydrin in the presence of a base such as Et₃N, in a solvent such as DCM, typically at 0° C. or rt (for example Routes 1 and 3, step iii) yields a 1-cyano, 1-hydroxy derivative. The cyano (also known as nitrile) functionality in the 1-cyano, 1-hydroxy derivative can be hydrolysed to a primary amide, for example using hydrogen peroxide in the presence of a base such as potassium carbonate, in a solvent such as DMSO, typically at 0° C. or rt (for example Route 1, step iv). Alternatively, the cyano functionality may be transformed to a methyl ester by reaction with methanol under acidic conditions, for example in the presence of TMSCI, at an elevated temperature such as 60° C. (for example Route 3, step iv). A similar transformation to an ethyl ester may be effected by reaction of a primary amide group with ethanol and thionyl chloride at an elevated temperature such as 65° C. (for example Route 4, step i). In a second transformation of the aldehyde, reaction with an isocyanide, for example an alkyl or benzyl isocyanide, in the presence of AcOH, in a solvent such as DCM, typically at rt, will yield an alkylamino or benzylamino substituted 1-oxo-2-yl acetate derivative (for example Route 2, step iii). The acetate substitution can be removed under conditions such as Et₃N in MeOH at rt, to yield a substituted hydroxy ketoamide derivative (for example Route 2, step iv). The hydroxy group derived from the aldehyde by the methods above, at this stage, or after further transformations, can be oxidised to a ketone, using an oxidising agent such as IBX or DMP, in a suitable solvent such as DMSO, typically at 0° C. or rt (for example Routes 1, 2 and 3, steps xi, x, xii respectively).

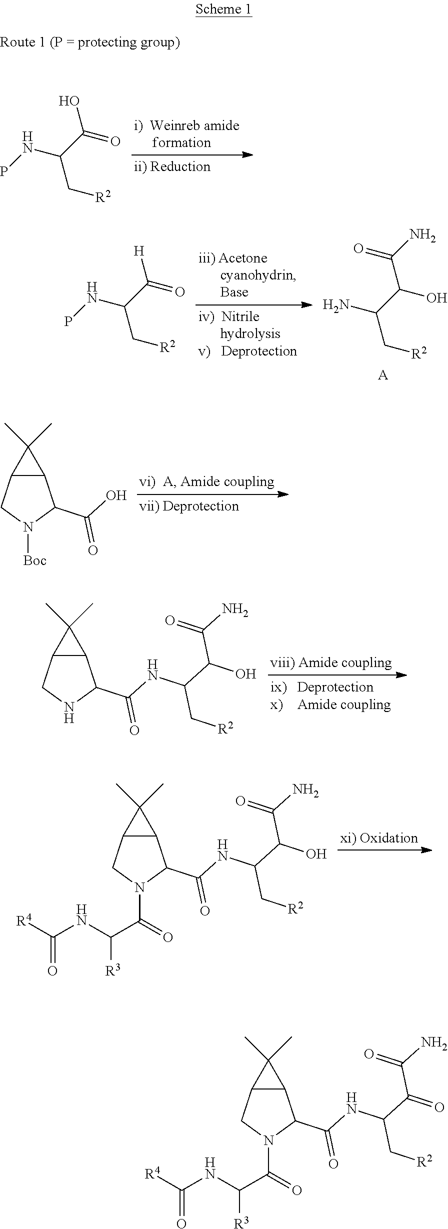

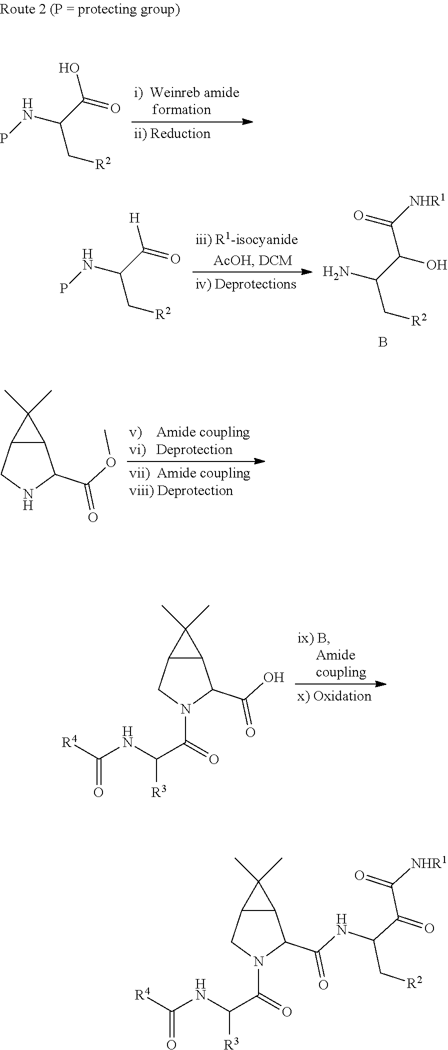

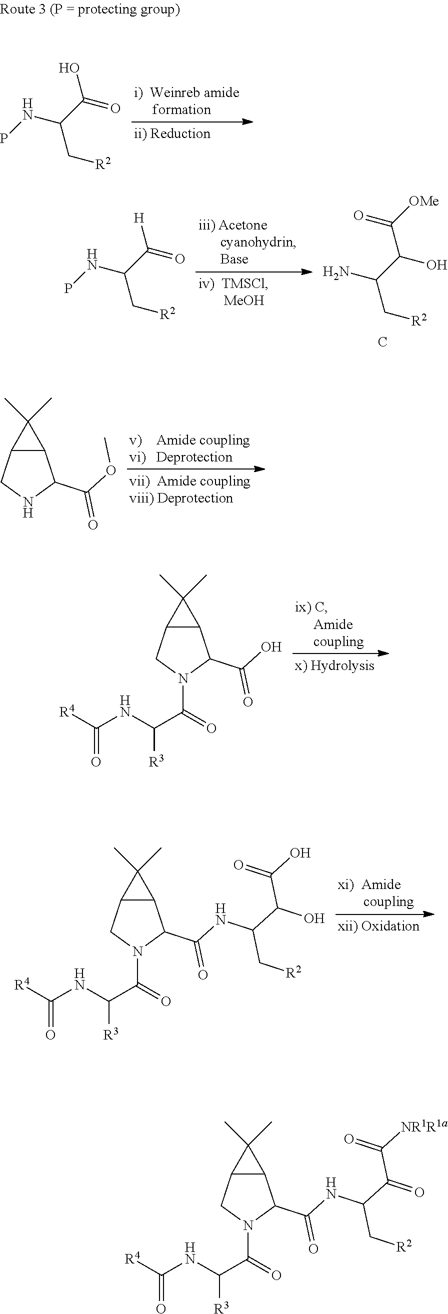

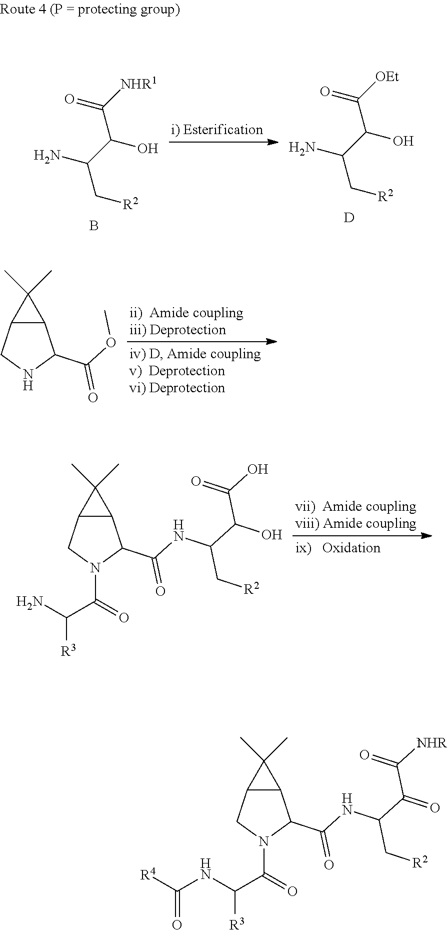

### General Procedures

Where no preparative routes are included, the relevant intermediate is commercially available. Commercial reagents were utilized without further purification. Room temperature (rt) refers to approximately 20-27° C. ¹H NMR spectra were recorded at 300 or 400 MHz on Bruker instruments. Chemical shift values are expressed in parts per million (ppm), i.e. (δ)-values.

The following abbreviations are used for the multiplicity of the NMR signals: s=singlet, br=broad, d=doublet, t=triplet, q=quartet, quin=quintet, h=heptet, dd=doublet of doublets, dt=double of triplets, m=multiplet. Coupling constants are listed as J values, measured in Hz. NMR and mass spectroscopy results were corrected to account for background peaks. TLC for monitoring reactions refers to TLC run using silica gel as a stationary phase.

LCMS experiments were carried out under the following conditions, LCMS data are given in the format: Mass ion, retention time.

Method A. Instrument: Shimadzu LCMS-2020; Column: Chromolith@Flash RP-18E 25-2 MM or Kinetex EVO C18 2.1×30 mm, 5 μm; Gradient [time (min)/solvent B in A (%)]: 0.0/5, 0.8/95, 1.2/95, 1.21/5, 1.55/5 (Solvent A=0.0375% TFA in water (v/v), B=0.01875% TFA in MeCN (v/v)); UV detection 220 and 254 nm; Column temperature 50° C.; Flow rate 1.5 mL/min

Method A2. Instrument: Agilent 1200 LC with Agilent 6110 MSD; Column: Agilent ZORBAX SB-Aq, 2.1*50 mm, 5 μm; Gradient [time (min)/solvent B in A (%)]: 0.0/1, 0.4/1, 3.4/90, 3.91/1, 4.5/1 (Solvent A=0.0375% TFA in water (v/v), B=0.01875% TFA in MeCN (v/v)); UV detection 220 and 254 nm; Column temperature 50° C.; Flow rate 0.8 mL/min

Method B. Instrument: Shimadzu LCMS-2020; Column: Kinetex EVO C18 2.1×30 mm, 5 μm; Method B Gradient [time (min)/solvent B in A (%)]: 0.0/0, 0.8/60, 1.2/60, 1.2/0, 1.55/0 (Solvent A=0.025% NH₃·H₂O (25%, w/w) in water (v/v), B=MeCN); UV detection 210-254 nm; Column temperature 40° C. or 50° C.; Flow rate 0.8 or 1.5 mL/min

Method C. Instrument: Shimadzu LCMS-2020; Column: Chromolith Flash RP-18e 25×2.0 mm or Kinetex EVO C18 2.1×30 mm, 5 μm; Gradient [time (min)/solvent B in A (%)]: 0.0/0, 0.8/60, 1.2/60, 1.21/0, 1.55/0 (Solvent A=0.0375% TFA in water (v/v), B=0.01875% TFA in MeCN (v/v)); UV detection 210-254 nm; Column temperature 50° C.; Flow rate 0.8 mL/min

Method D and D2. Instrument: Agilent G1956A; Column: XBridge C18, 2.1*50 mm, 5 μm; Method D Gradient [time (min)/solvent B in A (%)]: 0.0/5, 0.4/5, 3.4/90, 3.90/100, 3.91/5 (Solvent A=0.05% NH₃·H₂O (25%, w/w) in water (v/v), B=MeCN); Method D2 Gradient [time (min)/solvent B in A (%)]: 0.0/5, 0.4/5, 3.4/90, 4.00/100, 4.01/5 (Solvent A=0.05% NH₃·H₂O (25%, w/w) in water (v/v), B=MeCN); UV detection 220, 254 nm; Column temperature 40° C.; Flow rate 0.8 mL/min

Method E. Instrument: Agilent G1956A; Column: XBridge C18, 2.1*50 mm, 5 μm; Gradient [time (min)/solvent B in A (%)]: 0.0/1, 0.4/1, 3.4/90, 4.00/100, 4.01/1 (Solvent A=0.05% NH₃·H₂O (25%, w/w) in water (v/v), B=MeCN); UV detection 220, 254 nm; Column temperature 40° C.; Flow rate 0.8 mL/min

Method F and F2. Instrument: Method F, Acquity UPLC with PDA & QDA detectors; Method F2; Acquity H-Class with PDA & QDA detectors; Column: C18, 50*2.1 mm, 1.6 μm; Gradient [time (min)/solvent B in A (%)]: 0.0/3, 0.2/3, 2.7/98, 3.00/100, 3.50/100, 3.51/3 (Solvent A=0.1% HCO₂H in water, B=0.1% HCO₂H in water: Acetonitrile (10:90)); Column temperature 35° C.; Flow rate 0.8 mL/min

Method F3. Instrument: Acquity UPLC with PDA & QDA detectors; Column: C18, 50*2.1 mm, 2.5 μm; Gradient [time (min)/solvent B in A (%)]: 0.0/3, 0.2/3, 2.7/98, 3.00/100, 3.50/100, 3.51/3 (Solvent A=0.1% HCO₂H in water, B=0.1% HCO₂H in water: Acetonitrile (10:90)); Column temperature 30° C.; Flow rate 0.8 mL/min

Method G. Instrument: Acquity UPLC with PDA & QDA detectors; Column: C18, 50*2.1 mm, 2.5 μm; Gradient [time (min)/solvent B in A (%)]: 0.0/3, 0.2/3, 2.7/98, 3.0/100, 3.5/100, 3.51/3 (Solvent A=5 mM (NH₄)HCO₃ in water, B=MeCN); Column temperature 35° C.; Flow rate 1.0 mL/min

Method H. Instrument: Waters Alliance 2690 and 996 PDA detector with Micromass ZQ; Column: C18, 150*4.6 mm, 3.5 μm; Gradient [time (min)/solvent B in A (%)]: 0.0/10, 7.0/90, 9.0/100, 14.0/100, 14.01/10 (Solvent A=0.1% TFA in water, B=MeCN); Column temperature ambient; Flow rate 1.0 mL/min

Method I. Instrument: Acquity UPLC with PDA & QDA detectors; Column: C18, 50*2.1 mm, 2.5 μm; Gradient [time (min)/solvent B in A (%)]: 0.0/3, 0.2/3, 2.7/98, 3/100, 3.5/100, 3.51/3 (Solvent A=0.1% HCO₂H in water, B=0.1% HCO₂H in water: Acetonitrile (10:90)); Column temperature 30° C.; Flow rate 0.8 mL/min

Method J. Instrument: Agilent 1100 LC with Agilent G1956A; Column: Waters XBridge C18 2.1*50 mm, 5 μm; Gradient [time (min)/solvent B in A (%)]: 0.0/1, 0.4/1, 3.4/90, 4.0/100, 4.01/1, 4.5/1 (Solvent A=0.05% NH₃·H₂O (25%, w/w) in water (v/v), B=MeCN; UV detection 220 and 254 nm; Column temperature 40° C.; Flow rate 0.8 mL/min

Method K. Instrument: Agilent Infinity II G6125C; Column: C18, 150*4.6 mm, 3.5 μm; Gradient [time (min)/solvent B in A (%)]: 0.0/10, 7.0/90, 9.0/100, 14.0/100, 14.01/10, 17.0/10 (Solvent A=0.1% NH₃ in water (v/v), B=MeCN); Column temperature 25° C.; Flow rate 1.0 mL/min

Method L. Instrument: Agilent 1290 Infinity II Series LC/6125 Quadrupole MSD SL; Column: Waters XBridge C8 50*4.6 mm, 3.5 μm; Gradient [time (min)/solvent B in A (%)]: 0.0/5, 2.5/95, 4.0/95, 4.5/5, 6.0/5. (Solvent A=0.1% TFA in H₂O:MeCN (95:5), B=0.1% TFA in MeCN); UV detection 210 to 400 nm; Column temperature 25° C.; Flow rate 1.5 mL/min.

### Abbreviations

AcOH=acetic acid
Boc=tert-butyloxycarbonyl
CDI=N,N-carbonyldiimidazole
DCM=dichloromethane
DMF=N,N-dimethylformamide
DMP=Dess-Martin Periodinane
DIPEA=N,N-diisopropylethylamine
DMSO=dimethylsulfoxide
EDCI=1-ethyl-3-(3-dimethylaminopropyl)carbodiimide
EtOAc=ethyl acetate
Fmoc=fluorenylmethoxycarbonyl or (((9H-Fluoren-9-yl)methoxy)carbonyl)
h=hour(s)
HATU=1-[bis(dimethylamino)methylene]-1H-1,2,3-triazolo[4,5-b]pyridinium 3-oxide hexafluorophosphate
HOBt=1-hydroxybenzotriazole
HPLC=high performance liquid chromatography
IBX=2-iodoxybenzoic acid
L=litre
LC=liquid chromatography
MeCN=acetonitrile
min=minute(s)
MS=mass spectrometry
NMM=N-methyl morpholine
NMR=nuclear magnetic resonance
MTBE=methyl tert-butyl ether
rt=room temperature
T3P=propylphosphonic anhydride
TFA=trifluoroacetic acid
TFAA=trifluoroacetic anhydride
THF=tetrahydrofuran
TLC=thin layer chromatography
TMSCI=trimethylsilyl chloride
UPLC=ultra performance liquid chromatography

Prefixes n-, s-, i-, t- and tert- have their usual meanings: normal, secondary, iso, and tertiary.

### Synthesis of Intermediates

Intermediate 1: (1 R,2S,5S)—N-[3-Amino-1-(cyclopropylmethyl)-2-hydroxy-3-oxo-propyl]-6,6-dimethyl-3-azabicyclo[3.1.0]hexane-2-carboxamide

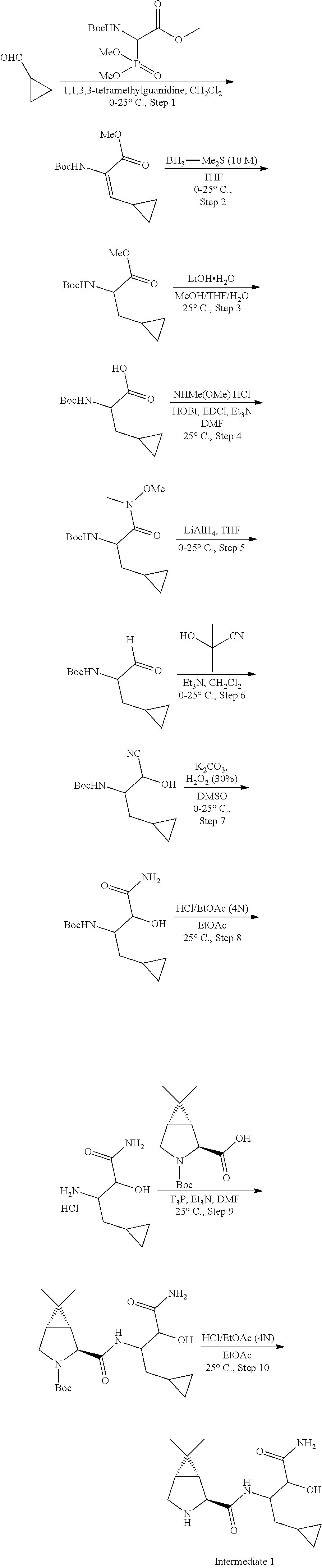

Step 1: To a mixture of methyl 2-(tert-butoxycarbonylamino)-2-dimethoxyphosphoryl acetate (551 g, 1.85 mol) in DCM (2.5 L) was slowly added 1,1,3,3-tetramethylguanidine (214 g, 1.85 mol, 233 mL) at 0° C. After stirring at 0° C. for 30 min, cyclopropanecarbaldehyde (100 g, 1.43 mol, 107 mL) was added to the reaction mixture at 0° C., and the resulting mixture was stirred at 25° C. for 12 h under N₂. H₂O (2 L) was added, the resulting mixture was extracted with DCM (800 mL×3), the combined organic layers were washed with brine (1 L), dried with anhydrous Na₂SO₄, filtered and concentrated in vacuo. Purification by gradient silica gel column chromatography (petroleum ether:EtOAc 1:0 to 10:1) yielded methyl (E)-2-(tert-butoxycarbonylamino)-3-cyclopropyl-prop-2-enoate (374 g, 1.55 mol) as a white solid.

LCMS (Method A): m/z 142.2 (M−100+H), at 0.79 min.

¹H NMR: (400 MHz, DMSO-d₆) δ 0.59-0.64 (2H, m), 0.87-0.93 (2H, m), 1.38 (9H, br s), 1.59-1.71 (1H, m), 3.62 (3H, s), 5.61-5.98 (1H, m), 8.37 (1H, br s).

Step 2: To a solution of methyl (E)-2-(tert-butoxycarbonylamino)-3-cyclopropyl-prop-2-enoate (374 g, 1.55 mol) in THF (2.5 L) at 0° C. under N₂ was slowly added dropwise BH₃-Me₂S (10 M in Me₂S, 620 mL). The reaction mixture was stirred at 25° C. for 24 h under N₂. The reaction mixture was quenched by dropwise addition of MeOH (2 L) at 0° C., the resulting mixture was stirred at 25° C. for 1 h under N₂, then concentrated in vacuo. Purification by gradient silica gel column chromatography (petroleum ether:EtOAc 1:0 to 10:1) yielded methyl 2-(tert-butoxycarbonylamino)-3-cyclopropyl-propanoate (213 g, crude) as a colourless oil.

LCMS (Method A): m/z 144.2 (M−100+H), at 0.83 min.

¹H NMR: (400 MHz, DMSO-d₆) δ −0.05-0.01 (1H, m), 0.07-0.13 (1H, m), 0.35-0.41 (2H, m), 0.73-0.79 (1H, m), 1.33 (1H, br s), 1.38 (9H, s), 1.57-1.64 (1H, m), 3.61 (3H, s), 3.96-4.05 (1H, m), 7.26 (1H, br d, J=7.6 Hz).

Step 3: To a mixture of methyl 2-(tert-butoxycarbonylamino)-3-cyclopropyl-propanoate (213 g, 875 mmol) in MeOH (600 mL), THF (600 mL) and H₂O (600 mL) was added LiOH·H₂O (73.5 g, 1.75 mol) at 25° C. The resulting mixture was stirred at 25° C. for 2 h under N₂ before concentration in vacuo. Aqueous HCl (1N) was added to adjust the pH to approximately 7, and the resulting mixture was extracted with ethyl acetate (600 mL×3). The combined organic layers were washed with brine (800 mL), dried with anhydrous Na₂SO₄, filtered and concentrated in vacuo to yield 2-(tert-butoxycarbonylamino)-3-cyclopropyl-propanoic acid (197 g, crude) as a colorless oil.

LCMS (Method A): m/z 130.2 (M−100+H), at 0.73 min.

¹H NMR: (400 MHz, DMSO-d₆) δ −0.01 (1H, br s), 0.07 (1H, br s), 0.35 (2H, br s), 0.73 (1H, br s), 1.37 (9H, br s), 1.42-1.46 (1H, m), 1.54 (1H, br s), 3.81 (1H, br s), 6.64 (1H, br s).

Step 4: To a mixture of 2-(tert-butoxycarbonylamino)-3-cyclopropyl-propanoic acid (197 g, 859 mmol) in DMF (800 mL) was slowly added HOBt (139 g, 1.03 mol) and EDCl·HCl (198 g, 1.03 mol) at 25° C. After stirring at 25° C. for approximately 12 min, N-methoxymethanamine hydrochloride (101 g, 1.03 mol) and Et₃N (104 g, 1.03 mol, 144 mL) were added and the resulting mixture was stirred at 25° C. for 2 h under N₂. H₂O (1.5 L) was added and the resulting mixture was extracted with EtOAc (800 mL×3). The combined organic layers were washed with brine (500 mL×3), dried with anhydrous Na₂SO₄, filtered and concentrated in vacuo. Purification by gradient silica gel column chromatography (petroleum ether:EtOAc 1:0 to 10:1) yielded tert-butyl-N-[1-(cyclopropylmethyl)-2-[methoxy(methyl)amino]-2-oxo-ethyl]carbamate (209 g, 0.77 mol) as a white solid.

LCMS (Method A): m/z 173.2 (M−100+H), at 0.81 min.

¹H NMR: (400 MHz, DMSO-d₆) δ −0.06-0.01 (1H, m), 0.07-0.13 (1H, m), 0.35-0.41 (2H, m), 0.74-0.78 (1H, m), 1.14-1.22 (1H, m), 1.37 (9H, s), 1.58-1.66 (1H, m), 3.09 (3H, s), 3.73 (3H, s), 4.44 (1H, br d, J=3.2 Hz), 7.01 (1H, br d, J=8.4 Hz).

Step 5: To a mixture of tert-butyl N-[1-(cyclopropylmethyl)-2-[methoxy(methyl)amino]-2-oxo-ethyl]carbamate (189 g, 659 mmol) in THF (1.5 L) was slowly added LiAlH₄ (25.0 g, 659 mmol) at 0° C., and the reaction mixture was stirred at 25° C. for 1 h under N₂. H₂O (25 mL) was added dropwise at 0° C., the resulting mixture was filtered and the residue washed with THF (1 L). The filtrate was dried with anhydrous Na₂SO₄, filtered and concentrated in vacuo. The residue was purified by gradient silica gel column chromatography (petroleum ether:EtOAc 1:0 to 5:1) to yield tert-butyl N-[1-(cyclopropylmethyl)-2-oxo-ethyl]carbamate (105 g, crude) as a light yellow oil.

LCMS (Method A): m/z 114.2 (M−100+H), at 0.75 min.

¹H NMR: (400 MHz, DMSO-d₆) δ 0.06-0.08 (2H, m), 0.38-0.40 (2H, m), 0.76-0.78 (1H, m), 1.40 (9H, s), 1.44-1.48 (2H, m), 3.86-3.92 (1H, m), 7.31 (1H, br d, J=7.2 Hz), 9.45 (1H, s).

Step 6: To a mixture of tert-butyl N-[1-(cyclopropylmethyl)-2-oxo-ethyl]carbamate (121 g, 567 mmol) in DCM (1 L) was slowly added Et₃N (86.1 g, 851 mmol, 118 mL) and 2-hydroxy-2-methyl-propanenitrile (74.7 g, 878 mmol, 80.2 mL) at 0° C. The reaction mixture was stirred at 25° C. for 12 h under N₂. The reaction mixture was quenched by addition of saturated aqueous NaHCO₃ (500 mL) solution at 0° C., and the resulting mixture extracted with DCM (300 mL×3). The combined organic layers were washed with brine (500 mL), dried with anhydrous Na₂SO₄, filtered and concentrated in vacuo. Purification by gradient silica gel column chromatography (petroleum ether:EtOAc 1:0 to 5:1) yielded tert-butyl N-[2-cyano-1-(cyclopropylmethyl)-2-hydroxy-ethyl]carbamate (69.2 g, crude) as a light yellow oil.

¹H NMR: (400 MHz, DMSO-d₆) δ −0.06-0.01 (1H, m), 0.09-0.15 (1H, m), 0.35-0.41 (2H, m), 0.66-0.75 (1H, m), 1.33-1.35 (1H, m), 1.39 (9H, s), 1.48-1.56 (1H, m), 3.60-3.71 (1H, m), 6.49-6.60 (1H, m), 6.93-7.07 (1H, m).

Step 7: To a mixture of tert-butyl N-[2-cyano-1-(cyclopropylmethyl)-2-hydroxy-ethyl]carbamate (71.0 g, 295 mmol) and K₂CO₃ (81.7 g, 591 mmol) in DMSO (500 mL) was slowly added dropwise H₂O₂(449 g, 3.96 mol, 380 mL, 30% purity, w/w) at 0° C. The reaction mixture was stirred at 0-25° C. for 12 h under N₂. The reaction mixture was diluted with H₂O (1 L) and slowly quenched by addition of saturated aqueous Na₂S₂O₃ (1 L) solution at 0° C. The resulting mixture was acidified to approximately pH 8 with aqueous 1 N HCl solution, then extracted with DCM (500 mL×3). The combined organic layers were washed with brine (500 mL), and saturated aqueous Na₂S₂O₃ (500 mL) solution, dried with anhydrous Na₂SO₄, filtered and concentrated in vacuo. Purification by gradient silica gel column chromatography (petroleum ether:EtOAc 10:1 to 0:1) yielded tert-butyl N-[3-amino-1-(cyclopropylmethyl)-2-hydroxy-3-oxo-propyl]carbamate (35.6 g, 0.14 mol).

LCMS (Method A): m/z 159.2 (M−100+H), at 0.70 min.

¹H NMR: (400 MHz, DMSO-d₆) δ 0.01-0.05 (2H, m), 0.37 (2H, br d, J=7.6 Hz), 0.63-0.67 (1H, m), 1.36 (9H, s), 1.39 (2H, s), 3.77-3.82 (1H, m), 3.88-3.91 (1H, m), 5.44 (1H, d, J=6.4 Hz), 5.95 (1H, br d, J=9.2 Hz), 7.20 (2H, br d, J=10.8 Hz).

Step 8: HCl/EtOAc (4 N, 34 mL) was added to a mixture of tert-butyl N-[3-amino-1-(cyclopropylmethyl)-2-hydroxy-3-oxo-propyl]carbamate (5.60 g, 21.7 mmol) in EtOAc (30 mL) at 25° C. The resulting mixture was stirred at 25° C. for 1 h under N₂. After concentration in vacuo purification by preparative HPLC (HCl as additive) yielded 3-amino-4-cyclopropyl-2-hydroxy-butanamide hydrochloride (3.30 g, 20.9 mmol) as a white solid.

LCMS (Method B): m/z 158.9 (M+H), at 0.38 min.

¹H NMR: (400 MHz, DMSO-d₆) δ −0.06-0.13 (2H, m), 0.37-0.46 (2H, m), 0.74-0.85 (1H, m), 1.21-1.51 (2H, m), 3.45-3.47 (1H, m), 4.11-4.28 (1H, m), 6.18-6.57 (1H, m), 7.42-7.55 (2H, m), 7.95 (1H, br s), 8.25 (1H, br s).

Preparative HPLC (HCl) method for the purification of Step 8. Instrument: Shimadzu LC-20AP; Column: Phenomenex luna C18 250×80 mm×10 μm; Mobile phase: A=0.05% HCl in water (v/v), B=MeCN; Gradient: 0-10% B in A over 15 min; Flow rate 150 mL/min; Column temperature: 40° C.; Wavelength: 220 nm, 254 nm.

Step 9: To a mixture of 3-amino-4-cyclopropyl-2-hydroxy-butanamide (2.00 g, 12.6 mmol) and (1 R,2S,5S)-3-tert-butoxycarbonyl-6,6-dimethyl-3-azabicyclo[3.1.0]hexane-2-carboxylic acid (2.97 g, 11.6 mmol) in DMF (30 mL) was slowly added Et₃N (2.56 g, 25.3 mmol, 3.52 mL) and T3P (12.1 g, 19.0 mmol, 11.3 mL, 50% purity in EtOAc) at 25° C. The resulting mixture was stirred at 25° C. for 2 h under N₂ before addition of H₂O (50 mL) and extraction with EtOAc (50 mL×3). The combined organic layers were washed with brine (50 mL), dried with anhydrous Na₂SO₄, filtered and concentrated in vacuo. Purification by gradient silica gel column chromatography (petroleum ether:EtOAc 10:1 to 1:1), then by preparative HPLC (NH₄HCO₃ as additive) yielded tert-butyl(1 R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carboxylate (1.43 g, 3.62 mmol) as a white solid.

LCMS (Method C): m/z 396.2 (M+H), at 0.99 min.

¹H NMR: (400 MHz, DMSO-d₆) δ −0.15-0.11 (2H, m), 0.23-0.41 (2H, m), 0.62-0.76 (1H, m), 0.83-0.87 (1H, m), 0.87-1.03 (6H, m), 1.27-1.36 (9H, m), 1.36-1.40 (2H, m), 1.42-1.62 (1H, m), 3.23-3.31 (1H, m), 3.43-3.58 (1H, m), 3.83-4.02 (2H, m), 4.03-4.23 (1H, m), 5.43-5.88 (1H, m), 7.12-7.29 (2H, m), 7.39-8.06 (1H, m).

Preparative HPLC (NH₄HCO₃) method for the purification of Step 9. Instrument: Shimadzu LC-20AP; Column: Kromasil Eternity XT 250×80 mm×10 μm; Mobile phase: A=10 mM aqueous NH₄HCO₃ solution (v/v), B=MeCN; Gradient: 37-67% B in A over 20 min; Flow rate: 140 mL/min; Column temperature: 40° C.; Wavelength: 220 nm, 254 nm.

Step 10: To a mixture of tert-butyl (1 R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carboxylate (1.40 g, 3.24 mmol) in EtOAc (5 mL) was added HCl/EtOAc (4 N, 15.3 mL) at 25° C., and the resulting mixture stirred at 25° C. for 1 h under N₂. Concentration in vacuo yielded (1R,2S,5S)—N-[3-amino-1-(cyclopropylmethyl)-2-hydroxy-3-oxo-propyl]-6,6-dimethyl-3-azabicyclo[3.1.0]hexane-2-carboxamide (Intermediate 1, 1.15 g, 3.89 mmol) as a light yellow solid.

LCMS (Method C): m/z 296.1 (M+H), at 0.66 min.

¹H NMR: (400 MHz, DMSO-d₆) δ −0.16-0.17 (2H, m), 0.27-0.44 (2H, m), 0.60-0.72 (1H, m), 1.02-1.09 (6H, m), 1.29-1.64 (2H, m), 1.65-1.89 (2H, m), 3.02-3.07 (1H, m), 3.60 (2H, br s), 3.89-3.98 (1H, m), 4.18-4.29 (1H, m), 7.08-7.38 (2H, m), 8.35-8.72 (1H, m), 8.72-8.94 (1H, m), 9.87-10.23 (1H, m).

### Intermediate 2: (1R,2S,5S)—N-[3-Amino-1-(cyclopropylmethyl)-2-hydroxy-3-oxo-propyl]-3-[(2S)-2-amino-3-methyl-butanoyl]-6,6-dimethyl-3-azabicyclo[3.1.0]hexane-2-carboxamide

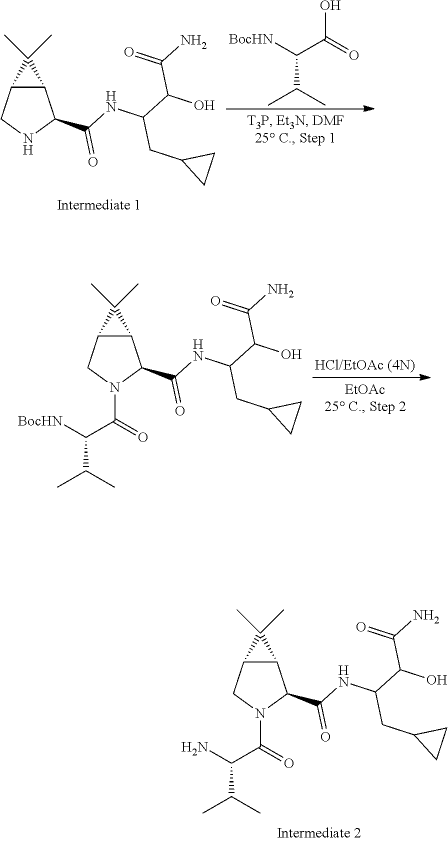 [SMILES: CC1(C)[C@@H]2[C@@H](C(=O)NC(CC3CC3)C(O)C(N)=O)NC[C@@H]21 ; CC(C)[C@H](NC(=O)OC(C)(C)C)C(=O)O ; CC(C)[C@H](NC(=O)OC(C)(C)C)C(=O)N1C[C@H]2[C@@H]([C@H]1C(=O)NC(CC1CC1)C(O)C(N)=O)C2(C)C ; CC(C)[C@H](N)C(=O)N1C[C@H]2[C@@H]([C@H]1C(=O)NC(CC1CC1)C(O)C(N)=O)C2(C)C]

Step 1: To a mixture of (1R,2S,5S)—N-[3-amino-1-(cyclopropylmethyl)-2-hydroxy-3-oxo-propyl]-6,6-dimethyl-3-azabicyclo[3.1.0]hexane-2-carboxamide Intermediate 1 (280 mg, 0.95 mmol) and (tert-butoxycarbonyl)-L-valine (309 mg, 1.42 mmol) in DMF (3 mL) were slowly added Et₃N (192 mg, 1.90 mmol, 0.26 mL) and T3P (905 mg, 1.42 mmol, 0.85 mL, 50% purity, w/w) at 25° C. The resulting mixture was stirred at 25° C. for 12 h under N₂. H₂O (50 mL) was added and the resulting mixture was extracted with EtOAc (20 mL×3). The combined organic layers were washed with brine (30 mL), dried with anhydrous Na₂SO₄, filtered and concentrated in vacuo. The residue was purified by preparative HPLC (formic acid as additive) to yield tert-butyl N-[(1S)-1-[(1R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carbonyl]-2-methyl-propyl]carbamate (183 mg, 0.37 mmol) as a white solid.

LCMS (Method C): m/z 495.2 (M+H), at 1.04 min.

¹H NMR: (400 MHz, DMSO-d₆) δ −0.15-0.08 (2H, m), 0.26-0.41 (2H, m), 0.63-0.72 (1H, m), 0.76-0.99 (12H, m), 1.00 (2H, br s), 1.14-1.30 (1H, m), 1.30-1.39 (9H, m), 1.40-1.45 (1H, m), 1.77-1.95 (1H, m), 3.70-3.84 (2H, m), 3.85-3.97 (2H, m), 4.04-4.29 (2H, m), 4.98-5.96 (1H, m), 6.88-7.00 (1H, m), 7.12-7.28 (2H, m), 7.37-7.81 (1H, m).

Preparative HPLC (formic acid) method for the purification of Step 1. Instrument: Shimadzu LC-20AP; Column: Phenomenex luna C18 150×40 mm×15 μm; Mobile phase: A=0.225% formic acid in water (v/v), B=MeCN; Gradient: 33-63% B in A over 10 min; Flow rate: 60 mL/min; Column temperature: 40° C.; Wavelength: 220 nm, 254 nm.

Step 2: HCl/EtOAc (4 N, 3 mL) was added to a mixture of tert-butyl N-[(1S)-1-[(1R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carbonyl]-2-methyl-propyl]carbamate (150 mg, 0.30 mmol) in EtOAc (3 mL) at 25° C., and the resulting mixture was stirred at 25° C. for 1 h under N₂. The reaction mixture was concentrated in vacuo to yield (1 R,2S,5S)—N-[3-amino-1-(cyclopropylmethyl)-2-hydroxy-3-oxo-propyl]-3-[(2S)-2-amino-3-methyl-butanoyl]-6,6-dimethyl-3-azabicyclo[3.1.0]hexane-2-carboxamide (125 mg, crude) as a light yellow solid.

LCMS (Method C): m/z 395.2 (M+H), at 0.73 min.

### Intermediate 3: (1 R,2S,5S)—N-(4-Amino-3-hydroxy-1-((1 R,2S)-2-methylcyclopropyl)-4-oxobutan-2-yl)-6,6-dimethyl-3-azabicyclo[3.1.0]hexane-2-carboxamide

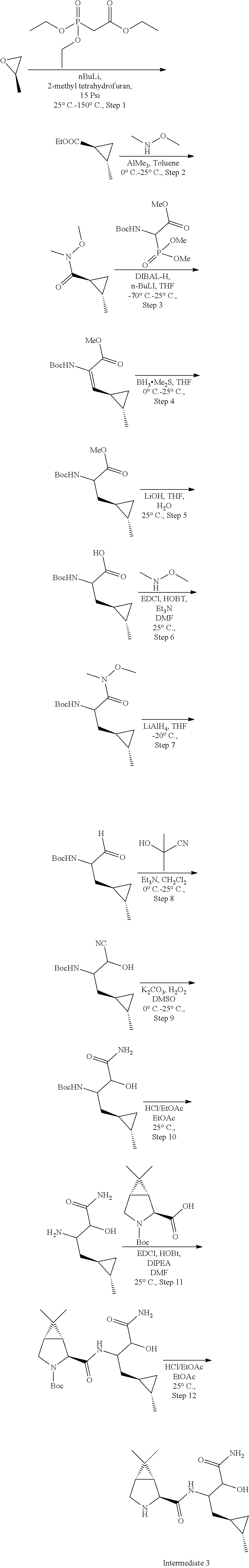

Step 1: n-BuLi (2.5M in n-hexane, 344 mL) was added dropwise to a solution of ethyl 2-diethoxyphosphorylacetate (193 g, 861 mmol, 171 mL) in 2-methyl tetrahydrofuran (1 L) at 25° C. under N₂. After the resulting mixture was stirred at 25° C. for 30 min (2R)-2-methyloxirane (50.0 g, 861 mmol, 60.3 mL) was added at 25° C. The resulting mixture was stirred at 150° C. for 12 h in a 5 L autoclave at a pressure of 15 Psi. The reaction mixture was cooled to 25° C. and distillation at 100° C. under reduced pressure (approximately 0.03 bar) yielded step 1 product, (2S)-2-methylcyclopropanecarboxylate (91.0 g, 710 mmol) as a colourless oil.

¹H NMR: (400 MHz, CDCl₃) δ 0.63-0.69 (1H, m), 1.11 (3H, d, J=6.0 Hz), 1.13-1.18 (1H, m), 1.24-1.28 (3H, m), 1.34-1.44 (2H, m), 4.07-4.15 (2H, m).

Step 2: AlMe₃ (2M in PhMe, 78.0 mL, 156.0 mmol) was added dropwise to a mixture of N-methoxymethanamine hydrochloride (15.2 g, 156 mmol) in PhMe (100 mL) at 0° C. under N₂. The resulting mixture was stirred at 25° C. for 30 min, then a solution of ethyl (2S)-2-methylcyclopropanecarboxylate (10.0 g, 78.0 mmol) in PhMe (150 mL) was added at 0° C. The resulting mixture was stirred at 25° C. for 12 h before the addition of H₂O (100 mL). The aqueous phase was extracted with ethyl acetate (100 mL×3) and combined organic phases were washed with brine (100 mL×3), dried (Na₂SO₄), filtered and concentrated in vacuo. The residue was purified by gradient silica gel column chromatography, eluting with petroleum ether:ethyl acetate 20:1 to 5:1, to yield (1 S,2S)—N-methoxy-N,2-dimethyl-cyclopropanecarboxamide (3.00 g, 21.0 mmol) as a yellow oil.

LCMS (Method A): m/z 144.2 (M+H), at 0.60 min.

¹H NMR: (400 MHz, CDCl₃) δ 0.61-0.68 (1H, m), 1.13 (3H, d, J=6.0 Hz), 1.16-1.21 (1H, m), 1.31-1.41 (1H, m), 1.78-1.90 (1H, m), 3.20 (3H, s), 3.75 (3H, s).

Step 3: To a mixture of (1 S,2S)—N-methoxy-N,2-dimethyl-cyclopropanecarboxamide (3.00 g, 21.0 mmol) in THF (30 mL) was added DIBAL-H (1 M in PhMe, 23.1 mL, 23.1 mmol) dropwise at −70° C. under N₂. The resulting mixture was stirred at −70° C. for 30 min. Separately, n-BuLi (2.5M in n-hexane, 17.6 mL, 35.2 mmol) was added dropwise to a solution of methyl 2-(tert-butoxycarbonylamino)-2-dimethoxyphosphoryl acetate (6.85 g, 23.1 mmol) in THF (70 mL) at −70° C. under N₂, and the resulting mixture was stirred at −70° C. for 30 min, before adding to the first reaction mixture at −70° C. After stirring at 25° C. for 12 h water (100 mL) was added at 0° C. and the resulting mixture was extracted with EtOAc (100 mL×3). The combined organic layers were dried over anhydrous Na₂SO₄, filtered and concentrated in vacuo. Purification by gradient silica gel column chromatography, eluting with petroleum ether/ethyl acetate 10:1 to 5:1 yielded methyl (E)-2-(tert-butoxycarbonylamino)-3-[(1 S,2S)-2-methylcyclopropyl]prop-2-enoate (3.40 g, 13.3 mmol) as a yellow oil.

LCMS (Method A): m/z 200.1 (M-56+H), at 0.85 min.

¹H NMR: (400 MHz, CDCl₃) δ 0.77-0.85 (2H, m), 1.04-1.11 (1H, m), 1.12-1.17 (3H, m), 1.35-1.42 (1H, m), 1.48 (9H, s), 3.75 (3H, s), 5.74-5.96 (1H, m), 6.06 (1H, d, J=10.8 Hz).

Steps 4-12: (1 R,2S,5S)—N-(4-Amino-3-hydroxy-1-((1 R,2S)-2-methylcyclopropyl)-4-oxobutan-2-yl)-6,6-dimethyl-3-azabicyclo[3.1.0]hexane-2-carboxamide hydrochloride (Intermediate 3, 0.39 g, 1.26 mmol, light yellow solid) was formed from methyl (E)-2-(tert-butoxycarbonylamino)-3-[(1 S,2S)-2-methylcyclopropyl]prop-2-enoate (23.5 g, 92.1 mmol) using similar procedures to those detailed for Intermediate 1 (Steps 2-10).

LCMS (Method B): m/z 310.2 (M+H), at 1.00 min.

¹H NMR: (400 MHz, CD₃OD) δ 0.10-0.61 (4H, m), 0.94-1.05 (3H, m), 1.11-1.19 (6H, m), 1.21-1.49 (2H, m), 1.52-1.70 (1H, m), 1.78-1.85 (1H, m), 3.21-3.30 (1H, m), 3.71-3.79 (1H, m), 4.08-4.20 (2H, m), 4.29-4.48 (1H, m).

### Intermediate 4: Isobutyryl-L-valine

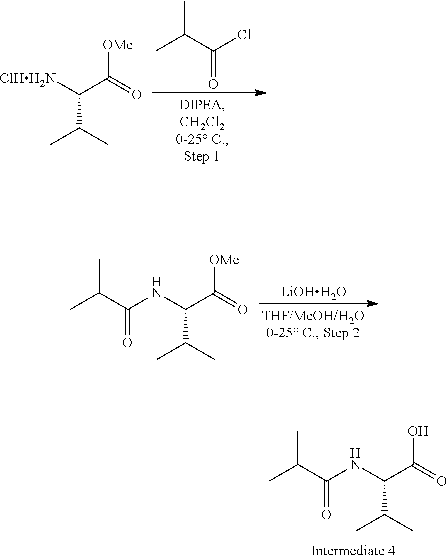

Step 1: To a mixture of methyl (2S)-2-amino-3-methyl-butanoate hydrochloride (5.00 g, 29.8 mmol) in DCM (50 mL) was added DIPEA (11.6 g, 89.5 mmol, 15.6 mL) and 2-methylpropanoyl chloride (3.81 g, 35.8 mmol) at 0° C. The resulting mixture was stirred at 25° C. for 12 h before the addition of DCM (100 mL) and water (80 mL). The organic phase was washed with water (2×80 mL), dried over anhydrous Na₂SO₄, filtered and concentrated in vacuo to yield methyl (2S)-3-methyl-2-(2-methylpropanoylamino)butanoate (6.00 g, 29.8 mmol) as a yellow oil.

LCMS (Method A): m/z 202.2 (M+H), at 0.35 min.

Step 2: LiOH·H₂O (2.50 g, 59.6 mmol) was added to a mixture of methyl (2S)-3-methyl-2-(2-methylpropanoylamino)butanoate (4.00 g, 19.9 mmol) in THF (20 mL), MeOH (6 mL) and H₂O (6 mL) at 0° C., and the resulting mixture stirred at 25° C. for 1 h. Aqueous 2N HCl solution was added to adjust to approximately pH 7, followed by EtOAc (100 mL). The phases were separated, the organic phase was washed with water (80 mL×3), dried over anhydrous Na₂SO₄, filtered and concentrated in vacuo. Purification by preparative HPLC (formic acid as additive) yielded (2S)-3-methyl-2-(2-methylpropanoylamino)butanoic acid (Intermediate 4, 1.60 g, 8.55 mmol) as a white solid.

LCMS (Method A): m/z 188.2 (M+H), at 0.37 min.

¹H NMR: (400 MHz, DMSO-d₆) δ 0.96-0.99 (6H, m), 1.11-1.14 (6H, m), 2.12-2.20 (1H, m), 2.56-2.64 (1H, m), 4.00 (1H, d, J=6.0 Hz).

Preparative HPLC (formic acid) method for the purification of Intermediate 4. Instrument: Shimadzu LC-20AP; Column: Phenomenex Synergi Max-RP C18 250×50 mm×10 μm; Mobile phase: A=0.225% formic acid in water (v/v), B=MeCN; Gradient: 1-30% B in A over 20 min; Flow rate: 150 mL/min; Column temperature: 40° C.; Wavelength: 220 nm, 254 nm.

### Intermediate 5: (1 R,2S,5S)-3-(Isobutyryl-L-valyl)-6,6-dimethyl-3-azabicyclo[3.1.0]hexane-2-carboxylic acid

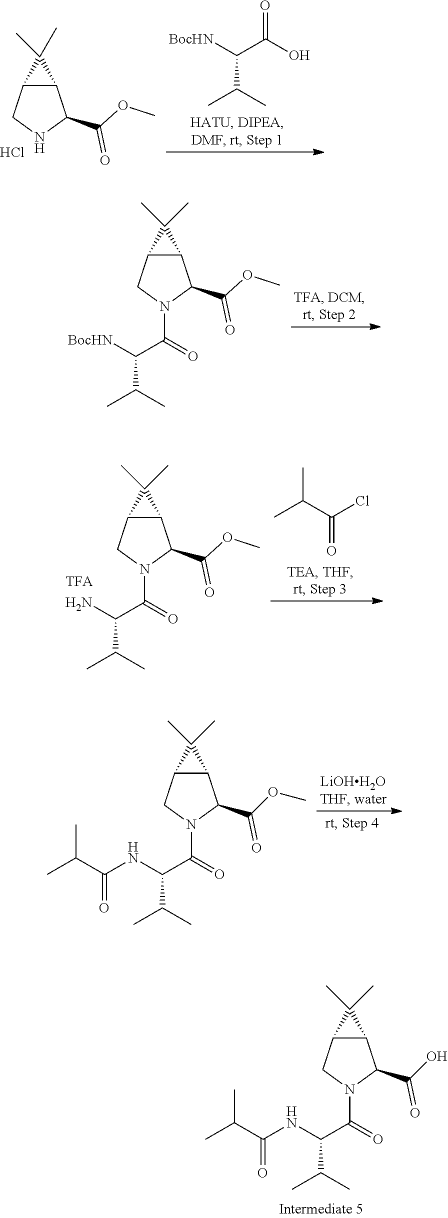

Step 1: HATU (10.5 g, 27.7 mmol) was added to a solution of (tert-butoxycarbonyl)-L-valine (5.00 g, 23.0 mmol) in DMF (30 mL) and the mixture was stirred at rt for 10 min. Methyl (1 R,2S,5S)-6,6-dimethyl-3-azabicyclo[3.1.0]hexane-2-carboxylate hydrochloride (5.66 g, 27.7 mmol) and DIPEA (11.9 mL, 69.1 mmol) were added and the reaction mixture was stirred at rt for 2 h. The reaction mixture was partitioned between cold water (250 mL) and EtOAc (100 mL), the aqueous phase was extracted with EtOAc (2×70 mL) and the combined organic phases were dried (Na₂SO₄). After concentration in vacuo purification by reverse phase gradient flash column chromatography on C18 silica, eluting with 0-50% MeCN in water yielded methyl (1 R,2S,5S)-3-((tert-butoxycarbonyl)-L-valyl)-6,6-dimethyl-3-azabicyclo[3.1.0]hexane-2-carboxylate (5.90 g, 16.0 mmol) as a yellow sticky solid.

LCMS (Method F): m/z 269.3 (M-100), at 2.24 min.

Step 2: TFA (6 mL) was added drop wise to a solution of methyl (1 R,2S,5S)-3-((tert-butoxycarbonyl)-L-valyl)-6,6-dimethyl-3-azabicyclo[3.1.0]hexane-2-carboxylate (5.90 g, 16.0 mmol) in DCM (50 mL) at 0° C. After stirring at rt for 2 h, concentration in vacuo yielded methyl (1R,2S,5S)-3-(L-valyl)-6,6-dimethyl-3-azabicyclo[3.1.0]hexane-2-carboxylate trifluoroacetate (6.50 g) as a yellow sticky solid.

LCMS (Method F): m/z 269.3 (M+H), at 0.93 min.

Step 3: Et₃N (7.17 mL, 51.0 mmol) was added dropwise to a solution of methyl (1R,2S,5S)-3-(L-valyl)-6,6-dimethyl-3-azabicyclo[3.1.0]hexane-2-carboxylate trifluoroacetate (6.50 g, 17.0 mmol) in THF (100 mL) at 0° C. and the reaction mixture was stirred at 0° C. for 10 min. Isobutyryl chloride (1.80 mL, 17.0 mmol) was added, and after stirring at rt for 1 h water (250 mL) and EtOAc (100 mL) were added and the phases were separated. The aqueous layer was extracted with EtOAc (2×100 mL), the combined organic layers were dried (Na₂SO₄) and concentrated in vacuo. Purification by reverse phase gradient flash column chromatography on C18 silica, eluting with 0-40% MeCN in water yielded methyl (1R,2S,5S)-3-(isobutyryl-L-valyl)-6,6-dimethyl-3-azabicyclo[3.1.0]hexane-2-carboxylate (4.10 g, 12.1 mmol) as a white solid.

LCMS (Method F): m/z 339.3 (M+H), at 1.79 min.

Step 4: LiOH·H₂O (2.48 g, 60.6 mmol) was added to a solution of methyl (1R,2S,5S)-3-(isobutyryl-L-valyl)-6,6-dimethyl-3-azabicyclo[3.1.0]hexane-2-carboxylate (4.10 g, 12.1 mmol) in THF (20 mL) and water (10 mL) and the mixture stirred at rt for 2 h. Water (50 mL) was added and the mixture acidified with glacial AcOH (20 mL) to approximately pH 5. The aqueous layer was extracted with 10% MeOH in DCM (3×200 mL). The combined organic layers were dried (Na₂SO₄) and concentrated in vacuo to yield (1 R,2S,5S)-3-(isobutyryl-L-valyl)-6,6-dimethyl-3-azabicyclo[3.1.0]hexane-2-carboxylic acid (Intermediate 5, 3.30 g, 10.2 mmol) as a white solid that was used without further purification.

LCMS (Method F): m/z 325.3 (M+H), at 1.49 min.

### Intermediate 6: 3-Amino-N-benzyl-4-cyclopropyl-2-hydroxybutanamide

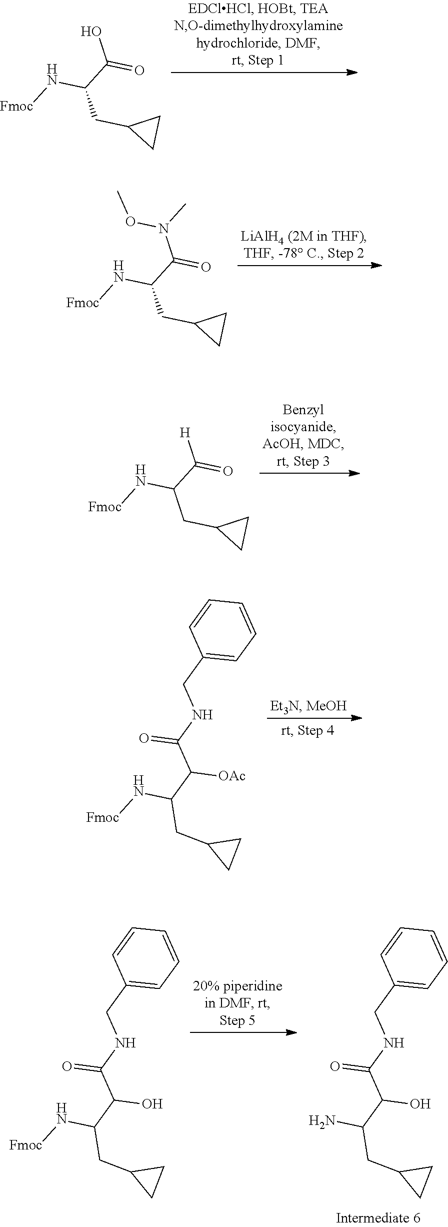

Step 1: (S)-2-((((9H-Fluoren-9-yl)methoxy)carbonyl)amino)-3-cyclopropylpropanoic acid (12.0 g, 34.2 mmol), EDCl·HCl (7.20 g, 37.5 mmol) and HOBt (5.06 g, 37.5 mmol) were dissolved in DMF (20 mL) at rt and stirred for 20 mins. N,O-Dimethylhydroxylamine hydrochloride (3.65 g, 37.5 mmol) and Et₃N (5.26 mL, 37.5 mmol) were added and the reaction mixture was stirred at rt for 2 h. After partitioning between water (200 mL) and EtOAc (100 mL) the aqueous layer was extracted with EtOAc (2×70 mL). The combined organic layers were dried (Na₂SO₄) and concentrated in vacuo. Trituration with n-hexane yielded (9H-fluoren-9-yl)methyl (S)-(3-cyclopropyl-1-(methoxy(methyl)amino)-1-oxopropan-2-yl)carbamate (12.0 g, 30.4 mmol)) as a light brown oil.

LCMS (Method F): m/z 417.1 (M+Na), at 2.28 min.

Step 2: Lithium aluminium hydride (2M in THF, 15.0 mL, 30.0 mmol) was added dropwise at −78° C. to a solution of (9H-fluoren-9-yl)methyl (S)-(3-cyclopropyl-1-(methoxy(methyl)amino)-1-oxopropan-2-yl)carbamate (12.0 g, 30.5 mmol) in dry THF (50 mL) under N₂. After stirring at −78° C. for 2 h under N₂ saturated aqueous NH₄Cl solution (70 mL) was added. The mixture was filtered through celite and the residue was washed with EtOAc (100 mL). The filtrate was concentrated in vacuo to yield crude (9H-fluoren-9-yl)methyl (S)-(1-cyclopropyl-3-oxopropan-2-yl)carbamate (9.20 g) as a light yellow solid that was used for next step without purification.

LCMS (Method G): m/z 336.6 (M+H), at 2.33 and 2.70 min.

Step 3: Benzyl isocyanide (3.56 g, 30.2 mmol) and glacial AcOH (4.71 mL, 82.4 mmol) were added to a solution of (9H-fluoren-9-yl)methyl (S)-(1-cyclopropyl-3-oxopropan-2-yl)carbamate (9.20 g, 27.5 mmol) in DCM (30 mL) at 0° C. After stirring at rt for 2 h 1 N aqueous HCl (20 mL), water (70 mL) and DCM (100 mL) were added. The phases were separated and the aqueous phase was extracted with DCM (2×100 mL). The combined organic layers were washed with saturated aqueous NaHCO₃ solution (100 mL) and dried (Na₂SO₄). After filtering and concentration in vacuo trituration with n-hexane yielded (3S)-3-((((9H-fluoren-9-yl)methoxy)carbonyl)amino)-1-(benzylamino)-4-cyclopropyl-1-oxobutan-2-yl acetate (14.0 g, 2.73 mmol) as a white solid.

LCMS (Method F): m/z 513.0 (M+H), at 2.50 and 2.53 min.

Step 4: Et₃N (8.00 mL, 54.6 mmol) was added to a solution of (3S)-3-((((9H-fluoren-9-yl)methoxy)carbonyl)amino)-1-(benzylamino)-4-cyclopropyl-1-oxobutan-2-yl acetate (14.0 g, 27.3 mmol) in MeOH (200 mL) and the reaction mixture stirred at rt for 2 h. After concentration in vacuo the solid obtained was suspended in water (100 mL) and then filtered. After drying, trituration with n-hexane yielded (9H-fluoren-9-yl)methyl ((2S)-4-(benzylamino)-1-cyclopropyl-3-hydroxy-4-oxobutan-2-yl)carbamate (10.0 g, 21.3 mmol) as a white solid.

LCMS (Method F): m/z 471.3 (M+H), at 2.43 and 2.46 min.

Step 5: (9H-Fluoren-9-yl)methyl((2S)-4-(benzylamino)-1-cyclopropyl-3-hydroxy-4-oxobutan-2-yl)carbamate (10.0 g, 21.3 mmol) was dissolved in 20% piperidine in DMF (100 mL) and stirred at rt for 40 min. After dilution with cold water (500 mL) the resulting suspension was filtered through celite. The filtrate was concentrated in vacuo and triturated with MeCN (70 mL) to yield (3S)-3-amino-N-benzyl-4-cyclopropyl-2-hydroxybutanamide (Intermediate 6, 3.00 g, 7.01 mmol) as a white solid.

LCMS (Method F): m/z 249.3 (M+H), at 0.83 and 0.91 min.

¹H NMR: (400 MHz, DMSO-d₆) δ −0.14-−0.19 (m, 1H), −0.03-−0.06 (m, 1H), 0.28-0.33 (m, 1H), 0.35-0.39 (m, 1H), 0.77-0.81 (q, 1H, J=5.2 Hz), 0.91-0.97 (m, 1H), 1.24-1.31 (m, 1H), 1.53 (br s, 2H), 2.93-2.95 (t, 1H, J=4.4 Hz), 3.83 (s, 1H), 4.21-4.32 (m, 2H), 5.56 (s, 1H), 7.22-7.319 (m, 5H), 8.29-8.32 (t, 1H, J=6.0 Hz).

### Intermediate 7: Methyl 3-amino-4-cyclopropyl-2-hydroxybutanoate hydrochloride

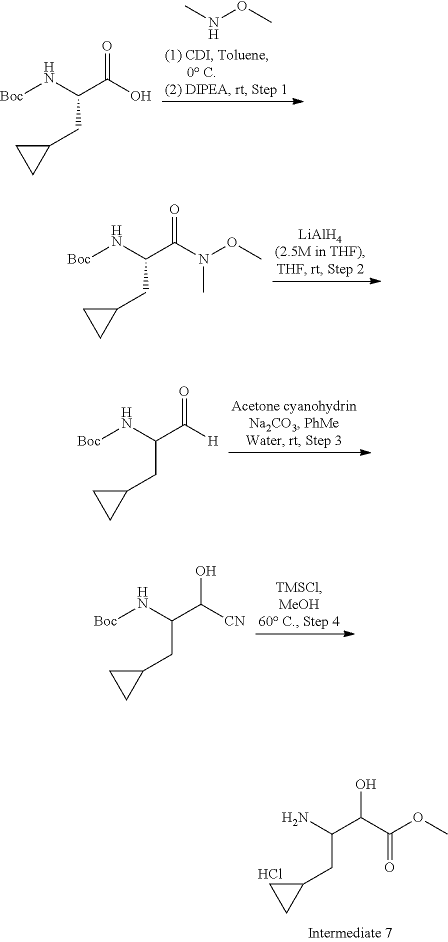

Step 1: (S)-2-((tert-Butoxycarbonyl)amino)-3-cyclopropylpropanoic acid (2.42 g, 10.6 mmol) was dissolved in PhMe (25 mL) at 0° C. under N₂. CDI (1.75 g, 10.8 mmol) was added at 0° C. and the reaction mixture was stirred at 0° C. for 2 h. N,O-Dimethylhydroxylamine hydrochloride (1.34 g, 13.7 mmol) and DIPEA (1.86 mL, 10.8 mmol) were added at 0° C. and the reaction mixture was stirred at rt for 16 h. After partitioning between water (50 mL) and EtOAc (50 mL) the aqueous phase was extracted with EtOAc (2×50 mL). The combined organic phases were dried (Na₂SO₄), filtered and concentrated in vacuo to yield tert-butyl (S)-(3-cyclopropyl-1-(methoxy(methyl)amino)-1-oxopropan-2-yl)carbamate (2.00 g, 7.35 mmol) as an off-white solid.

LCMS (Method F2): m/z 273.1 (M+H), at 2.04 min.

Step 2: LiAlH₄ (2.5M in THF, 2.95 mL, 7.35 mmol) was added to a solution of tert-butyl (S)-(3-cyclopropyl-1-(methoxy(methyl)amino)-1-oxopropan-2-yl)carbamate (2.00 g, 7.35 mmol) in THF (20 mL) at 0° C. under N₂ and the reaction mixture stirred at rt for 1 h. After partitioning between saturated aqueous NH₄Cl solution (50 mL) and EtOAc (60 mL) the aqueous phase was extracted with EtOAc (2×60 mL). The combined organic phases were dried (Na₂SO₄) and concentrated in vacuo to yield tert-butyl (1-cyclopropyl-3-oxopropan-2yl)carbamate (1.30 g, 6.10 mmol) as yellow sticky material that was used without further purification.

TLC: Rf 0.6 (EtOAc/Hexane, 3:7).

Step 3: Sodium carbonate (1.94 g, 18.3 mmol) was added to a solution of tert-butyl (1-cyclopropyl-3-oxopropan-2-yl)carbamate (1.30 g, 6.09 mmol) in PhMe (5 mL) and water (2 mL) and the reaction mixture stirred at rt for 15 min. Acetone cyanohydrin (0.84 mL, 9.14 mmol) was added and the reaction mixture was stirred at rt for 1 h. After dilution with water (20 mL) and acidification with 2N HCl (8 mL) to approximately pH 5 the mixture was extracted with EtOAc (3×60 mL). The combined organic phases were dried (Na₂SO₄) and concentrated in vacuo. Purification by normal phase gradient flash column chromatography on silica, eluting with 0-20% EtOAc in n-hexane yielded tert-butyl (1-cyano-3-cyclopropyl-1-hydroxypropan-2-yl)carbamate (1.00 g, 4.16 mmol) as a brown sticky material.

LCMS (Method F2): m/z 185.2 (M−56), at 1.68 min.

Step 4: Trimethylsilyl chloride (2 mL, 15.7 mmol) was added to a solution of tert-butyl (1-cyano-3-cyclopropyl-1-hydroxypropan-2-yl)carbamate (1.00 g, 4.16 mmol) in MeOH (15 mL) and the mixture was stirred at 60° C. for 3 h. After concentration in vacuo purification by reverse phase gradient flash column chromatography (Silica C18), eluting with 0-12% MeCN in water yielded methyl 3-amino-4-cyclopropyl-2-hydroxybutanoate hydrochloride (0.80 g, 3.81 mmol) as yellow sticky material.

LCMS (Method F2): m/z 174.0 (M+H), at 0.36 min, approximately 69% purity.

### Intermediate 8: 4-Cyclopropyl-2-hydroxy-3-((1 R,2S,5S)-3-(isobutyryl-L-valyl)-6,6-dimethyl-3-azabicyclo[3.1.0]hexane-2-carboxamido)butanoic acid

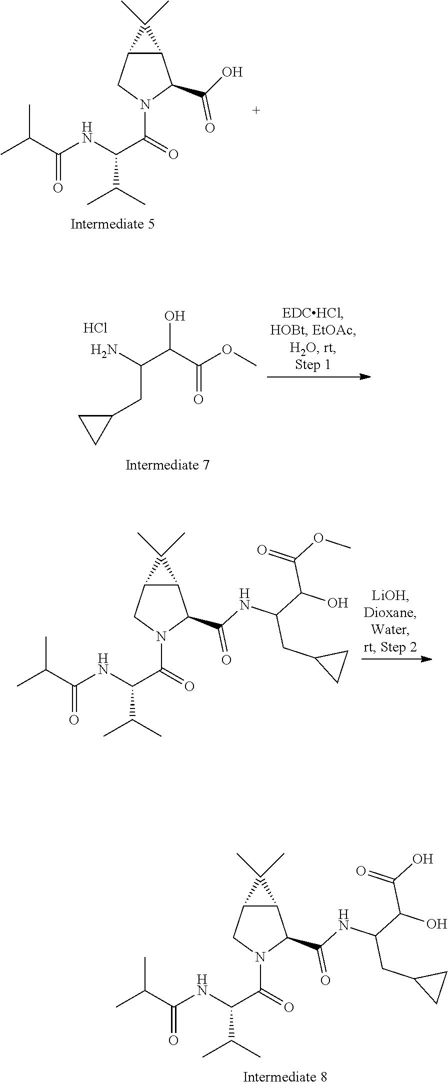

Step 1: EDCl·HCl (0.35 g, 1.85 mmol) and HOBt (0.21 g, 1.54 mmol) were added to a solution of (1 R,2S,5S)-3-(isobutyryl-L-valyl)-6,6-dimethyl-3-azabicyclo[3.1.0]hexane-2-carboxylic acid (Intermediate 5, 0.50 g, 1.54 mmol) and methyl 3-amino-4-cyclopropyl-2-hydroxybutanoate hydrochloride (Intermediate 7, 0.35 g, 1.70 mmol) in EtOAc (5 mL) and water (0.5 mL). After stirring at rt for 16 h the reaction mixture was partitioned between water (30 mL) and EtOAc (50 mL). The phases were separated and the aqueous phase extracted with EtOAc (2×50 mL). The combined organic phases were dried (Na₂SO₄), filtered and concentrated in vacuo. Purification by reverse phase gradient flash column chromatography (Silica C18), eluting with 0-40% MeCN in water yielded methyl 4-cyclopropyl-2-hydroxy-3-((1 R,2S,5S)-3-(isobutyryl-L-valyl)-6,6-dimethyl-3-azabicyclo[3.1.0]hexane-2-carboxamido)butanoate (0.40 g, 0.83 mmol) as an off-white solid.

LCMS (Method F): m/z 480.3 (M+H), at 1.56 min.

Step 2: LiOH monohydrate (89 mg, 2.18 mmol) was added to a solution of methyl 4-cyclopropyl-2-hydroxy-3-((1 R,2S,5S)-3-(isobutyryl-L-valyl)-6,6-dimethyl-3-azabicyclo[3.1.0]hexane-2-carboxamido)butanoate (0.21 g, 0.43 mmol) in 1,4-dioxane (1 mL) and water (1 mL). After stirring at rt for 3 h, water (50 mL) was added, and the reaction mixture was extracted with EtOAc (2×30 mL). The aqueous phase was acidified with 1 N HCl (1.5 mL) to approximately pH 2 and extracted with EtOAc (3×30 mL). The combined organic phases were dried (Na₂SO₄) and concentrated in vacuo to yield 4-cyclopropyl-2-hydroxy-3-((1 R,2S,5S)-3-(isobutyryl-L-valyl)-6,6-dimethyl-3-azabicyclo[3.1.0]hexane-2-carboxamido)butanoic acid (0.18 g, 0.39 mmol) as brown sticky material.

LCMS (Method F2): m/z 466.1 (M+H), at 1.69, 1.77 and 1.83 min.

### Intermediate 9: tert-Butyl N-((1 S)-1-((1 R,2S,5S)-2-((3-amino-1-(cyclopropylmethyl)-2-hydroxy-3-oxo-propyl)carbamoyl)-6,6-dimethyl-3-azabicyclo[3.1.0]hexane-3-carbonyl)-2-methyl-2-phenyl-propyl)carbamate

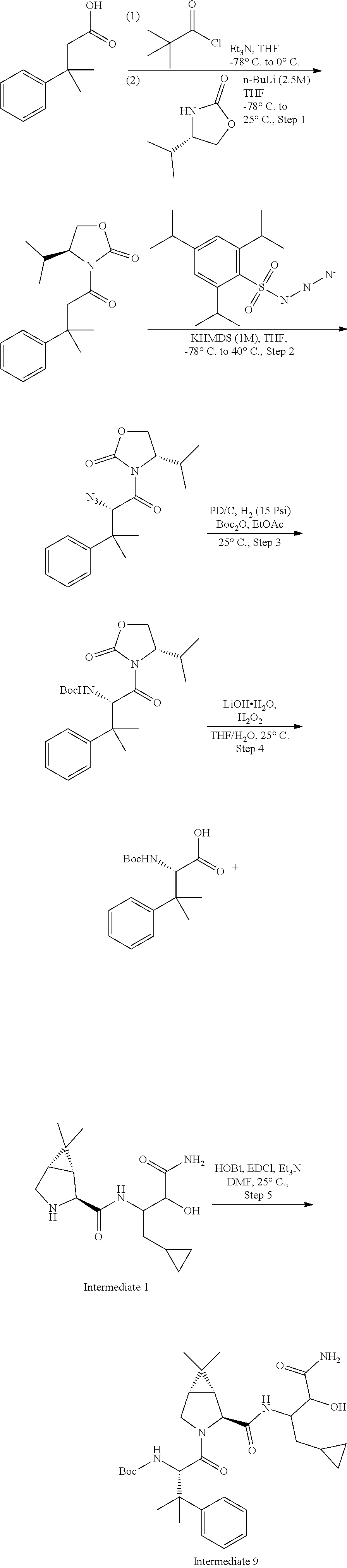

Step 1: To a mixture of 3-methyl-3-phenyl-butanoic acid (4.50 g, 25.3 mmol) in THF (60 mL) was added Et₃N (3.83 g, 37.9 mmol, 5.27 mL) and 2,2-dimethylpropanoyl chloride (3.35 g, 27.8 mmol, 3.42 mL) dropwise at −78° C. under N₂. The reaction mixture was stirred at −78° C. until a white solid was formed. The reaction mixture was warmed to 0° C. and stirred for 1 h before cooling to −78° C. (mixture A). To a solution of (4S)-4-isopropyloxazolidin-2-one (6.52 g, 50.5 mmol) in THF (80 mL) was added n-BuLi (2.5 M in n-hexane, 20.2 mL) dropwise at −78° C. under N₂ (mixture B). Mixture B was added to Mixture A at −78° C. and the reaction mixture was stirred at −78° C. for 2 h, then 25° C. for 12 h. The reaction mixture was slowly quenched with saturated aqueous NH₄Cl solution (100 mL) at 0° C. and then stirred at 25° C. for 1 h. The mixture was extracted with ethyl acetate (50 mL×3). The combined organic phases were washed with brine (100 mL), dried with anhydrous Na₂SO₄, filtered and concentrated in vacuo. The residue was purified by gradient flash column chromatography on silica gel eluting with petroleum ether to petroleum ether/ethyl acetate 5/1, followed by preparative reverse phase HPLC (formic acid as additive) to yield (4S)-4-isopropyl-3-(3-methyl-3-phenyl-butanoyl)oxazolidin-2-one (6.15 g, 21.3 mmol) as a yellow oil.

LCMS (Method A): m/z 290.2 (M+H), at 0.91 min.

1H NMR: (400 MHz, CDCl₃) δ 0.74 (3H, d, J=6.8 Hz), 0.81 (3H, d, J=6.8 Hz), 1.50 (6H, d, J=1.6 Hz), 2.12-2.20 (1H, m), 3.36 (2H, d, J=4.0 Hz), 4.01-4.09 (2H, m), 4.20-4.24 (1H, m), 7.15-7.21 (1H, m), 7.27-7.33 (2H, m), 7.37-7.42 (2H, m).

Preparative reverse phase HPLC (formic acid) method for the purification of Intermediate 9, Step 1. Instrument: Shimadzu LC-20AP; Column: Phenomenex Luna C18 250×80 mm×15 μm; Mobile phase: A=0.225% formic acid in water (v/v), B=MeCN; Gradient: 55-85% B in A over 35 min; Flow rate: 140 mL/min; Column temperature: 40° C.; Wavelength: 220 nm, 254 nm.

Step 2: To a mixture of (4S)-4-isopropyl-3-(3-methyl-3-phenyl-butanoyl)oxazolidin-2-one (6.00 g, 20.7 mmol) in THF (50 mL) was added KHMDS (1M in THF, 22.8 mL) dropwise at −78° C. under N₂. The reaction mixture was stirred at −78° C. for 1 h. A solution of N-diazo-2,4,6-triisopropyl-benzenesulfonamide (8.02 g, 25.9 mmol) in THF (30 mL) was then added dropwise at −78° C. The reaction mixture was stirred at −78° C. for 0.5 h before the addition of HOAc (5.73 g, 95.4 mmol, 5.46 mL) at −78° C. The reaction mixture was stirred at 40° C. for 2 h before quenching with saturated aqueous NH₄Cl solution (100 mL) at 0° C. and extraction with ethyl acetate (50 mL×3). The combined organic phases were washed with brine (50 mL), saturated aqueous NaHCO₃ solution (50 mL), dried with anhydrous Na₂SO₄, filtered and concentrated in vacuo. Purification by gradient flash column chromatography on silica gel eluting with petroleum ether to petroleum ether/ethyl acetate 20/1 yielded (4S)-3-[(2S)-2-azido-3-methyl-3-phenyl-butanoyl]-4-isopropyl-oxazolidin-2-one (5.92 g, 17.9 mmol) as a light yellow oil.

LCMS (Method A): m/z 303.2 (M+H-28)⁺, at 0.94 min.

¹H NMR: (400 MHz, CDCl₃) δ 0.83 (6H, dd, J=15.6, 7.2 Hz), 1.54-1.56 (6H, m), 2.28-2.34 (1H, m), 3.57 (1H, t, J=8.4 Hz), 3.87-3.91 (1H, m), 3.97 (1H, dd, J=8.8, 2.0 Hz), 5.66 (1H, s), 7.25-7.28 (1H, m), 7.30-7.36 (2H, m), 7.38-7.43 (2H, m).

Step 3: A mixture of (4S)-3-[(2S)-2-azido-3-methyl-3-phenyl-butanoyl]-4-isopropyl-oxazolidin-2-one (5.70 g, 17.3 mmol), Boc₂O (8.28 g, 38.0 mmol, 8.72 mL) and Pd/C (500 mg, 10% purity, w/w) in EtOAc (50 mL) was stirred at 25° C. for 12 h under H₂ (15 PSI). The reaction mixture was filtered and the filtrate was concentrated in vacuo. The resulting residue was purified by gradient flash column chromatography on silica gel eluting with petroleum ether to petroleum ether/ethyl acetate 10/1, followed by preparative HPLC (formic acid as additive) and then normal phase preparative HPLC (NH₃—H₂O as additive) to yield tert-butyl N-[(1S)-1-[(4S)-4-isopropyl-2-oxo-oxazolidine-3-carbonyl]-2-methyl-2-phenyl-propyl]carbamate (6.50 g, 16.1 mmol) as a light yellow oil.

LCMS (Method A): m/z 405.2 (M+H), at 0.95 min.

¹H NMR: (400 MHz, CDCl₃) δ 0.76-0.83 (6H, m), 1.41 (3H, s), 1.43 (9H, s), 1.48 (3H, s), 2.21-2.29 (1H, m), 3.45 (1H, t, J=8.4 Hz), 3.79-3.83 (1H, m), 3.90 (1H, dd, J=8.8, 2.0 Hz), 4.86-5.32 (1H, m), 6.13 (1H, d, J=10.0 Hz), 7.20-7.26 (1H, m), 7.28-7.34 (2H, m), 7.41 (2H, d, J=7.6 Hz).

Preparative reverse phase HPLC (formic acid) method for the purification of Intermediate 9, Step 3. Instrument: Shimadzu LC-20AP; Column: Phenomenex Luna C18 250×80 mm×10 μm; Mobile phase: A=0.225% formic acid in water (v/v), B=MeCN; Gradient: 48-78% B in A over 20 min; Flow rate: 100 mL/min; Column temperature: 40° C.; Wavelength: 220 nm, 254 nm.

Preparative normal phase HPLC (NH₃·H₂O) method for the purification of Intermediate 9, Step 3. Instrument: Shimadzu LC-20AP; Column: Welch Ultimate XB-SiOH 250×50 mm×10 μm; Mobile phase: A=Hexane, B=0.1% NH₃—H₂O in EtOH (v/v), 2% B in A over 15 min; Flow rate: 100 mL/min; Column temperature: 40° C.; Wavelength: 220 nm, 254 nm.

Step 4: To a mixture of tert-butyl N-[(1 S)-1-[(4S)-4-isopropyl-2-oxo-oxazolidine-3-carbonyl]-2-methyl-2-phenyl-propyl]carbamate (3.00 g, 7.42 mmol) and 1 N aqueous LiOH solution (22.3 mL) in THF (80 mL) and H₂O (20 mL) was added dropwise 30% aqueous H₂O₂ solution (10.1 g, 89.0 mmol, 8.60 mL) at 0° C. under N₂. The reaction mixture was stirred at 25° C. for 12 h before the addition of H₂O (100 mL) and saturated aqueous Na₂SO₃ solution (300 mL) at 0° C. The mixture was stirred at 25° C. for 1 h then the pH was adjusted to approximately 7 by addition of 1 N aqueous HCl and the mixture was extracted with CH₂Cl₂ (50 mL×3). The combined organic phases were washed with brine (100 mL) and saturated aqueous Na₂SO₃ solution (100 mL), dried with anhydrous Na₂SO₄, filtered and concentrated in vacuo. Purification by gradient flash column chromatography on silica gel eluting with petroleum ether/ethyl acetate (10/1 to 1/1), followed by preparative HPLC (formic acid as additive) and preparative HPLC (NH₄HCO₃ as additive) yielded (2S)-2-(tert-butoxycarbonylamino)-3-methyl-3-phenyl-butanoic acid (1.53 g, 5.22 mmol) as a light yellow solid.

LCMS (Method A2): m/z 194.1 (M+H-100)⁺, at 2.87 min.

¹H NMR: (400 MHz, DMSO-d₆) δ 1.28 (9H, s), 1.30 (6H, d, J=5.6 Hz), 4.01 (1H, d, J=8.8 Hz), 5.86 (1H, d, J=9.2 Hz), 7.07-7.13 (1H, m), 7.22 (2H, t, J=7.6 Hz), 7.32 (2H, d, J=7.6 Hz).

Preparative reverse phase HPLC (formic acid) method for the purification of Intermediate 9, Step 4. Instrument: Shimadzu LC-20AP; Column: Phenomenex Luna C18 250×70 mm×10 μm; Mobile phase: A=0.225% formic acid in water (v/v), B=MeCN; Gradient: 30-60% B in A over 20 min; Flow rate: 140 mL/min; Column temperature: 40° C.; Wavelength: 220 nm, 254 nm.

Preparative reverse phase HPLC (NH₄HCO₃) method for the purification of Intermediate 9, Step 4. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 14-44% B in A over 10 min; Flow rate: 25 mL/min; Column temperature: 40° C.; Wavelength: 220 nm, 254 nm.

Step 5: A mixture of (2S)-2-(tert-butoxycarbonylamino)-3-methyl-3-phenyl-butanoic acid (Intermediate 9, Step 4 product, 669 mg, 2.28 mmol), HOBt (308 mg, 2.28 mmol) and EDCI (437 mg, 2.28 mmol) in DMF (5 mL) was stirred at 25° C. for 0.2 h. (1 R,2S,5S)—N-[3-Amino-1-(cyclopropylmethyl)-2-hydroxy-3-oxo-propyl]-6,6-dimethyl-3-azabicyclo[3.1.0]hexane-2-carboxamide (Intermediate 1, 450 mg, 1.52 mmol) and Et₃N (308 mg, 3.04 mmol, 0.42 mL) were added and the reaction mixture was stirred at 25° C. for 2 h. The reaction mixture was diluted with H₂O (50 mL) and the mixture was extracted with EtOAc (30 mL×3). The combined organic phases were washed with brine (30 mL), dried with anhydrous Na₂SO₄, filtered and concentrated in vacuo. The residue was purified by preparative HPLC (formic acid as additive) followed by preparative HPLC (NH₄HCO₃ as additive) to yield tert-butyl N-((1 S)-1-((1 R,2S,5S)-2-((3-amino-1-(cyclopropylmethyl)-2-hydroxy-3-oxo-propyl)carbamoyl)-6,6-dimethyl-3-azabicyclo[3.1.0]hexane-3-carbonyl)-2-methyl-2-phenyl-propyl)carbamate (Intermediate 9, 42.0 mg, 0.07 mmol) as a light yellow solid.

LCMS (Method A): m/z 571.2 (M+H)⁺, at 0.90 min.

¹H NMR: (400 MHz, CDCl₃) δ 0.06-0.22 (2H, m), 0.40-0.58 (2H, m), 0.66-0.76 (1H, m), 0.76-0.89 (3H, m), 0.96-0.99 (3H, m), 1.22-1.37 (2H, m), 1.38-1.43 (15H, m), 1.44-1.48 (2H, m), 3.69-3.86 (1H, m), 4.20-4.33 (2H, m), 4.61-4.76 (1H, m), 4.92-5.28 (1H, m), 5.46-5.71 (1H, m), 7.29-7.43 (5H, m).

Preparative reverse phase HPLC (formic acid) method for the purification of Intermediate 9, Step 5. Instrument: Shimadzu LC-20AP; Column: Phenomenex Luna C18 75×30 mm×3 μm; Mobile phase: A=0.225% formic acid in water (v/v), B=MeCN; Gradient: 45-75% B in A over 7 min; Flow rate: 25 mL/min; Column temperature: 40° C.; Wavelength: 220 nm, 254 nm.

Preparative reverse phase HPLC (NH₄HCO₃) method for the purification of Intermediate 9, Step 5. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 32-62% B in A over 10 min; Flow rate: 25 mL/min; Column temperature: 40° C.; Wavelength: 220 nm, 254 nm.

### Intermediate 10: 4-Cyclopropyl-2-hydroxy-3-((1 R,2S,5S)-3-((S)-2-isobutyramido-3,3-dimethylbutanoyl)-6,6-dimethyl-3-azabicyclo[3.1.0]hexane-2-carboxamido)butanoic acid

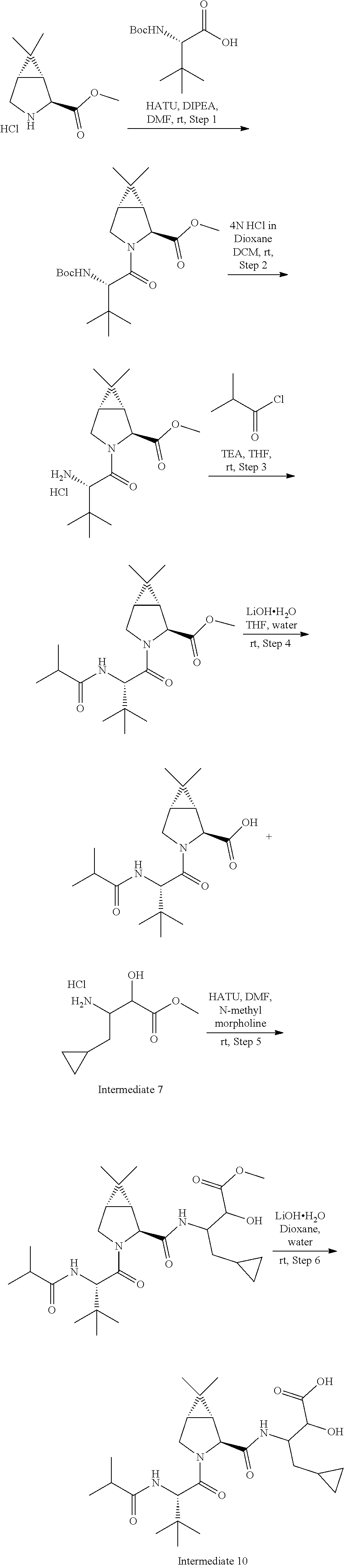

Step 1: (S)-2-((tert-Butoxycarbonyl)amino)-3,3-dimethylbutanoic acid (5.00 g, 21.6 mmol) and methyl (1 R,2S,5S)-6,6-dimethyl-3-azabicyclo[3.1.0]hexane-2-carboxylate hydrochloride (5.34 g, 26.0 mmol) were dissolved in DMF (30 mL) at rt. HATU (9.89 g, 26.0 mmol) was added and the reaction mixture was stirred at rt for 30 min before the addition of DIPEA (11.3 mL, 64.9 mmol) and the reaction mixture was stirred at rt for 3 h. Cold water (100 mL) was added and the resulting precipitate was collected by filtration to yield crude methyl (1 R,2S,5S)-3-((S)-2-((tert-butoxycarbonyl)amino)-3,3-dimethylbutanoyl)-6,6-dimethyl-3-azabicyclo[3.1.0]hexane-2-carboxylate (9.30 g) as a brown sticky solid.

LCMS (Method F3): m/z 327.4 (M−56), at 2.35 and 2.37 min.

Step 2: 4N HCl in 1,4-dioxane (90 mL) was added to a solution of methyl (1 R,2S,5S)-3-((S)-2-((tert-butoxycarbonyl)amino)-3,3-dimethylbutanoyl)-6,6-dimethyl-3-azabicyclo[3.1.0]hexane-2-carboxylate (9.30 g) in DCM (30 mL) under N₂. After stirring at rt for 2 h the reaction mixture was concentrated in vacuo and purified by trituration with diethyl ether (3×10 mL) to yield methyl (1R,2S,5S)-3-((S)-2-amino-3,3-dimethylbutanoyl)-6,6-dimethyl-3-azabicyclo[3.1.0]hexane-2-carboxylate hydrochloride (6.70 g, 21.0 mmol) as white solid.

LCMS (Method F3): m/z 283.5 (M+H), at 1.14 and 1.28 min.

Step 3: Et₃N (8.80 mL, 63.2 mmol) was added drop wise at 0° C. to a solution of methyl (1 R,2S,5S)-3-((S)-2-amino-3,3-dimethylbutanoyl)-6,6-dimethyl-3-azabicyclo[3.1.0]hexane-2-carboxylate hydrochloride (6.70 g, 21.0 mmol) in THF (40 mL) and the reaction mixture was stirred for 15 min. Isobutyryl chloride (2.23 g, 21.1 mmol) was added and the reaction mixture was stirred at rt for 30 min before the addition of water (200 mL) and extraction with EtOAc (3×100 mL). The combined organic phases were dried (Na₂SO₄), concentrated in vacuo and purification by reverse phase gradient flash column chromatography (Silica C18), eluting with 0% to 42% MeCN in water yielded methyl (1 R,2S,5S)-3-((S)-2-isobutyramido-3,3-dimethylbutanoyl)-6,6-dimethyl-3-azabicyclo[3.1.0]hexane-2-carboxylate (4.90 g, 13.9 mmol) as a brown sticky solid.

LCMS (Method F3): m/z 353.5 (M+H), at 1.88 and 1.96 min.

Step 4: LiOH monohydrate (2.92 g, 69.6 mmol) was added to a solution of methyl (1 R,2S,5S)-3-((S)-2-isobutyramido-3,3-dimethylbutanoyl)-6,6-dimethyl-3-azabicyclo[3.1.0]hexane-2-carboxylate (4.90 g, 13.9 mmol) in 1,4-dioxane (20 mL) and water (15 mL) and the reaction mixture was stirred at rt for 4 h before the addition of water (200 mL) and EtOAc (100 mL). The phases were separated, the aqueous phase was acidified with 1N HCl (15 mL) to approximately pH 2 and extracted with EtOAc (2×100 mL). The combined organic phases were dried (Na₂SO₄) and concentrated in vacuo to yield crude (1 R,2S,5S)-3-((S)-2-isobutyramido-3,3-dimethylbutanoyl)-6,6-dimethyl-3-azabicyclo[3.1.0]hexane-2-carboxylic acid (4.80 g) as a white solid.

LCMS (Method F3): m/z 339.5 (M+H), at 1.59 and 1.74 min.

Step 5: HATU (3.37 g, 8.86 mmol) was added to a solution of (1 R,2S,5S)-3-((S)-2-isobutyramido-3,3-dimethylbutanoyl)-6,6-dimethyl-3-azabicyclo[3.1.0]hexane-2-carboxylic acid (1.50 g, 4.43 mmol) and methyl 3-amino-4-cyclopropyl-2-hydroxybutanoate hydrochloride (1.11 g, 5.32 mmol) in DMF (15 mL) and the reaction mixture was stirred for 15 min at rt. N-Methyl morpholine (1.95 mL, 17.7 mmol) was added and the reaction mixture was stirred at rt for 30 min before concentration in vacuo. Purification by reverse phase gradient flash column chromatography (Silica C18), eluting with 0% to 46% MeCN in water yielded methyl 4-cyclopropyl-2-hydroxy-3-((1 R,2S,5S)-3-((S)-2-isobutyramido-3,3-dimethylbutanoyl)-6,6-dimethyl-3-azabicyclo[3.1.0]hexane-2-carboxamido)butanoate (1.70 g, 3.44 mmol) as a white solid.

LCMS (Method F3): m/z 494.5 (M+H), at 1.69 and 1.75 min.

Step 6: LiOH monohydrate (0.11 g, 2.53 mmol) was added to a solution of methyl 4-cyclopropyl-2-hydroxy-3-((1 R,2S,5S)-3-((S)-2-isobutyramido-3,3-dimethylbutanoyl)-6,6-dimethyl-3-azabicyclo[3.1.0]hexane-2-carboxamido)butanoate (0.25 g, 0.51 mmol) in 1,4-dioxane (2 mL) and water (2 mL) and the reaction mixture was stirred at rt for 2 h. Water (70 mL) and EtOAc (50 mL) were added and the aqueous layer was acidified with 1 N HCl (3 mL) to approximately pH 2 and extracted with EtOAc (2×50 mL). The combined organic phases were dried (Na₂SO₄) and concentrated in vacuo to yield 4-cyclopropyl-2-hydroxy-3-((1 R,2S,5S)-3-((S)-2-isobutyramido-3,3-dimethylbutanoyl)-6,6-dimethyl-3-azabicyclo[3.1.0]hexane-2-carboxamido)butanoic acid (Intermediate 10, 0.20 g, 0.39 mmol) as a white solid.

LCMS (Method F3): m/z 480.5 (M+H), at 1.55 and 1.58 min.

### SYNTHESIS OF EXAMPLES

### Example 1: (1 R,2S,5S)—N-(4-Amino-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide

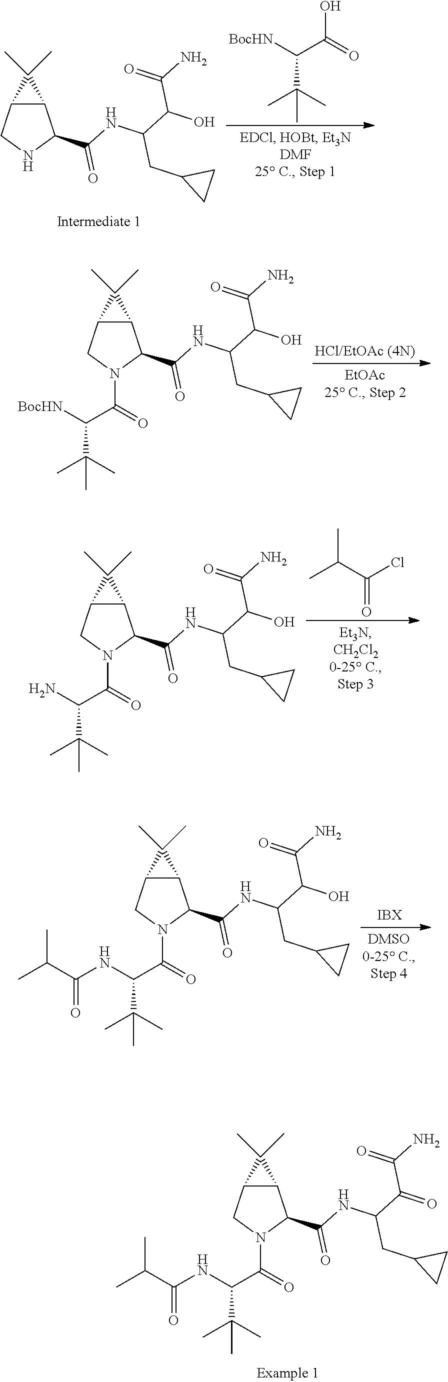

Step 1: To a mixture of (2S)-2-(tert-butoxycarbonylamino)-3,3-dimethyl-butanoic acid (305 mg, 1.32 mmol) in DMF (5 mL) was slowly added HOBt (178 mg, 1.32 mmol) and EDCl·HCl (253 mg, 1.32 mmol) at 25° C. and the mixture stirred for approximately 12 min, then (1 R,2S,5S)—N-[3-amino-1-(cyclopropylmethyl)-2-hydroxy-3-oxo-propyl]-6,6-dimethyl-3-azabicyclo[3.1.0]hexane-2-carboxamide (Intermediate 1, 300 mg, 1.02 mmol) and Et₃N (206 mg, 2.03 mmol, 0.28 mL) were added. The reaction mixture was stirred at 25° C. for 2 h under N₂. H₂O (50 mL) was added, and the mixture was extracted with EtOAc (20 mL×3). The combined organic phases were washed with brine (30 mL), dried with anhydrous Na₂SO₄, filtered and concentrated in vacuo. The residue was purified by preparative HPLC (formic acid as additive) to yield tert-butyl N-[(1 S)-1-[(1 R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carbonyl]-2,2-dimethyl-propyl]carbamate (251 mg, 0.49 mmol) as a white solid.

LCMS (Method A): m/z 509.2 (M+H), at 1.08 min.

¹H NMR: (400 MHz, CDCl₃) δ 0.05-0.27 (2H, m), 0.41-0.53 (2H, m), 0.68-0.81 (1H, m), 0.86-0.88 (3H, m), 0.97 (3H, d, J=3.6 Hz), 0.98-1.06 (9H, m), 1.39 (9H, s), 1.43 (2H, br d, J=6.8 Hz), 1.52-1.57 (1H, m), 1.66-1.89 (1H, m), 3.78-4.19 (3H, m), 4.19-4.34 (2H, m), 4.34-4.48 (1H, m), 6.78-7.20 (1H, m).

Preparative HPLC (formic acid) method for the purification of Step 1. Instrument: Shimadzu LC-20AP; Column: Phenomenex luna C18 150×40 mm×15 μm; Mobile phase: A=0.225% formic acid in water (v/v), B=MeCN; Gradient: 40-70% B in A over 10 min; Flow rate: 60 mL/min; Column temperature: 40° C.; Wavelength: 220 nm, 254 nm.

Step 2: HCl in EtOAc (4N, 5 mL) was added to a mixture of tert-butyl N-[(1 S)-1-[(1 R,2S,5S)-2-[[3-amino-1-(cyclopropylmethyl)-2-hydroxy-3-oxo-propyl]carbamoyl]-6,6-dimethyl-3-azabicyclo[3.1.0]hexane-3-carbonyl]-2,2-dimethyl-propyl]carbamate (220 mg, 0.43 mmol) in EtOAc (5 mL) at 25° C., and the resulting mixture was stirred at 25° C. for 1 h under N₂. The reaction mixture was concentrated in vacuo to yield (1 R,2S,5S)—N-[3-amino-1-(cyclopropylmethyl)-2-hydroxy-3-oxo-propyl]-3-[(2S)-2-amino-3,3-dimethyl-butanoyl]-6,6-dimethyl-3-azabicyclo[3.1.0]hexane-2-carboxamide (185 mg) as a white solid that was used crude without purification.

LCMS (Method A): m/z 409.2 (M+H), at 0.68 min.

Step 3: To a mixture of (1R,2S,5S)—N-[3-amino-1-(cyclopropylmethyl)-2-hydroxy-3-oxo-propyl]-3-[(2S)-2-amino-3,3-dimethylbutanoyl]-6,6-dimethyl-3-azabicyclo[3.1.0]hexane-2-carboxamide (180 mg, crude) and Et₃N (0.12 mL, 0.88 mmol,) in DCM (5 mL) was added 2-methylpropanoyl chloride (0.06 mL, 0.57 mmol) at 0° C., the resulting mixture was stirred at 25° C. for 1 h under N₂. The reaction mixture was concentrated in vacuo and purified by preparative HPLC (formic acid as additive) to afford desired Step 3 product (1 R,2S,5S)—N-[3-amino-1-(cyclopropylmethyl)-2-hydroxy-3-oxo-propyl]-3-[(2S)-3,3-dimethyl-2-(2-methylpropanoylamino)butanoyl]-6,6-dimethyl-3-azabicyclo[3.1.0]hexane-2-carboxamide (121 mg, 0.25 mmol) as a white solid.

LCMS (Method A): m/z 479.2 (M+H), at 0.77 min.

¹H NMR: (400 MHz, CDCl₃) δ 0.05-0.26 (2H, m), 0.39-0.53 (2H, m), 0.68-0.81 (1H, m), 0.81-0.88 (3H, m), 0.92-0.99 (6H, m), 0.99-1.07 (9H, m), 1.13 (3H, d, J=7.2 Hz), 1.17-1.39 (1H, m), 1.41-1.60 (2H, m), 1.70-1.82 (1H, m), 2.32-2.44 (1H, m), 3.81-4.21 (3H, m), 4.25-4.32 (1H, m), 4.38-4.52 (1H, m), 4.54-4.66 (1H, m), 6.10-6.27 (1H, m), 6.76-7.00 (1H, m).

Preparative HPLC (formic acid) method for the purification of Step 3. Instrument: Shimadzu LC-20AP; Column: Unisil 3-100 C18 Ultra 150×25 mm×3 μm; Mobile phase: A=0.225% formic acid in water (v/v), B=MeCN; Gradient: 30-60% B in A over 10 min; Flow rate: 25 mL/min; Column temperature: 40° C.; Wavelength: 220 nm, 254 nm.

Step 4: To a mixture of (1R,2S,5S)—N-[3-amino-1-(cyclopropylmethyl)-2-hydroxy-3-oxo-propyl]-3-[(2S)-3,3-dimethyl-2-(2-methylpropanoylamino)butanoyl]-6,6-dimethyl-3-azabicyclo[3.1.0]hexane-2-carboxamide (110 mg, 0.23 mmol) in DMSO (3 mL) was added 2-iodylbenzoic acid (161 mg, 0.57 mmol) at 0° C., and the resulting mixture was stirred at 25° C. for 12 h under N₂. H₂O (0.5 mL) was added and the resulting mixture was filtered and concentrated in vacuo. The residue was purified by preparative HPLC (NH₄HCO₃ as additive) to yield Example 1, (1 R,2S,5S)—N-[3-amino-1-(cyclopropylmethyl)-2,3-dioxo-propyl]-3-[(2S)-3,3-dimethyl-2-(2-methylpropanoylamino)butanoyl]-6,6-dimethyl-3-azabicyclo[3.1.0]hexane-2-carboxamide (55.5 mg, 0.12 mmol) as a white solid.

LCMS (Method D): m/z 477.3 (M+H), at 2.69 min.

¹H NMR: (400 MHz, CDCl₃) δ 0.00-0.16 (2H, m), 0.39-0.50 (2H, m), 0.69-0.76 (1H, m), 0.81 (2H, d, J=2.4 Hz), 0.95-1.01 (9H, m), 1.03 (2H, s), 1.08 (3H, d, J=6.8 Hz), 1.14 (3H, d, J=6.8 Hz), 1.46-1.81 (6H, m), 2.33-2.40 (1H, m), 3.76-3.86 (1H, m), 4.01 (1H, br d, J=10.8 Hz), 4.46 (1H, d, J=13.6 Hz), 4.60-4.69 (1H, m), 5.35-5.40 (1H, m), 5.74 (1H, br s), 6.08-6.16 (1H, m), 6.79 (1H, br d, J=10.8 Hz), 7.27-7.53 (1H, m).

Preparative HPLC (NH₄HCO₃) method for the purification of Example 1. Instrument: Shimadzu LC-20AP; Column: Waters Xbridge 150×25 mm×5 μm; Mobile phase: A=10 mM NH₄HCO₃ aqueous solution (v/v), B=MeCN; Gradient: 25-55% B in A over 10 min; Flow rate: 25 mL/min; Column temperature: 40° C.; Wavelength: 220 nm, 254 nm.

### Example 2: (1 R,2S,5S)—N-[3-Amino-1-(cyclopropylmethyl)-2,3-dioxo-propyl]-3-[(2S)-2-(cyclopropanecarbonylamino)-3-methylbutanoyl]-6,6-dimethyl-3-azabicyclo[3.1.0]hexane-2-carboxamide

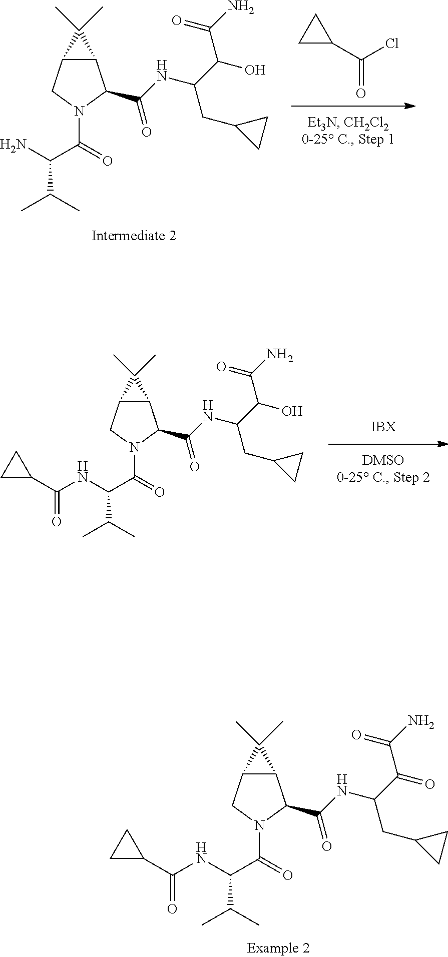

Steps 1 and 2: The title compound (34.3 mg, 0.07 mmol, white solid) was prepared from Intermediate 2 (125 mg, 0.32 mmol) and cyclopropanecarbonyl chloride (39.8 mg, 0.38 mmol, 0.03 mL) using similar procedures to those detailed for Example 1 (Steps 3 and 4). Example 2 was purified by preparative HPLC (NH₄HCO₃ as additive).

LCMS (Method D): m/z 461.3 (M+H), at 2.41 min.

¹H NMR: (400 MHz, CDCl₃) δ 0.03-0.13 (2H, m), 0.45-0.49 (2H, m), 0.67-0.77 (4H, m), 0.86 (3H, s), 0.93-0.96 (6H, m), 1.03 (3H, s), 1.05 (1H, br d, J=3.6 Hz), 1.37-1.41 (1H, m), 1.48-1.51 (1H, m), 1.62-1.68 (1H, m), 1.75-1.83 (2H, m), 2.00-2.06 (1H, m), 3.77-3.84 (1H, m), 3.97 (1H, br d, J=10.4 Hz), 4.44-4.55 (2H, m), 5.26-5.43 (1H, m), 5.69-5.91 (1H, m), 6.33-6.44 (1H, m), 6.75-6.85 (1H, m), 7.26-7.49 (1H, m).

Preparative HPLC (NH₄HCO₃) method for the purification of Example 2. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 15-45% B in A over 10 min; Flow rate: 25 mL/min; Column temperature: 40° C.; Wavelength: 220 nm, 254 nm.

### Example 3: (1R,2S,5S)—N-(4-Amino-1-cyclopropyl-3,4-dioxobutan-2-yl)-3-(isobutyryl-L-alloisoleucyl)-6,6-dimethyl-3-azabicyclo[3.1.0]hexane-2-carboxamide

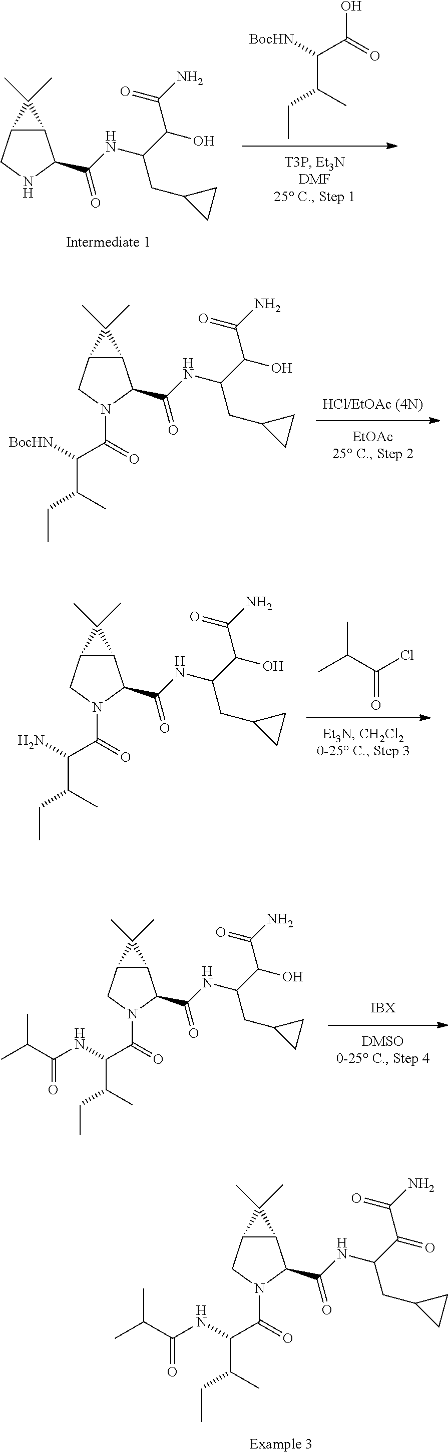

The title compound (50.0 mg, 0.10 mmol, white solid) was prepared from Intermediate 1 (380 mg, 1.29 mmol) and (2S,3R)-2-(tert-butoxycarbonylamino)-3-methyl-pentanoic acid (372 mg, 1.61 mmol) using similar procedures to those detailed for Intermediate 1 (Step 90) and Example 1 (Steps 2-4). Example 3 was purified by preparative HPLC (NH₄HCO₃ as additive).

LCMS (Method D): m/z 477.3 (M+H), at 2.59 min.

1H NMR: (400 MHz, CDCl₃) δ 0.00-0.16 (2H, m), 0.41-0.52 (2H, m), 0.66-0.77 (1H, m), 0.84 (3H, s), 0.85-0.88 (3H, m), 0.89-0.96 (3H, m), 1.03 (3H, s), 1.06-1.10 (3H, m), 1.13 (3H, m), 1.45-1.56 (2H, m), 1.63-1.70 (1H, m), 1.71-1.90 (4H, m), 2.30-2.42 (1H, m), 3.77-3.84 (1H, m), 4.04 (1H, d, J=10.8 Hz), 4.44 (1H, d, J=9.6 Hz), 4.51-4.58 (1H, m), 5.35-5.40 (1H, m), 5.96 (1H, br s), 6.15 (1H, br t, J=9.8 Hz), 6.80 (1H, br d, J=11.2 Hz), 7.27-7.47 (1H, m).

Preparative HPLC (NH₄HCO₃) method for the purification of Example 3. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 22-52% B in A over 10 min; Flow rate: 25 mL/min; Column temperature: 40° C.; Wavelength: 220 nm, 254 nm.

### Example 4: (1 R,2S,5S)-3-(Acryloyl-L-valyl)-N-(4-amino-1-cyclopropyl-3,4-dioxobutan-2-yl)-6,6-dimethyl-3-azabicyclo[3.1.0]hexane-2-carboxamide

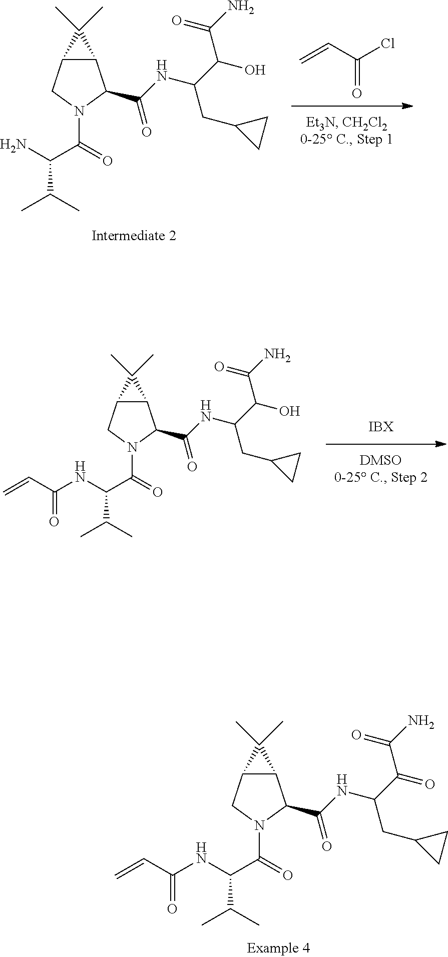

The title compound (32.4 mg, 0.07 mmol, white solid) was prepared from Intermediate 2 (130 mg, 0.33 mmol) and prop-2-enoyl chloride (38.8 mg, 0.43 mmol, 0.03 mL) using similar procedures to those detailed for Example 1 (Steps 3 and 4). Example 4 was purified twice by preparative HPLC (NH₄HCO₃ as additive, followed by formic acid as additive).

LCMS (Method D): m/z 447.0 (M+H), at 2.21 min.

¹H NMR: (400 MHz, CDCl₃) δ 0.00-0.14 (2H, m), 0.40-0.51 (2H, m), 0.68-0.74 (1H, m), 0.84-0.89 (3H, m), 0.89-0.93 (3H, m), 0.95-1.00 (3H, m), 1.02-1.07 (3H, m), 1.48-1.56 (1H, m), 1.61-1.67 (1H, m), 1.73-1.81 (1H, m), 1.86-2.09 (2H, m), 3.80-3.88 (1H, m), 3.96 (1H, br d, J=10.4 Hz), 4.40-4.49 (1H, m), 4.56-4.66 (1H, m), 5.25-5.43 (1H, m), 5.64 (1H, br d, J=10.4 Hz), 5.81-6.01 (1H, m), 6.08-6.17 (1H, m), 6.24-6.31 (1H, m), 6.36-6.62 (1H, m), 6.77-6.88 (1H, m), 7.26-7.45 (1H, m).

Preparative HPLC (NH₄HCO₃) method for the purification of Example 4. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 15-45% B in A over 10 min; Flow rate: 25 mL/min; Column temperature: 40° C.; Wavelength: 220 nm, 254 nm.

Preparative HPLC (formic acid) method for the purification of Example 4. Instrument: Shimadzu LC-20AP; Column: Shim-pack C18 150×25 mm×10 μm; Mobile phase: A=0.225% formic acid in water (v/v), B=MeCN; Gradient: 12-42% B in A over 10 min; Flow rate: 25 mL/min; Column temperature: 40° C.; Wavelength: 220 nm, 254 nm.

### Example 5: (1 R,2S,5S)—N-(4-Amino-1-((1 R,2S)-2-methylcyclopropyl)-3,4-dioxobutan-2-yl)-3-(isobutyryl-L-valyl)-6,6-dimethyl-3-azabicyclo[3.1.0]hexane-2-carboxamide

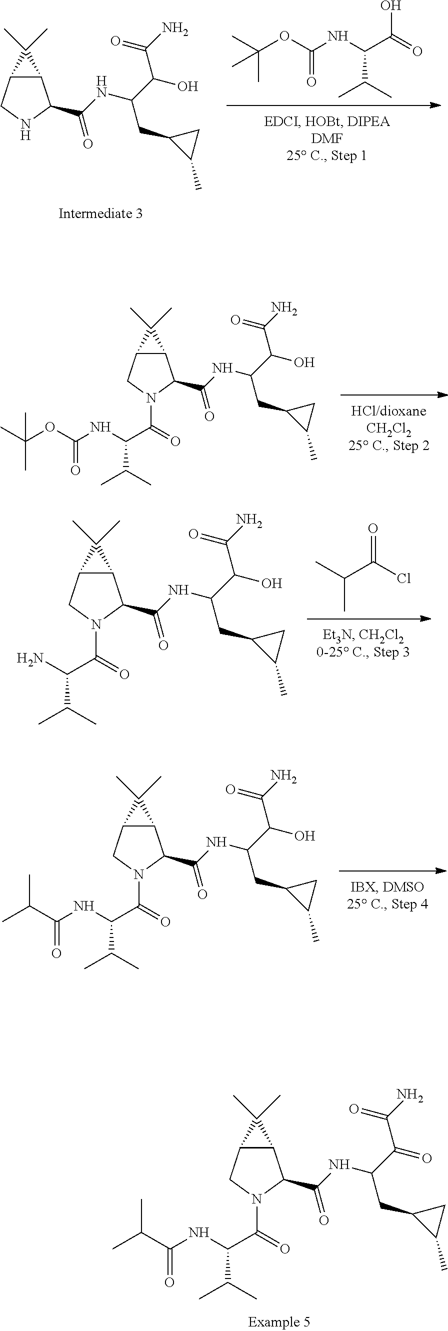

The title compound (16.0 mg, 0.03 mmol, yellow solid) was prepared from (1R,2S,5S)—N-(4-amino-3-hydroxy-1-((1 R,2S)-2-methylcyclopropyl)-4-oxobutan-2-yl)-6,6-dimethyl-3-azabicyclo[3.1.0]hexane-2-carboxamide hydrochloride (Intermediate 3, 0.37 g, 1.07 mmol) and (2S)-2-(tert-butoxycarbonylamino)-3-methyl-butanoic acid (232 mg, 1.07 mmol) using similar procedures to those detailed for Example 1 (Steps 1-4). Example 5 was purified by preparative HPLC (formic acid as additive).

LCMS (Method D2): m/z 477.3 (M+H), at 2.59 min.

¹H NMR: (400 MHz, CD₃OD) δ 0.10-0.30 (2H, m), 0.34-0.62 (2H, m), 0.88-1.14 (23H, m), 1.40-1.59 (2H, m), 1.94-2.17 (1H, m), 2.43-2.57 (1H, m), 3.89-4.09 (2H, m), 4.10-4.53 (3H, m).

Preparative HPLC (formic acid) method for the purification of Example 5. Instrument: Shimadzu LC-20AP; Column: Shim-pack C18 150×25 mm×10 μm; Mobile phase: A=0.225% formic acid in water (v/v), B=MeCN; Gradient: 25-55% B in A over 10 min; Flow rate: 25 mL/min; Column temperature: 25° C.; Wavelength: 220 nm, 254 nm.

### Example 6: (1 R,2S,5S)—N-(4-Amino-1-cyclopropyl-3,4-dioxobutan-2-yl)-3-((S)-3-cyclopropyl-2-isobutyramidopropanoyl)-6,6-dimethyl-3-azabicyclo[3.1.0]hexane-2-carboxamide

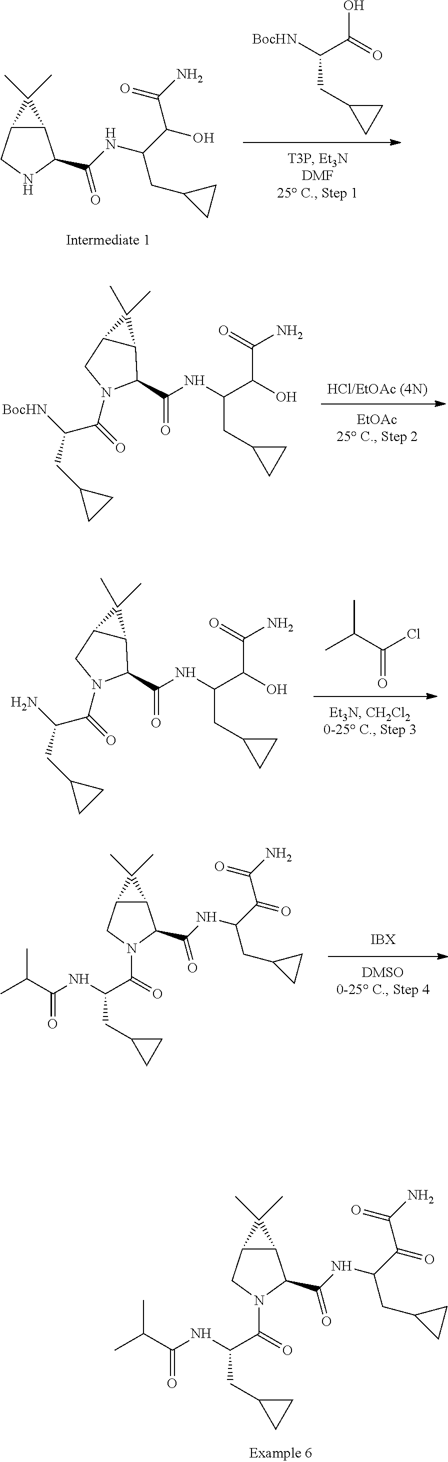

The title compound (76.6 mg, 0.16 mmol, white solid) was prepared from (1R,2S,5S)—N-[3-amino-1-(cyclopropylmethyl)-2-hydroxy-3-oxo-propyl]-6,6-dimethyl-3-azabicyclo[3.1.0]hexane-2-carboxamide (Intermediate 1, 380 mg, 1.29 mmol) and (2S)-2-(tert-butoxycarbonylamino)-3-cyclopropylpropanoic acid (383 mg, 1.67 mmol) using similar procedures to those detailed for Intermediate 1 (Step 9) and Example 1 (Steps 2-4). Example 6 was purified by preparative HPLC (NH₄HCO₃ as additive).

LCMS (Method D): m/z 475.6 (M+H), at 2.51 min.

¹H NMR: (400 MHz, CDCl₃) δ −0.02-0.14 (4H, m), 0.39-0.50 (4H, m), 0.62-0.77 (2H, m), 0.85-0.95 (3H, m), 1.03 (3H, s), 1.06-1.10 (3H, m), 1.11-1.13 (3H, m), 1.39-1.53 (2H, m), 1.56-1.64 (1H, m), 1.64-1.77 (2H, m), 1.77-1.89 (1H, m), 2.31-2.41 (1H, m), 3.80-3.86 (1H, m), 3.91-4.00 (1H, m), 4.43 (1H, d, J=11.2 Hz), 4.70-4.80 (1H, m), 5.29-5.43 (1H, m), 6.27-6.42 (1H, m), 6.48 (1H, br dd, J=18.8, 8.0 Hz), 6.85 (1H, br d, J=9.6 Hz), 7.19-7.38 (1H, m).

Preparative HPLC (NH₄HCO₃) method for the purification of Example 6. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 18-48% B in A over 10 min; Flow rate: 25 mL/min; Column temperature: 40° C.; Wavelength: 220 nm, 254 nm.

### Example 7: (1 R,2S,5S)—N-(4-Amino-1-cyclopropyl-3,4-dioxobutan-2-yl)-3-(isobutyryl-L-valyl)-6,6-dimethyl-3-azabicyclo[3.1.0]hexane-2-carboxamide

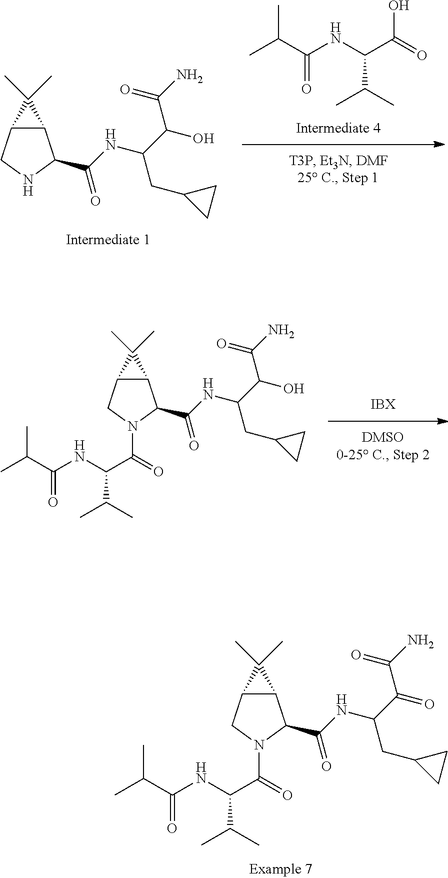

The title compound (16.1 mg, 0.03 mmol, white solid) was prepared from (1R,2S,5S)—N-[3-amino-1-(cyclopropylmethyl)-2-hydroxy-3-oxo-propyl]-6,6-dimethyl-3-azabicyclo[3.1.0]hexane-2-carboxamide (Intermediate 1, 180 mg, 0.61 mmol) and (2S)-3-methyl-2-(2-methylpropanoylamino)butanoic acid (Intermediate 4, 114 mg, 0.61 mmol) using similar procedures to those detailed for Intermediate 1 (Step 1) and Example 1 (Step 4). Example 7 was purified twice by preparative HPLC (formic acid as additive, followed by NH₄HCO₃ as additive).

LCMS (Method E): m/z 463.3 (M+H), at 2.73 min.

¹H NMR: (400 MHz, CDCl₃) δ 0.05-0.08 (1H, m), 0.11-0.13 (1H, m), 0.47-0.50 (2H, m), 0.70-0.75 (1H, m), 0.87 (3H, s), 0.92-0.96 (6H, m), 1.05 (3H, s), 1.12-1.16 (6H, m), 1.52 (1H, br s), 1.68-1.72 (1H, m), 1.76-1.88 (2H, m), 2.01-2.08 (1H, m), 2.36-2.41 (1H, m), 3.80-3.85 (1H, m), 3.98 (1H, br d, J=10.8 Hz), 4.47 (1H, d, J=12.0 Hz), 4.50-4.58 (1H, m), 5.35-5.43 (1H, m), 5.45-5.53 (1H, m), 5.98-6.06 (1H, m), 6.75-6.78 (1H, m), 7.21-7.44 (1H, m).

Preparative HPLC (formic acid) method for the purification of Example 7. Instrument: Shimadzu LC-20AP; Column: Unisil 3-100 C18 Ultra 150×50 mm×3 μm; Mobile phase: A=0.225% formic acid in water (v/v), B=MeCN; Gradient: 25-55% B in A over 10 min; Flow rate: 25 mL/min; Column temperature: 40° C.; Wavelength: 220 nm, 254 nm.

Preparative HPLC (NH₄HCO₃) method for the purification of Example 7. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 21-51% B in A over 9 min; Flow rate: 25 mL/min; Column temperature: 40° C.; Wavelength: 220 nm, 254 nm.

### Example 8: (1R,2S,5S)—N-(4-Amino-1-cyclopropyl-3,4-dioxobutan-2-yl)-6,6-dimethyl-3-(((S)-2-methylbutanoyl)-L-valyl)-3-azabicyclo[3.1.0]hexane-2-carboxamide

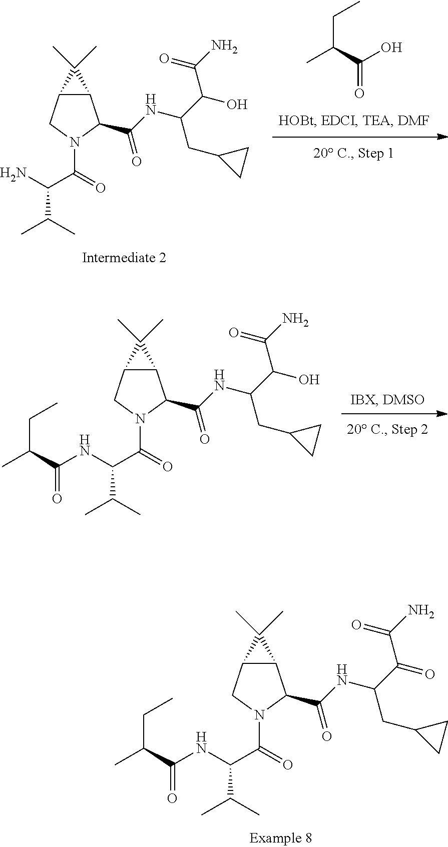

The title compound (41.5 mg, 0.06 mmol, white solid) was prepared from (1 R,2S,5S)—N-[3-amino-1-(cyclopropylmethyl)-2-hydroxy-3-oxo-propyl]-3-[(2S)-2-amino-3-methyl-butanoyl]-6,6-dimethyl-3-azabicyclo[3.1.0]hexane-2-carboxamide (Intermediate 2, 120 mg, 0.28 mmol)) and (2S)-2-methylbutanoic acid (28.4 mg, 0.28 mmol) using similar procedures to those detailed for Example 1 (Steps 1 and 4). Example 8 was purified by preparative HPLC (formic acid as additive).

LCMS (Method D2): m/z 477.0 (M+H), at 2.60 min.

¹H NMR: (400 MHz, CDCl₃) δ 0.00-0.20 (2H, m), 0.42-0.55 (2H, m), 0.68-0.76 (1H, m), 0.82-0.88 (6H, m), 0.91-0.99 (6H, m), 1.05 (3H, s), 1.14 (3H, d, J=6.8 Hz), 1.36-1.42 (1H, m), 1.48-1.54 (1H, m), 1.56-1.72 (2H, m), 1.75-1.89 (2H, m), 1.95-2.09 (1H, m), 2.11-2.22 (1H, m), 3.75-3.90 (1H, m), 4.01-4.04 (1H, m), 4.41-4.65 (2H, m), 5.39 (1H, br s), 5.60-5.83 (1H, m), 6.04-6.25 (1H, m), 6.78 (1H, br d, J=11.6 Hz), 7.42 (1H, br d, J=7.2 Hz).

Preparative HPLC (formic acid) method for the purification of Example 8. Instrument: Shimadzu LC-20AP; Column: Phenomenex luna C18 150×25 mm, 10 μm; Mobile phase: A=0.225% formic acid in water (v/v), B=MeCN; Gradient: 32-62% B in A over 10 min; Flow rate: 25 mL/min; Column temperature: 40° C.; Wavelength: 220 nm, 254 nm.

### Example 9: (1 R,2S,5S)—N-(4-Amino-1-cyclopropyl-3,4-dioxobutan-2-yl)-3-((S)-2-(cyclopropanecarboxamido)-2-cyclopropylacetyl)-6,6-dimethyl-3-azabicyclo[3.1.0]hexane-2-carboxamide

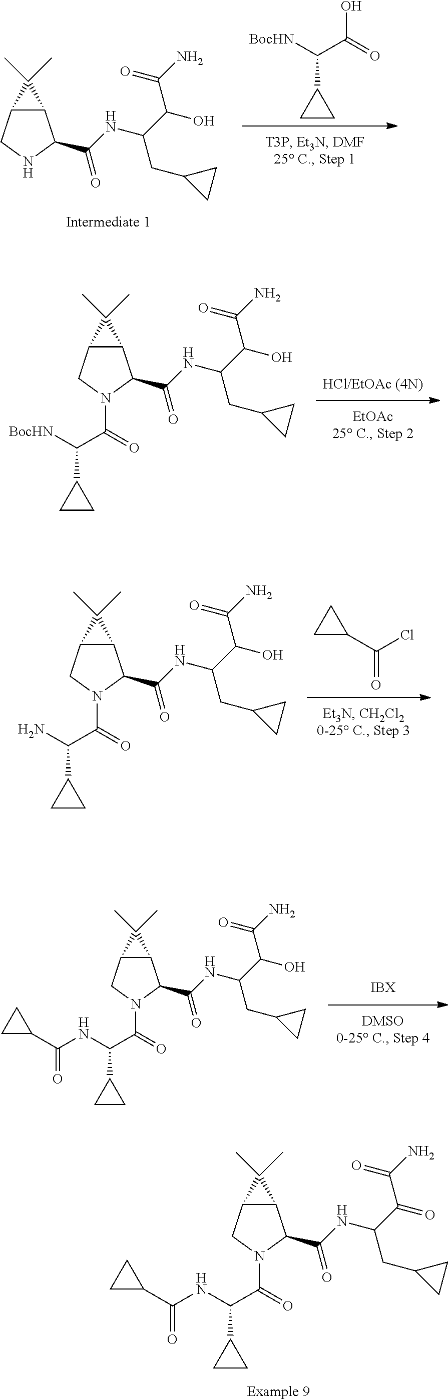

The title compound (10.9 mg, 0.02 mmol, white solid) was prepared from Intermediate 1 (560 mg, 1.90 mmol) and (2S)-2-(tert-butoxycarbonylamino)-2-cyclopropylacetic acid (612 mg, 2.84 mmol) using similar procedures to those detailed for Intermediate 1 (Step 1) and Example 1 (Steps 2-4). Example 9 was purified by preparative HPLC (NH₄HCO₃ as additive).

LCMS (Method D2): m/z 459.3 (M+H), at 2.44 min.

¹H NMR: (400 MHz, CDCl₃) δ 0.01-0.19 (2H, m), 0.40-0.61 (6H, m), 0.67-0.81 (3H, m), 0.86-0.94 (3H, m), 0.94-1.03 (2H, m), 1.03-1.10 (3H, m), 1.10-1.23 (1H, m), 1.36-1.42 (1H, m), 1.46-1.53 (1H, m), 1.66-1.71 (1H, m), 1.72-1.89 (2H, m), 3.76-3.99 (2H, m), 4.22-4.45 (1H, m), 4.45-4.66 (1H, m), 5.36-5.64 (1H, m), 6.34-6.59 (1H, m), 6.66-6.83 (1H, m).

Preparative HPLC (NH₄HCO₃) method for the purification of Example 9. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 15-45% B in A over 10 min; Flow rate: 25 mL/min; Column temperature: 40° C.; Wavelength: 220 nm, 254 nm.

### Example 10: (1 R,2S,5S)—N-(4-Amino-1-cyclopropyl-3,4-dioxobutan-2-yl)-3-((S)-2-cyclopropyl-2-isobutyramidoacetyl)-6,6-dimethyl-3-azabicyclo[3.1.0]hexane-2-carboxamide

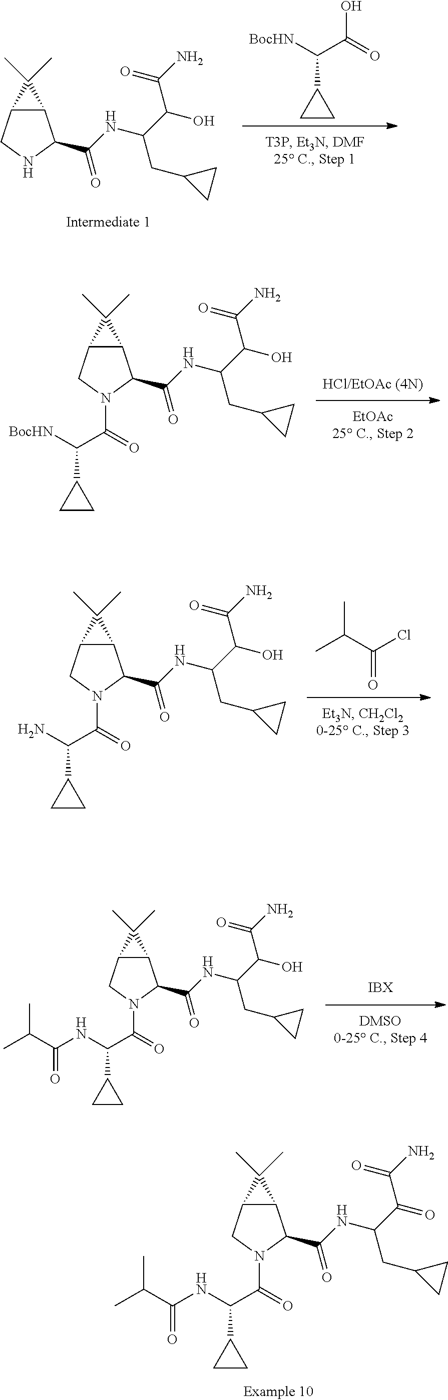

The title compound (20.2 mg, 0.04 mmol white solid) was prepared from Intermediate 1 (560 mg, 1.90 mmol) and (2S)-2-(tert-butoxycarbonylamino)-2-cyclopropylacetic acid (612 mg, 2.84 mmol) using similar procedures to those detailed for Intermediate 1 (Step 1) and Example 1 (Steps 2-4). Example 10 was purified by preparative HPLC (NH₄HCO₃ as additive).

LCMS (Method D2): m/z 461.5 (M+H), at 2.48 min.

¹H NMR: (400 MHz, CDCl₃) δ 0.01-0.06 (1H, m), 0.09-0.14 (1H, m), 0.40-0.52 (6H, m), 0.68-0.73 (1H, m), 0.89 (3H, s), 1.04 (3H, s), 1.13 (6H, dd, J=15.6, 6.8 Hz), 1.47-1.53 (1H, m), 1.68 (1H, dd, J=7.6, 2.0 Hz), 1.78-1.83 (2H, m), 1.84-1.90 (1H, m), 2.34-2.42 (1H, m), 3.82-3.90 (2H, m), 4.36 (1H, br t, J=7.8 Hz), 4.46 (1H, d, J=16.8 Hz), 5.36-5.45 (1H, m), 5.77-5.91 (1H, m), 6.32-6.34 (1H, m), 6.75-6.87 (1H, m), 7.22-7.34 (1H, m).

Preparative HPLC (NH₄HCO₃) method for the purification of Example 10. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 15-45% B in A over 10 min; Flow rate: 25 mL/min; Column temperature: 40° C.; Wavelength: 220 nm, 254 nm.

### Example 11: (1 R,2S,5S)—N-(4-(Benzylamino)-1-cyclopropyl-3,4-dioxobutan-2-yl)-3-(isobutyryl-L-valyl)-6,6-dimethyl-3-azabicyclo[3.1.0]hexane-2-carboxamide

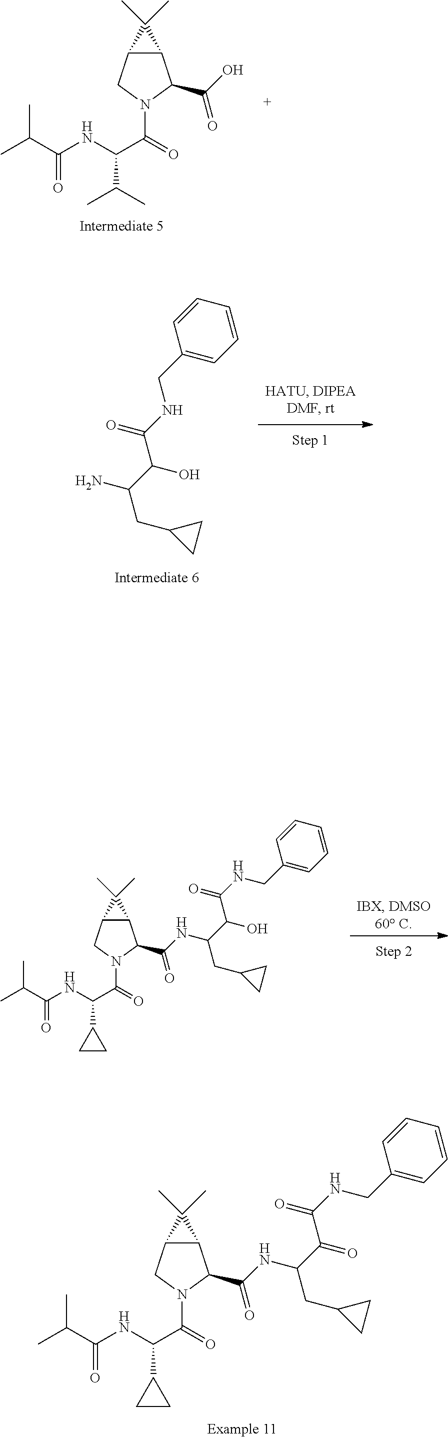 [SMILES: CC(C)C(=O)N[C@H](C(=O)N1C[C@H]2[C@@H]([C@H]1C(=O)O)C2(C)C)C(C)C ; NC(CC1CC1)C(O)C(=O)NCc1ccccc1 ; CC(C)C(=O)N[C@H](C(=O)N1C[C@H]2[C@@H]([C@H]1C(=O)NC(CC1CC1)C(O)C(=O)NCc1ccccc1)C2(C)C)C1CC1 ; "IBX, DMSO60° C." ; CC(C)C(=O)N[C@H](C(=O)N1C[C@H]2[C@@H]([C@H]1C(=O)NC(CC1CC1)C(=O)C(=O)NCc1ccccc1)C2(C)C)C1CC1]

Step 1: HATU (1.23 g, 3.23 mmol) was added to a solution of (1 R,2S,5S)-3-(isobutyryl-L-valyl)-6,6-dimethyl-3-azabicyclo[3.1.0]hexane-2-carboxylic acid (Intermediate 5, 0.70 g, 2.16 mmol) in DMF (10 mL) and the reaction mixture was stirred at rt for 10 min. 3-Amino-N-benzyl-4-cyclopropyl-2-hydroxybutanamide (Intermediate 6, 0.53 g, 2.16 mmol) and DIPEA (1.11 mL, 6.47 mmol) were added and after stirring at rt for 18 h the reaction mixture was partitioned between cold water (150 mL) and EtOAc (70 mL). The phases were separated and the aqueous layer was extracted with EtOAc (3×70 mL). The combined organic phases were dried (Na₂SO₄), filtered and concentrated in vacuo. Purification by reverse phase gradient column chromatography (C18 silica), eluting with 0-60% MeCN in water yielded (1 R,2S,5S)—N-((2S)-4-(benzylamino)-1-cyclopropyl-3-hydroxy-4-oxobutan-2-yl)-3-(isobutyryl-L-valyl)-6,6-dimethyl-3-azabicyclo[3.1.0]hexane-2-carboxamide (0.59 g, 1.06 mmol) as a white solid.

LCMS (Method G): m/z 555.9 (M+H), at 2.20, 2.25, 2.38 min.

Step 2: IBX (0.40 g, 1.44 mmol) was added to a solution of (1 R,2S,5S)—N-((2S)-4-(benzylamino)-1-cyclopropyl-3-hydroxy-4-oxobutan-2-yl)-3-(isobutyryl-L-valyl)-6,6-dimethyl-3-azabicyclo[3.1.0]hexane-2-carboxamide (0.40 g, 0.72 mmol) in DMSO (5 mL) at rt and the reaction mixture was stirred at 60° C. for 2 h. Water (50 mL) and EtOAc (30 mL) were added, the phases were separated and the aqueous phase was extracted with EtOAc (2×50 mL). The combined organic phases were dried (Na₂SO₄), filtered and concentrated in vacuo. Purification by reverse phase gradient column chromatography (C18 silica) eluting with 0-60% MeCN in water yielded Example 11 (0.22 g, 0.40 mmol) as a white solid.

LCMS (Method H): m/z 553.3 (M+H), at 7.70, 7.95 and 8.29 min.

¹H NMR: (400 MHz, DMSO-d₆) δ 0.08-0.07 (m, 2H), 0.41-0.35 (m, 2H), 0.77-0.69 (m, 9H), 1.01-0.83 (m, 10H), 1.32 (d, 1H, J=7.6 Hz), 1.49-1.40 (m, 2H), 1.79-1.69 (m, 1H), 1.91-1.90 (m, 1H), 2.45-2.41 (m, 1H), 3.75-3.72 (m, 1H), 3.89 (d, 1H, J=10.4 Hz), 4.11 (t, 1H, J=9.2 Hz), 4.43-4.25 (m, 3H), 5.10-5.05 (m, 1H), 7.33-7.24 (m, 5H), 7.97 (d, 1H, J=8.4 Hz), 8.50 (d, 1H, J=6.4 Hz), 9.26 (t, 1H, J=6.0 Hz).

### Example 12: (1 R,2S,5S)—N-(1-Cyclopropyl-4-(cyclopropylamino)-3,4-dioxobutan-2-yl)-3-(isobutyryl-L-valyl)-6,6-dimethyl-3-azabicyclo[3.1.0]hexane-2-carboxamide

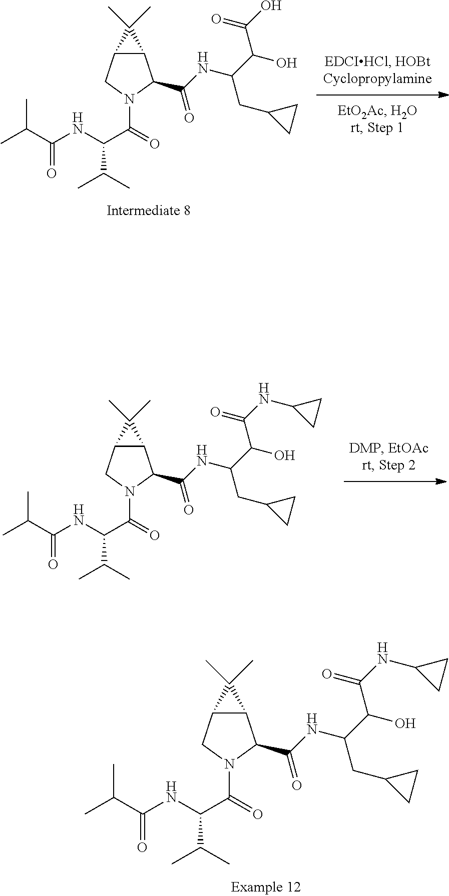 [SMILES: CC(C)C(=O)N[C@H](C(=O)N1C[C@H]2[C@@H]([C@H]1C(=O)NC(CC1CC1)C(O)C(=O)O)C2(C)C)C(C)C ; CC(C)C(=O)N[C@H](C(=O)N1C[C@H]2[C@@H]([C@H]1C(=O)NC(CC1CC1)C(O)C(=O)NC1CC1)C2(C)C)C(C)C ; CC(C)C(=O)N[C@H](C(=O)N1C[C@H]2[C@@H]([C@H]1C(=O)NC(CC1CC1)C(O)C(=O)NC1CC1)C2(C)C)C(C)C]

Step 1: EDCl·HCl (0.19 g, 1.03 mmol) and HOBt (0.11 g, 0.86 mmol) were added to a solution of 4-cyclopropyl-2-hydroxy-3-((1 R,2S,5S)-3-(isobutyryl-L-valyl)-6,6-dimethyl-3-azabicyclo [3.1.0]hexane-2-carboxamido)butanoic acid (Intermediate 8, 0.40 g, 0.86 mmol) and cyclopropylamine (59 mg, 1.03 mmol) in EtOAc (4 mL) and H₂O (1 mL) at 0° C. and the reaction mixture was stirred at rt for 3 h before concentration in vacuo. Purification by reverse phase gradient flash column chromatography (C18 silica), eluting with 0-46% MeCN in water yielded (1 R,2S,5S)—N-(1-cyclopropyl-4-(cyclopropylamino)-3-hydroxy-4-oxobutan-2-yl)-3-(isobutyryl-L-valyl)-6,6-dimethyl-3-azabicyclo[3.1.0]hexane-2-carboxamide (0.25 g, 0.50 mmol) as an off-white solid.

LCMS (Method 1): m/z 505.6 (M+H), at 1.51, 1.57, 1.66 min

Step 2: DMP (0.31 g, 0.74 mmol) was added to a solution of (1 R,2S,5S)—N-(1-cyclopropyl-4-(cyclopropylamino)-3-hydroxy-4-oxobutan-2-yl)-3-(isobutyryl-L-valyl)-6,6-dimethyl-3-azabicyclo[3.1.0]hexane-2-carboxamide (0.25 g, 0.49 mmol) in EtOAc (3 mL) and the reaction mixture was stirred at rt for 48 h. After filtering through celite and washing with EtOAc (50 mL) the filtrate was concentrated in vacuo. Purification by reverse phase gradient flash column chromatography (C18 silica), eluting with 0-65% MeCN in water with 0.1% formic acid as a modifier yielded Example 12 (31 mg, 0.06 mmol) as an off-white solid.

LCMS (Method 1): m/z 503.5 (M+H), at 1.48, 1.75 min

¹H NMR: (400 MHz, DMSO-d₆) δ 0.08-0.03 (m, 2H), 0.42-0.37 (m, 2H), 0.56 (s, 2H), 0.65-0.63 (d, 2H, J=7.2 Hz), 0.75-0.74 (m, 9H), 1.11-0.82 (m, 10H), 1.48-1.23 (m, 3H), 1.89-1.69 (m, 1H), 1.92-1.90 (m, 1H), 2.50-2.33 (m, 1H), 2.74-2.60 (m, 1H), 3.74-3.70 (m, 1H), 3.91-3.88 (m, 1H), 4.12-4.07 (m, 1H), 4.29 (s, 1H), 5.05-5.01 (m, 1H), 7.98-7.96 (d, 1H, J=8.4 Hz), 8.47-8.46 (d, 1H, J=6.4 Hz), 8.75-8.74 (d, 1H, J=4.8 Hz).

### Example 26: (1 R,2S,5S)—N-(4-Amino-1-cyclopropyl-3,4-dioxobutan-2-yl)-3-((S)-2-isobutyramido-3-methyl-3-phenylbutanoyl)-6,6-dimethyl-3-azabicyclo[3.1.0]hexane-2-carboxamide

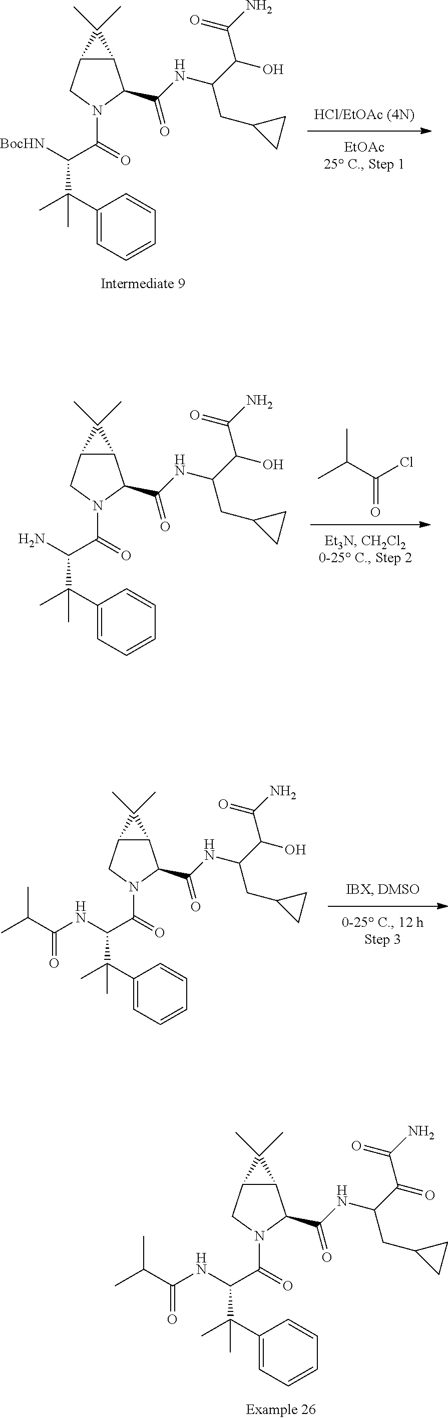

The title compound (18.2 mg, 0.03 mmol white solid) was prepared from Intermediate 9 (31.0 mg, 0.05 mmol) using similar procedures to those detailed for Example 1 (Steps 2-4). Example 26 was purified by preparative HPLC (NH₄HCO₃ as additive).

LCMS (Method J): m/z 539.3 (M+H), at 2.80 min.

¹H NMR: (400 MHz, CDCl₃) δ 0.03-0.22 (2H, m), 0.43-0.57 (2H, m), 0.71-0.78 (3H, m), 0.78-0.89 (1H, m), 0.94-0.98 (3H, m), 1.02-1.08 (6H, m), 1.27-1.32 (1H, m), 1.40-1.48 (6H, m), 1.52-1.62 (1H, m), 1.80-1.91 (2H, m), 2.27-2.35 (1H, m), 2.83-2.89 (1H, m), 3.69-3.72 (1H, m), 4.32-4.36 (1H, m), 4.97-5.01 (1H, m), 5.27-5.43 (1H, m), 5.72-5.88 (1H, m), 6.09-6.20 (1H, m), 6.81 (1H, brs), 7.17-7.23 (1H, m), 7.26-7.34 (2H, m), 7.38 (2H, d, J=7.6 Hz), 7.41-7.52 (1H, m).

Preparative HPLC (NH₄HCO₃) method for the purification of Example 26. Instrument: Shimadzu LC-20AP; Column: Waters Xbridge 150×25 mm×5 μm; Mobile phase: A=10 mM aqueous NH₄HCO₃ solution (v/v), B=MeCN; Gradient: 30-60% B in A over 10 min; Flow rate: 25 mL/min; Column temperature: 40° C.; Wavelength: 220 nm, 254 nm.

### Example 28: (1 R,2S,5S)—N-(4-(Azetidin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide

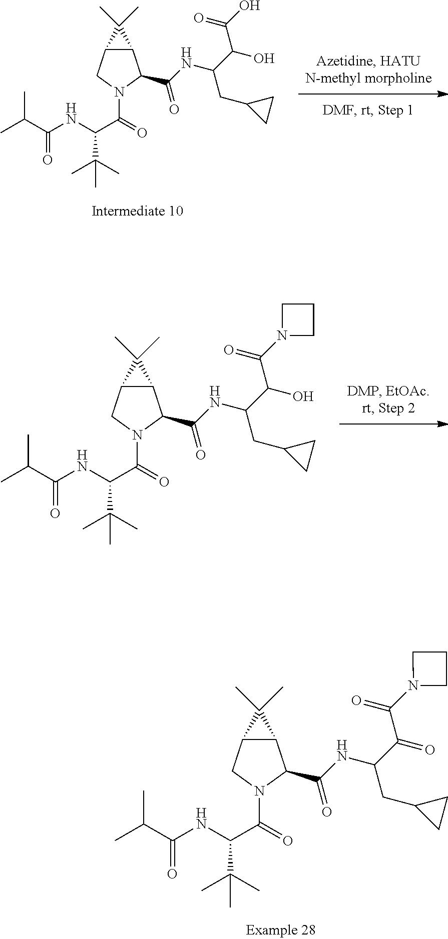 [SMILES: CC(C)C(=O)N[C@H](C(=O)N1C[C@H]2[C@@H]([C@H]1C(=O)NC(CC1CC1)C(O)C(=O)O)C2(C)C)C(C)(C)C ; CC(C)C(=O)N[C@H](C(=O)N1C[C@H]2[C@@H]([C@H]1C(=O)NC(CC1CC1)C(O)C(=O)N1CCC1)C2(C)C)C(C)(C)C ; CC(C)C(=O)N[C@H](C(=O)N1C[C@H]2[C@@H]([C@H]1C(=O)NC(CC1CC1)C(=O)C(=O)N1CCC1)C2(C)C)C(C)(C)C]

Step 1: N-Methyl morpholine (0.17 mL, 1.59 mmol) was added to a solution of 4-cyclopropyl-2-hydroxy-3-((1 R,2S,5S)-3-((S)-2-isobutyramido-3,3-dimethylbutanoyl)-6,6-dimethyl-3-azabicyclo[3.1.0]hexane-2-carboxamido)butanoic acid (Intermediate 10, 0.19 g, 0.40 mmol) and azetidine (32 mg, 0.48 mmol) in DMF (2.8 mL) at 0° C. and the reaction mixture was then stirred at rt for 4 h. Water (80 mL) was added and the mixture was extracted with EtOAc (3×25 mL). The combined organic layers were dried (Na₂SO₄) and concentrated in vacuo. Purification by reverse phase gradient flash column chromatography (Silica C18), eluting with 0% to 60% MeCN in water yielded (1 R,2S,5S)—N-(4-(azetidin-1-yl)-1-cyclopropyl-3-hydroxy-4-oxobutan-2-yl)-3-((S)-2-isobutyramido-3,3-dimethylbutanoyl)-6,6-dimethyl-3-azabicyclo[3.1.0]hexane-2-carboxamide (0.15 g, 0.29 mmol) as a brown solid.

LCMS (Method F3): m/z 519.4 (M+H), at 1.58, 1.65 and 1.70 min.

Step 2: Dess-Martin periodinane (0.17 g, 0.41 mmol) was added to a solution of (1 R,2S,5S)—N-(4-(azetidin-1-yl)-1-cyclopropyl-3-hydroxy-4-oxobutan-2-yl)-3-((S)-2-isobutyramido-3,3-dimethylbutanoyl)-6,6-dimethyl-3-azabicyclo[3.1.0]hexane-2-carboxamide (0.14 g, 0.27 mmol) in EtOAc (3 mL) and the reaction mixture was stirred at rt for 16 h before being filtered through a celite bed and washed with EtOAc (100 mL). The filtrate was concentrated in vacuo, purification by reverse phase gradient flash column chromatography (Silica C18), eluting with 0% to 90% MeCN in (water+0.1% formic acid) yielded Example 28, (1R,2S,5S)—N-(4-(azetidin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide (57 mg, 0.11 mmol) as a white solid.

LCMS (Method K): m/z 517.0 (M+H), at 7.49 min.

¹H NMR: (400 MHz, DMSO-d₆) δ 0.03-0.11 (m, 2H), 0.35-0.43 (m, 2H), 0.80-1.03 (m, 22H), 1.29-1.31 (m, 1H), 1.43-1.50 (m, 2H), 1.69-1.71 (m, 1H), 2.23-2.27 (t, 2H, J=7.8 Hz), 2.54-2.59 (m, 1H), 3.77-3.85 (m, 2H), 3.88-4.00 (m, 2H), 4.20-4.33 (m, 3H), 4.39-4.41 (d, 1H, J=9.6 Hz), 4.72-4.77 (m, 1H), 7.74-7.76 (d, 1H, J=9.2 Hz), 8.52-8.53 (d, 1H, J=6.4 Hz).

### Example 34: (1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide

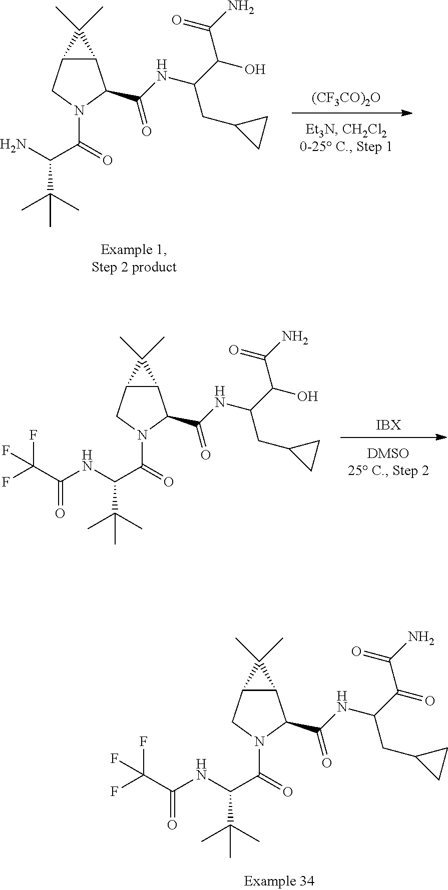

Step 1: To a mixture of (1 R,2S,5S)—N-[3-amino-1-(cyclopropylmethyl)-2-hydroxy-3-oxo-propyl]-3-[(2S)-2-amino-3,3-dimethylbutanoyl]-6,6-dimethyl-3-azabicyclo[3.1.0]hexane-2-carboxamide (Example 1, Step 2 product, 400 mg, 0.98 mmol) in CH₂Cl₂ (5 mL) was added Et₃N (0.20 mL, 1.47 mmol,) and a solution of TFAA (0.20 mL, 1.47 mmol, 0.20 mL) in CH₂Cl₂ (2 mL) dropwise at 0° C. The reaction mixture was stirred at 25° C. for 2 h before the addition of H₂O (40 mL). After extraction with ethyl acetate (20 mL×3) the combined organic phases were washed with brine (30 mL×3), dried with anhydrous Na₂SO₄, filtered and concentrated in vacuo. Purification by preparative HPLC (formic acid as additive) yielded (1 R,2S,5S)—N-(4-amino-1-cyclopropyl-3-hydroxy-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (170 mg, 0.34 mmol) as a white solid.

LCMS (Method A): m/z 505.2 (M+H), at 0.81 min.

¹H NMR: (400 MHz, MeOD-d₄) δ −0.08-0.21 (2H, m), 0.26-0.53 (2H, m), 0.64-1.14 (16H, m), 1.16-2.32 (4H, m), 3.60-4.73 (6H, m).

Preparative HPLC (formic acid) method for the purification of Step 1. Instrument: Shimadzu LC-20AP; Column: Unisil 3-100 C18 Ultra 150×50 mm×3 μm; Mobile phase: A=0.225% formic acid in water (v/v), B=MeCN; Gradient: 30-60% B in A over 10 min; Flow rate: 25 mL/min; Column temperature: 40° C.; Wavelength: 220 nm, 254 nm.

Step 2: IBX (305 mg, 1.09 mmol) was added to a solution of (1R,2S,5S)—N-(4-amino-1-cyclopropyl-3-hydroxy-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (220 mg, 0.44 mmol) in DMSO (3 mL) and the reaction mixture was stirred at 25° C. for 2 h before the addition of H₂O (20 mL) and extraction with ethyl acetate (20 mL×3). The combined organic phases were washed with brine (30 mL×3), dried with anhydrous Na₂SO₄, filtered and concentrated in vacuo. Purification by preparative HPLC (formic acid as additive) yielded (1 R,2S,5S)—N-(4-amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (195 mg, 0.39 mmol) as a white solid.

LCMS (Method J): m/z 503.3 (M+H), at 3.04 min.

¹H NMR: (400 MHz, DMSO-d₆) δ −0.14-0.16 (2H, m), 0.24-0.49 (2H, m), 0.56-1.20 (16H, m), 1.34-1.77 (4H, m), 3.47-3.99 (2H, m), 4.17-4.64 (2H, m), 4.74-5.38 (1H, m), 7.66-7.85 (1H, m), 7.86-8.11 (1H, m), 8.26-8.74 (1H, m), 9.15-9.59 (1H, m).

Preparative HPLC (formic acid) method for the purification of Step 1. Instrument: Shimadzu LC-20AP; Column: Unisil 3-100 C18 Ultra 150×50 mm×3 μm; Mobile phase: A=0.225% formic acid in water (v/v), B=MeCN; Gradient: 33-63% B in A over 10 min; Flow rate: 25 mL/min; Column temperature: 40° C.; Wavelength: 220 nm, 254 nm.

### Example 35: (1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide

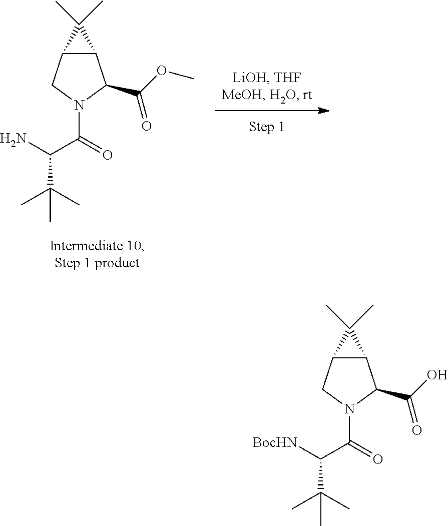 [SMILES: COC(=O)[C@@H]1[C@@H]2[C@H](CN1C(=O)[C@@H](N)C(C)(C)C)C2(C)C ; "LiOH, THFMeOH, H2O, rt" ; CC(C)(C)OC(=O)N[C@H](C(=O)N1C[C@H]2[C@@H]([C@H]1C(=O)O)C2(C)C)C(C)(C)C]

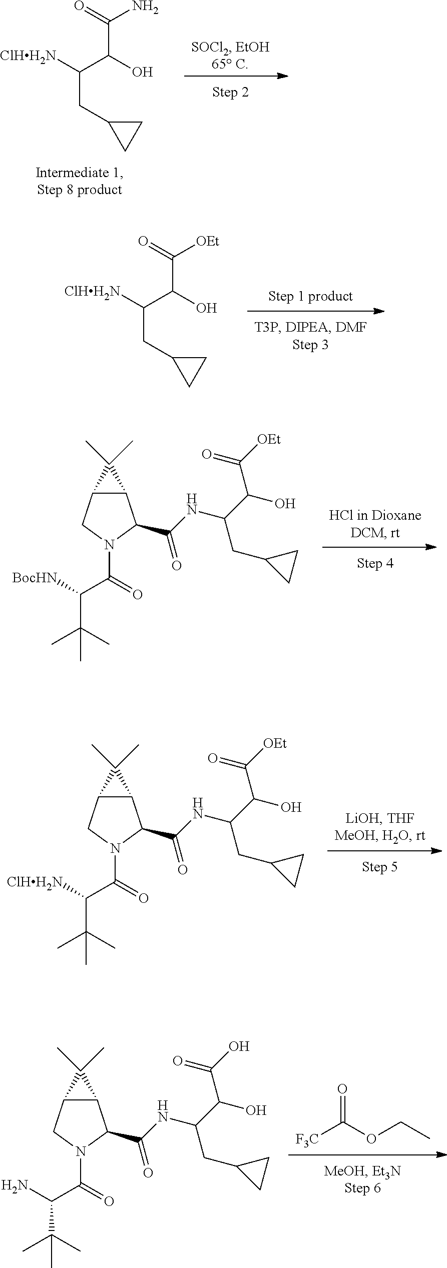

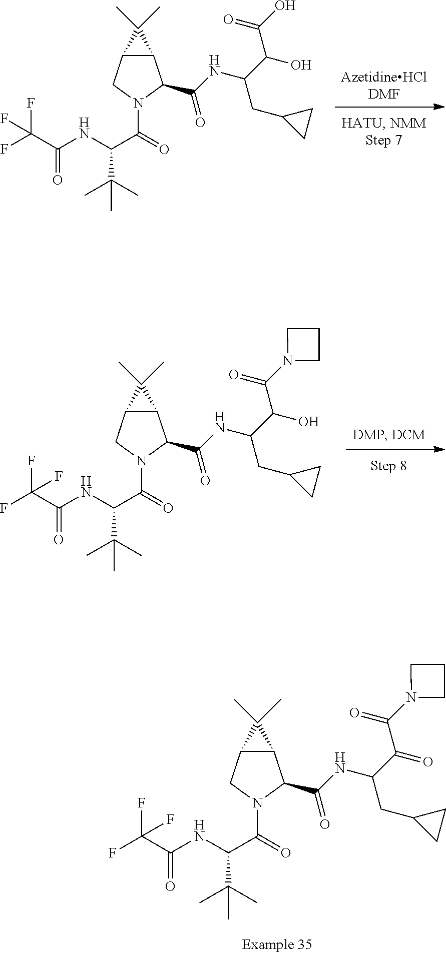

Step 1: H (154 mg, 6.37 mmol) was added to a suspension of methyl (1 R,2S,5S)-3-((S)-2-((tert-butoxycarbonyl)amino)-3,3-dimethylbutanoyl)-6,6-dimethyl-3-azabicyclo[3.1.0]hexane-2-carboxylate (Intermediate 10, Step 1 product, 2.50 g, 6.37 mmol) in THF/MeOH/H₂O (32 mL, 10:3:3) and the mixture stirred at rt for 3 h. Water (10 mL) and MTBE (10 mL) were added and the phases were separated. The aqueous layer was acidified with 1.5 N HCl to approximately pH 2-3 and extracted with DCM (2×30 mL). The combined organic phases were dried over anhydrous Na₂SO₄ and concentrated in vacuo to yield (1 R,2S,5S)-3-((S)-2-((tert-butoxycarbonyl)amino)-3,3-dimethylbutanoyl)-6,6-dimethyl-3-azabicyclo[3.1.0]hexane-2-carboxylic acid (2.12 g, 5.60 mmol) as a white solid which was used in the next step without further purification.

LCMS (Method L): m/z 369.3 (M+H), at 2.26 min.

¹H NMR: (400 MHz, DMSO-d₆) δ 12.53 (br s, 1H), 6.68 (d, J=9.6 Hz, 1H), 4.13 (s, 1H), 4.05 (d, J=9.6 Hz, 1H), 3.91 (d, J=10.4 Hz, 1H), 3.79-3.74 (m, 1H), 1.51-1.47 (m, 1H), 1.41-1.37 (m, 1H), 1.35 (s, 9H), 1.01 (s, 3H), 0.94 (s, 9H), 0.84 (s, 3H).

Step 2: SOCl₂ (1.15 mL, 15.6 mmol) was added to a stirred solution of 3-amino-4-cyclopropyl-2-hydroxybutanamide (2.50 g, 15.6 mmol) in EtOH (20 mL) at 0° C. and the reaction mixture was then stirred at 65° C. for 15 h. Concentration in vacuo and azeotroping with toluene (2×20 mL) yielded crude ethyl 3-amino-4-cyclopropyl-2-hydroxybutanoate (2.10 g) as a brown semi-solid which was used in the following step without further purification.

LCMS (Method L): m/z 188.3 (M+H), at 1.03 min.

Step 3: To a suspension of (1 R,2S,5S)-3-((S)-2-((tert-butoxycarbonyl)amino)-3,3-dimethylbutanoyl)-6,6-dimethyl-3-azabicyclo[3.1.0]hexane-2-carboxylic acid (2.45 g, 6.25 mmol) and ethyl 3-amino-4-cyclopropyl-2-hydroxybutanoate (1.65 g, 7.50 mmol) in DCM (20 mL) was added DIPEA (3.37 mL, 18.8 mmol) at rt followed by T3P (50% in EtOAc) (5.58 mL, 9.38 mmol) at 0° C. The resultant reaction mixture was stirred at rt for 3 h, 10% aqueous NaHCO₃ was added at rt and the mixture was extracted with DCM (2×25 mL). The combined organic phases were dried over anhydrous Na₂SO₄ and concentrated in vacuo. Purification by gradient flash column chromatography on silica, eluting with 0-50% EtOAc in petroleum ether yielded ethyl 3-((1 R,2S,5S)-3-((S)-2-((tert-butoxycarbonyl)amino)-3,3-dimethylbutanoyl)-6,6-dimethyl-3-azabicyclo[3.1.0]hexane-2-carboxamido)-4-cyclopropyl-2-hydroxybutanoate (2.30 g, 4.19 mmol) as a pale yellow gum.

LCMS (Method L): m/z 538.4 (M+H), at 2.43-2.47 min.

¹H NMR: (400 MHz, DMSO-d₆) δ 7.90 (d, J=8.8 Hz, 1H), 6.57 (d, J=9.2 Hz, 1H), 5.57-5.50 (m, 1H), 4.27 (d, J=2.8 Hz, 1H), 4.11-4.09 (m, 1H), 4.04-4.00 (m, 4H), 3.95-3.85 (m, 1H), 3.82-3.71 (m, 1H), 1.40 (s, 9H), 1.18-1.13 (m, 6H), 1.05-0.95 (m, 4H), 0.93-0.83 (m, 10H), 0.72-0.61 (m, 1H), 0.35-0.09 (m, 2H), 0.15-0.15 (m, 2H).

Step 4: 4M HCl in 1,4-dioxane (5 mL, 20 mmol) was added a solution of ethyl 3-((1 R,2S,5S)-3-((S)-2-((tert-butoxycarbonyl)amino)-3,3-dimethylbutanoyl)-6,6-dimethyl-3-azabicyclo[3.1.0]hexane-2-carboxamido)-4-cyclopropyl-2-hydroxybutanoate (2.20 g, 4.01 mmol) in DCM (10 mL) at 0° C. and the reaction mixture was stirred at rt for 3 h. The supernatant layer was decanted from the reaction mixture, and the solid gummy residue was triturated with MTBE (10 mL). The MTBE layer was decanted and the residue was dried in vacuo to yield crude ethyl 3-((1 R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoate hydrochloride (1.80 g) as an off white solid, which was used in the next step without further purification.

LCMS (Method L): m/z 438.4 (M+H), at 1.42-1.79 min.

Step 5: LiOH (296 mg, 12.2 mmol) was added to a suspension of ethyl 3-((1 R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoate hydrochloride (1.45 g, 2.45 mmol) in THF/MeOH/H₂O (16 mL, 10:3:3) at rt. After stirring at rt for 3 h water (5 mL) was added and the mixture acidified with 1.5 N aqueous HCl to approximately pH 2-3 and the reaction mixture was concentrated in vacuo and azeotroped with toluene (3×20 mL) to yield crude 3-((1 R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoic acid hydrochloride (2.2 g) as a light brown solid which was used in the next step without further purification.

LCMS (Method L): m/z 410.3 (M+H), at 1.05-1.53 min.

Step 6: Trimethylamine (1.04 mL, 7.38 mmol) and ethyl 2,2,2-trifluoroacetate (0.48 mL, 4.92 mmol) were added to a solution of 3-((1 R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoic acid hydrochloride (2.4 g, 2.46 mmol) in MeOH (20 mL) at 0° C. and the reaction mixture stirred at rt for 16 h. The reaction mixture was then acidified with 4M HCl in 1,4-dioxane (3 mL, 12 mmol) and concentrated in vacuo before addition of water (10 mL) and extraction with EtOAc (2×25 mL). The combined organic phases were dried over anhydrous sodium sulphate, filtered, concentrated in vacuo and azeotroped with toluene (3×20 mL) to yield crude 4-cyclopropyl-3-((1 R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoic acid (1.20 g) as an off white solid, which was used in the next step without further purification.

LCMS (Method L): m/z 506.4 (M+H), at 2.03-2.09 min.

Step 7: N-Methylmorpholine (NMM, 1.37 mL, 12.4 mmol) was added to a stirred solution of azetidine hydrochloride (467 mg, 4.94 mmol) and 4-cyclopropyl-3-((1 R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoic acid (1.42 g, 2.47 mmol) in DMF (5 mL) and the reaction mixture was stirred for 5 min at rt. HATU (1.42 g, 3.71 mmol) was added at 0° C. and the reaction mixture was stirred at rt for 2 h before the addition of water (2 mL) and concentration in vacuo. Purification by gradient reverse phase flash column chromatography on silica C18, eluting with 0% to 70% MeCN in water yielded (1 R,2S,5S)—N-(4-(azetidin-1-yl)-1-cyclopropyl-3-hydroxy-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (0.75 g, 1.18 mmol) as an off white solid.

LCMS (Method L): m/z 545.3 (M+H), at 2.06-2.14 min.

¹H NMR: (400 MHz, DMSO-d₆) δ 9.43-9.31 (m, 1H), 7.94-7.71 (m, 1H), 5.22-5.01 (m, 1H), 4.43-4.31 (m, 1H), 4.30-4.17 (m, 3H), 4.15-3.70 (m, 4H), 2.28-2.18 (m, 2H), 1.57-1.48 (m, 2H), 1.31-1.22 (m, 2H), 1.21-0.82 (m, 17H), 0.76-0.65 (m, 1H), 0.39-0.17 (m, 2H), 0.15-0.19 (m, 2H).

Step 8: Dess-Martin periodinane (1.17 g, 2.75 mmol) was added to a solution of (1R,2S,5S)—N-(4-(azetidin-1-yl)-1-cyclopropyl-3-hydroxy-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (0.50 g, 0.92 mmol) in EtOAc (10 mL) at rt and the reaction mixture was stirred at rt for 2 h before EtOAc (10 mL) was added. The mixture was filtered through celite and the residue rinsed with EtOAc (50 mL). The filtrate was concentrated in vacuo and combined with the crude filtrate from another batch prepared from 0.25 g of (1R,2S,5S)—N-(4-(azetidin-1-yl)-1-cyclopropyl-3-hydroxy-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide). Purification by gradient reverse phase flash column chromatography on silica C18, eluting with 0% to 80% MeCN in (H₂O+0.1% TFA) yielded (1R,2S,5S)—N-(4-(azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (Example 35, 185 mg, 0.33 mmol) as a white solid.

LCMS (Method L): m/z 543.3 (M+H), at 2.34 min.

¹H NMR: (400 MHz, DMSO-d₆) δ 9.40-9.35 (m, 1H), 8.73-8.58 (m, 1H), 4.81-4.51 (m, 1H), 4.44-4.18 (m, 2H), 3.97-3.69 (m, 4H), 2.34-2.22 (m, 2H), 1.73-1.34 (m, 3H), 1,34-1.28 (m, 1H), 1.06-0.76 (m, 18H), 0.42-0.36 (m, 2H), 0.18-0.05 (m, 2H).

Examples 13-25, 27, 29-33 were synthesized using procedures similar to those detailed above.

### Biological Activity

### Construct Design of SARS-CoV-2 Mpro

The SARS CoV-2-Mpro (Main Protease/3C-like protease, UniProt ID: P0DTD1) protein sequence, up to and including its autocleavage boundaries, as well as the preceding N-terminal 5 amino acid residues, including the P1 glutamine residue, were codon optimised for E. coli expression and cloned into pET26b (Merck, #US169862-3) or pGEX6P1 (Fisher Scientific, #10350355) vectors using BamHI and XhoI sites. The expression constructs thus featured a native viral N-terminal sequence, as well as a C-terminal modified 3C-protease cleavage site (LEVLFQGK), with an alternative lysine residue at the P2′ position, followed by a polyhistidine (His-8) tag.

### Protein Expression and Protein Purification

Chemically competent BL21(DE3)-RIL E. coli (Agilent, #230240) cells were transformed with the relevant coronavirus Mpro construct and grown overnight at 37° C. on LB agar plates supplemented with the appropriate antibiotics. All culture steps were performed at 37° C. unless otherwise stated. A scraping of colonies was grown in 15 mL of antibiotic supplemented LB media, for a period of approximately 2 hours, taking care not to exceed an optical density (OD) density of 2.0 as measured in a spectrophotometer at 600 nm. This preculture was used to inoculate a 500 mL expression culture: either LB media for IPTG induced expression or autoinduction superbroth media (Formedium, #AIMSB0210). In LB media, expression was induced at an OD of 0.7-1.0 by the addition of IPTG to a final concentration of 0.5 mM. The culture was then grown at 18° C. overnight. In autoinduction expression, the temperature was dropped to 18° C. once an OD of 0.7-1.0 was observed then grown overnight. The cells were harvested by centrifugation and frozen until use.

Thawed cells were resuspended into resuspension buffer: 20 mM Tris-HCl pH 8.0, 150 mM NaCl, and DNase I (Merck #4716728001) and lysed by sonication. The lysate was clarified by centrifugation at 23,000 rcf for 15 mins at 4° C. The supernatant was loaded onto 5 mL of NiNTA resin (Cytiva, #17-5248-02) at a flow-rate of 0.5 mL/min. The resin was washed with the same buffer as above containing 20 mM imidazole. Mpro protein was eluted using the same buffer containing 250 mM imidazole. The target protein was further purified using a Superdex S75 16/60 μg (GE, #GE28-9893-33) column in resuspension buffer. Protein purity was assessed by SDS-PAGE and identity confirmed by mass spectrometry. Purified protein was concentrated and frozen until later use.

### SARS-CoV-2 Mpro Enzyme Assay

The activity of SARS-Cov-2 Mpro was determined in a Fluorescence Resonance Energy Transfer (FRET)-based enzymatic assay using FRET Substrate Dabcyl-KTSAVLQSGFRKM-E(Edans)-Amide. In brief, 100 nL of test compounds (concentrations ranging from 10 μM to 0.00051 μM) was preincubated with 5 μL of 5 nM (final concentration) Mpro enzyme for 20 min at room temperature in an assay buffer containing 20 mM Tris (pH 7.5), 100 mM NaCl and 1 mM EDTA. Reaction was initiated by addition of 5 μL of 25 μM (final concentration) of FRET substrate (Dabcyl-KTSAVLQSGFRKM-E (Edans)-Amide). The resulting fluorescent intensity at Ex=360 nm/Em=490 nm was measured every 90 s over the course of 60 min at room temperature using a PHERAstar plate reader (BMG Labtech). Using MARS software (BMG Labtech), the linear portion of the reaction was selected and the rate in RFU per minute calculated. Boceprevir was used a reference standard compound. pIC₅₀ and pKi were determined using 4PL GraphPad Prism and data were represented as a mean n=2±SD. pKi values of compounds of the invention are shown in Table 2.

TABLE 2

| | SARS-CoV-2 Mpro |
|---|---|
| Example No. | enzyme pKi assay pKi |
| 1 | 7.8 |
| 2 | 7.3 |
| 3 | 7.1 |
| 4 | 7.3 |
| 5 | 6.8 |
| 6 | 6.7 |
| 7 | 6.7 |
| 8 | 6.5 |
| 9 | 6.5 |
| 10 | 6.5 |
| 11 | 7.2 |
| 12 | 6.5 |
| 13 | 6.5 |
| 14 | 7.8 |
| 15 | 8.4 |
| 16 | 7.3 |
| 17 | 6.9 |
| 18 | 7.3 |
| 19 | 7.3 |
| 20 | 7.2 |
| 21 | 6.8 |
| 22 | 7.8 |
| 23 | <5.3 |
| 24 | Not tested |
| 25 | 6.6 |
| 26 | 7.4 |
| 27 | <5.3 |
| 28 | 8.2 |
| 29 | 6.6 |
| 30 | 6.8 |
| 31 | 7.0 |
| 32 | 7.2 |
| 33 | 6.8 |
| 34 | 8.4 |
| 35 | 7.4 |

1. A compound of Formula (1 b): 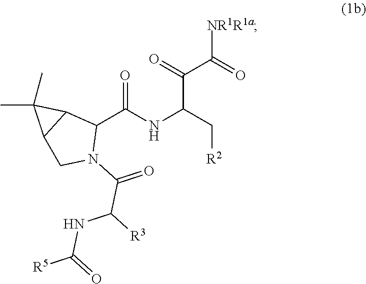
or a salt thereof, wherein:
R¹ and R^{1a} are independently H, a C₁₋₆ saturated hydrocarbon group optionally substituted with 1 to 6 fluorine or chlorine atoms or a benzyl group optionally substituted with 1 to 6 fluorine or chlorine atoms or R¹ and R^{1a} are linked together to form a saturated ring optionally containing an additional heteroatom;
R² is a C₃₋₅ saturated hydrocarbon group containing a cycloalkyl group optionally substituted with one or more substituents chosen from fluorine or hydroxyl;
R³ is a saturated group containing 3-5 carbon atoms and optionally containing a cycloalkyl group or optionally containing a saturated ring containing an oxygen heteroatom and optionally substituted with one or more substituents chosen from fluorine, or hydroxyl or R³ is CH₂aryl, CH(CH₃)aryl or C(CH₃)₂aryl; and
R⁵ is a C₂₋₈ hydrocarbon group, optionally containing one or more rings or a double bond and which is optionally substituted with one or more groups selected from fluorine; chlorine; bromine; cyano; hydroxy; methoxy; amino; or a cycloalkyl, heterocycloalkyl, aryl or heteroaryl group.

2. The compound according to claim 1, wherein R¹ is H, CH₃, benzyl, cyclopropyl or

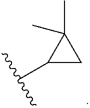 [SMILES: *C1CC1(C)C]

3. The compound according to claim 1, wherein R^{1a} is H.

4. The compound according to claim 1, wherein R¹ and R^{1a} are both H.

5. The compound according to claim 1, wherein R¹ and R^{1a} are both —CH₃.

6. The compound according to claim 1, wherein R¹ and R^{1a} are linked together to form a saturated ring of 3 to 6 atoms.

7. The compound according to claim 1, wherein R¹ and R^{1a} are linked together to form an azetidine or aziridine ring.

8. The compound according to claim 1, wherein R² is selected from the group consisting of:

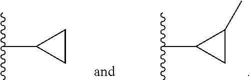

9. The compound according to claim 1, wherein R³ is selected from the group consisting of:

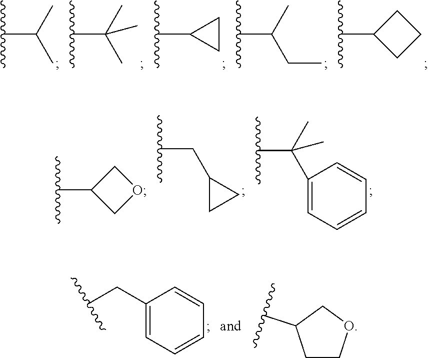

10. The compound according to claim 1,
wherein R⁵ is selected from the group consisting of:

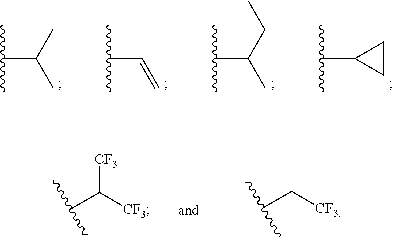

11. The compound according to claim 1, which is a compound of formula (3b): 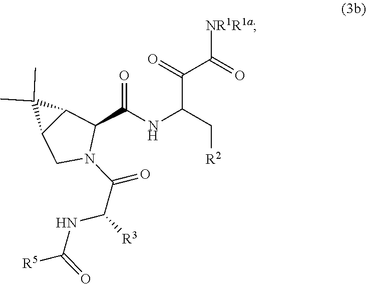
or a salt thereof.

12. The compound according to claim 1, which is selected from the group consisting of:
(1 R,2S,5S)—N-(4-Amino-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-[3-Amino-1-(cyclopropylmethyl)-2,3-dioxo-propyl]-3-[(2S)-2-(cyclopropanecarbonylamino)-3-methylbutanoyl]-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-alloisoleucyl)-6,6-dimethyl-3-azabicyclo[3.1.0]hexane-2-carboxamide;
(1 R,2S,5S)-3-(Acryloyl-L-valyl)-N-(4-amino-1-cyclopropyl-3,4-dioxobutan-2-yl)-6,6-dimethyl-3-azabicyclo[3.1.0]hexane-2-carboxamide;
(1 R,2S,5S)—N-(4-Amino-1-((1 R,2S)-2-methylcyclopropyl)-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3-cyclopropyl-2-isobutyramidopropanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-6,6-dimethyl-3-(((S)-2-methylbutanoyl)-L-valyl)-3-azabicyclo[3.1.0]hexane-2-carboxamide;
(1 R,2S,5S)—N-(4-Amino-1-cyclopropyl-3,4-dioxobutan-2-yl)-3-((S)-2-(cyclopropanecarboxamido)-2-cyclopropylacetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-cyclopropyl-2-isobutyramidoacetyl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-(isobutyryl-L-valyl)-6,6-dimethyl-3-azabicyclo[3.1.0]hexane-2-carboxamide;
(1 R,2S,5S)—N-(1-Cyclopropyl-4-(cyclopropylamino)-3,4-dioxobutan-2-yl)-3-(isobutyryl-L-valyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(2-isobutyramido-2-(oxetan-3-yl)acetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-(cyclopropanecarboxamido)-3,3-dimethylbutanoyl)-6,6-dimethyl-3-azabicyclo[3.1.0]hexane-2-carboxamide;
(1 R,2S,5S)-3-((S)-2-Acrylamido-3,3-dimethylbutanoyl)-N-(4-amino-1-cyclopropyl-3,4-dioxobutan-2-yl)-6,6-dimethyl-3-azabicyclo[3.1.0]hexane-2-carboxamide;
(1 R,2S,5S)-3-((S)-2-Acrylamido-3,3-dimethylbutanoyl)-N-(4-(benzylamino)-1-cyclopropyl-3,4-dioxobutan-2-yl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-(Benzylamino)-1-cyclopropyl-3,4-dioxobutan-2-yl)-3-((S)-2-(cyclopropanecarboxamido)-3,3-dimethylbutanoyl)-6,6-dimethyl-3-azabicyclo[3.1.0]hexane-2-carboxamide;
(1 R,2S,5S)-3-((S)-2-(Cyclopropanecarboxamido)-3,3-dimethylbutanoyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)-3-((S)-2-Acrylamido-3,3-dimethylbutanoyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)—N-(1-Cyclopropyl-4-(cyclopropylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(1-Cyclopropyl-4-(methylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(1-Cyclopropyl-4-(dimethylamino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-(Aziridin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-cyclobutyl-2-isobutyramidoacetyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-2-isobutyramido-3-methyl-3-phenylbutanoyl)-6,6-dimethyl-3-azabicyclo[3.1.0]hexane-2-carboxamide;
(1 R,2S,5S)—N-(4-(Azetidin-1-yl)-1-cyclopropyl-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)-3-((Cyclopropanecarbonyl)-L-valyl)-N-(1-cyclopropyl-4-(cyclopropylamino)-3,4-dioxobutan-2-yl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(2-isobutyramido-2-(tetrahydrofuran-3-yl)acetyl)-6,6-dimethyl-3-azabicyclo[3.1.0]hexane-2-carboxamide;
(1 R,2S,5S)—N-(1-Cyclopropyl-4-(((S)-2,2-dimethylcyclopropyl)amino)-3,4-dioxobutan-2-yl)-3-((S)-2-isobutyramido-3,3-dimethylbutanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-(isobutyryl-L-phenylalanyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3,3-dimethyl-2-(3,3,3-trifluoro-2-(trifluoromethyl)propanamido)butanoyl)-6,6-dimethyl-3-azabicyclo[3.1.0]hexane-2-carboxamide;
(1 R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide; and
(1 R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide;
or a salt thereof.

13. The compound according to claim 1, wherein said compound has SARS-CoV-2 Mpro inhibitor activity.

14. A pharmaceutical composition comprising a compound as defined in claim 1 and a pharmaceutically acceptable excipient.

15. (canceled)

16. A method of treating SARS-CoV-2 in a subject in need thereof comprising administering an effective therapeutic amount of a compound according to claim 1.

17. A method of treating SARS-CoV-2 in a subject in need thereof comprising administering an effective therapeutic amount of a pharmaceutical composition according to claim 14.

18. A method of treating a disorder associated with SARS-CoV-2 in a subject in need thereof comprising administering an effective therapeutic amount of a compound according to claim 1.

19. A method of treating a disorder associated with SARS-CoV-2 in a subject in need thereof comprising administering an effective therapeutic amount of a pharmaceutical composition according to claim 14.
